SEQUENCE LISTING (4 sequences)

SEQ ID NO: 1 - protein, 39 aa, Homo sapiens
DGDQCETSPCQNQGKCKDGLGEYTCTCLEGFEGKNCELF

SEQ ID NO: 2 - protein, 31 aa, Homo sapiens
  misc_feature (3)..(5): Xaa can be any naturally occurring amino acid
  misc_feature (7)..(8): Xaa can be any naturally occurring amino acid
  misc_feature (10)..(10): Xaa can be any naturally occurring amino acid
  misc_feature (14)..(14): Xaa can be any naturally occurring amino acid
  misc_feature (17)..(18): Xaa can be any naturally occurring amino acid
  misc_feature (25)..(26): Xaa can be any naturally occurring amino acid
  misc_feature (29)..(29): Xaa can be any naturally occurring amino acid
CDXXXCXXKXGNGXCDXXCNNAACXXDGXDC

SEQ ID NO: 3 - DNA, 2324 bp, Homo sapiens
cggaccgtgcaatggcccagcgtaagaatgccaagagcagcggcaacagcagcagcagcggctccggcagcggtagcacgagtgcgggcagcagcagccccggggcccggagagagacaaagcatggaggacacaagaatgggaggaaaggcggactctcgggaacttcattcttcacgtggtttatggtgattgcattgctgggcgtctggacatctgtagctgtcgtttggtttgatcttgttgactatgaggaagttctaggaaaactaggaatctatgatgctgatggtgatggagattttgatgtggatgatgccaaagttttattaggacttaaagagagatctacttcagagccagcagtcccgccagaagaggctgagccacacactgagcccgaggagcaggttcctgtggaggcagaaccccagaatatcgaagatgaagcaaaagaacaaattcagtcccttctccatgaaatggtacacgcagaacatgttgagggagaagacttgcaacaagaagatggacccacaggagaaccacaacaagaggatgatgagtttcttatggcgactgatgtagatgatagatttgagaccctggaacctgaagtatctcatgaagaaaccgagcatagttaccacgtggaagagacagtttcacaagactgtaatcaggatatggaagagatgatgtctgagcaggaaaatccagattccagtgaaccagtagtagaagatgaaagattgcaccatgatacagatgatgtaacataccaagtctatgaggaacaagcagtatatgaacctctagaaaatgaagggatagaaatcacagaagtaactgctccccctgaggataatcctgtagaagattcacaggtaattgtagaagaagtaagcatttttcctgtggaagaacagcaggaagtaccaccagaaacaaatagaaaaacagatgatccagaacaaaaagcaaaagttaagaaaaagaagcctaaacttttaaataaatttgataagactattaaagctgaacttgatgctgcagaaaaactccgtaaaaggggaaaaattgaggaagcagtgaatgcatttaaagaactagtacgcaaataccctcagagtccacgagcaagatatgggaaggcgcagtgtgaggatgatttggctgagaagaggagaagtaatgaggtgctacgtggagccatcgagacctaccaagaggtggccagcctacctgatgtccctgcagacctgctgaagctgagtttgaagcgtcgctcagacaggcaacaatttctaggtcatatgagaggttccctgcttaccctgcagagattagttcaactatttcccaatgatacttccttaaaaaatgaccttggcgtgggatacctcttgataggagataatgacaatgcaaagaaagtttatgaagaggtgctgagtgtgacacctaatgatggctttgctaaagtccattatggcttcatcctgaaggcacagaacaaaattgctgagagcatcccatatttaaaggaaggaatagaatccggagatcctggcactgatgatgggagattttatttccacctgggggatgccatgcagagggttgggaacaaagaggcatataagtggtatgagcttgggcacaagagaggacactttgcatctgtctggcaacgctcactctacaatgtgaatggactgaaagcacagccttggtggaccccaaaagaaacgggctacacagagttagtaaagtctttagaaagaaactggaagttaatccgagatgaaggccttgcagtgatggataaagccaaaggtctcttcctgcctgaggatgaaaacctgagggaaaaaggggactggagccagttcacgctgtggcagcaaggaagaagaaatgaaaatgcctgcaaaggagctcctaaaacctgtaccttactagaaaagttccccgagacaacaggatgcagaagaggacagatcaaatattccatcatgcaccccgggactcacgtgtggccgcacacagggcccacaaactgcaggctccgaatgcacctgggcttggtgattcccaaggaaggctgcaagattcgatgtgccaacgagaccaggacctgggaggaaggcaaggtgctcatctttgatgactcctttgagcacgaggtatggcaggatgcctcatctttccggctgatattcatcgtggatgtgtggcatccggaactgacaccacagcagagacgcagccttccagcaatttagcatgaattcatgcaagcttgggaaactctggagaga

SEQ ID NO: 4 - protein, 758 aa, Homo sapiens [sequence omitted]

(19) United States
(12) Patent Application Publication
(10) Pub. No.: US 2021/0052561 A1
(43) Pub. Date: Feb. 25, 2021

(54) TARGETING ASPARTATE BETA-HYDROXYLASE SUPPRESSES TUMOR MALIGNANCY AND METASTASIS

(71) Applicants: Rhode Island Hospital, Providence, RI (US); Midwestern University, Downers Grove, IL (US)

(72) Inventors: Jack R. Wands, Pawtucket, RI (US); Rolf I. Carlson, Providence, RI (US); Mark Jon Olsen, Boston, MA (US)

(21) Appl. No.: 17/001,494

(22) Filed: Aug. 24, 2020

Related U.S. Application Data
(60) Provisional application No. 62/891,423, filed on Aug. 25, 2019.

(51) Int. Cl. A61K 31/443; A61K 31/341; A61K 31/381; A61P 35/00

(52) U.S. Cl. CPC A61K 31/443; A61P 35/00; A61K 31/381; A61K 31/341

(57) ABSTRACT

Disclosed are methods of treating a cell proliferative disease (e.g., cancer) by administering compounds which inhibit the activity of beta-hydroxylase (e.g., ASPH). Further disclosed are methods suppressing metastasis in a cancer cell by contacting the cancer cell with the compounds.

### CROSS-REFERENCES TO RELATED APPLICATIONS

This application claims the benefit of U.S. Provisional Application No. 62/891,423, filed Aug. 25, 2019 which are incorporated herein by reference in entirety and for all purposes.

### INCORPORATION-BY-REFERENCE OF A SEQUENCE LISTING

The sequence listing contained in the file “021486-645001US_Sequence_List.txt”, created on 2020-08-22, file size 11,405 bytes, is incorporated by reference in its entirety herein.

### FIELD OF THE INVENTION

The present invention relates to treating cell proliferation disorders, such as cancer.

### BACKGROUND OF THE INVENTION

Cholangiocarcinoma (CCA) is a highly lethal disease with an extremely low 5-year survival rate of 2%. Recent whole genomic sequencing techniques have identified several new mutations such as IDH1 mutation and fibroblast growth factor receptor 2 (FGFR2) fusion protein in 20% and 16% of CCA, respectively. These findings led to the development of inhibitors for these mutations and those inhibitors have been proposed for clinical trials in CCA. Despite the potential of these inhibitors, there is still approximately 80% of CCA patients without an effective therapy.

The high metastatic potential of CCA usually leads to a grim prognosis. The presence of metastasis is one of the critical determinants for choosing the appropriate therapeutic approach for CCA patients. Although surgical resection is considered an effective treatment, patients with metastasis are normally not candidates for it. Thus, chemotherapies, such as gemcitabine and cisplatin, are generally considered for CCA patients with unresectable tumors. But systemic chemotherapies in CCA patients still has a very poor prognosis. Understanding the molecular pathogenesis of CCA metastasis may restrict CCA tumor mobility and render the possibility of tumor resection in certain CCA patients.

### SUMMARY OF THE INVENTION

Provided are, inter alia, aspartate beta-hydroxylase inhibitors that reduce tumor growth or metastasis. Methods of reducing or suppressing tumor growth, malignancy, or metastasis of asparatyl (asparaginyl) beta-hydroxylase (ASPH) overexpressing tumors are also within the invention.

In one aspect, provide is a method of treating a cell proliferative disease comprising administering a therapeutically effective amount of an ASPH inhibitor to a subject in need thereof. Such a subject is identified as comprising a tumor cell that is characterized by at least 10%, 20%, 30% 40% 50%, 75%, 85%, 95%, 2-fold, 5-fold, 10-fold or more of ASPH compared to a normal non-cancer cell of the same or equivalent tissue or organ type.

In some embodiments, the ASPH inhibitor has a structure of

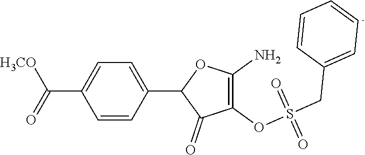 [SMILES: COC(=O)c1ccc(C2OC(N)=C(OS(=O)(=O)Cc3ccccc3)C2=O)cc1]

The cell proliferative disease includes a cancer. In some embodiments, cells of the cancer express greater level of ASPH than normal cells. In some embodiments, the ASPH inhibitor suppresses or reduces metastasis of the cancer cells and/or growth/proliferation of the cancer cells.

The cancer includes liver cancer, pancreatic cancer, lung cancer, colon cancer, breast cancer, prostatic cancer, or brain cancer. In some embodiments, the liver cancer includes hepatocellular cancer, or cholangiocarcinoma. In some embodiments, the lung cancer comprises small cell lung cancer, non-small cell lung cancer, or metastatic lung cancer. In some embodiments, the brain cancer includes gliomas, meningioma, astrocytomas, or glioblastoma. In some embodiments, the breast cancer comprises ductal carcinoma, triple negative breast cancer, inflammatory breast cancer, metastatic breast cancer, medullary carcinoma, tubular carcinoma, or mucinous carcinoma.

In some embodiments, the ASPH inhibitor has a structure of:

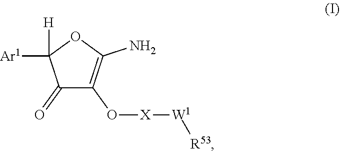

or a pharmaceutically acceptable salt thereof, wherein

Ar¹ is substituted or unsubstituted C₆-C₂₀ aryl or 5 to 20-membered heteroaryl;

X is —C(O)—, —C(S)—, or —S(O)₂—;

W¹ is a single bond, —O—, —CR⁵⁰R⁵¹—, or —NR⁵²— when X is —C(O)—, or W¹ is a single bond, —CR⁵⁰R⁵¹—, or —NR⁵²— when X is —SO₂—; and

each of R⁵⁰, R⁵¹, R⁵², and R⁵³ independently is selected from the group consisting of hydrogen, substituted or unsubstituted C₁-C₆ alkyl, substituted or unsubstituted C₂-C₆ alkenyl, substituted or unsubstituted C₂-C₆ alkynyl, substituted or unsubstituted C₆-C₂₀ aryl, substituted or unsubstituted C₇-C₂₆ arylalkyl, substituted or unsubstituted 5 to 20-membered heteroaryl, and substituted or unsubstituted 6-26 membered heteroarylalkyl.

In some embodiments, R⁵³ is unsubstituted C₁-C₆ alkyl or phenyl, or C₁-C₆ alkyl or phenyl substituted with one or more substituents selected from halogen, —OH, —CN, —COOCH₃, and amino.

In some embodiments, the ASPH inhibitor has a structure of:

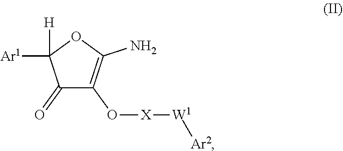

or a pharmaceutically acceptable salt thereof,

wherein

each of Ar¹ and Ar² independently is unsubstituted C₆-C₁₄ aryl, unsubstituted 5 to 14-membered heteroaryl, or C₆-C₁₄ aryl or 5 to 14-membered heteroaryl each substituted with one or more substituents selected from the group consisting of halogen, —CN, —NO₂, —NO, —N₃, —ORₐ, —NRₐR22_{b}, —C(O)Rₐ, —C(O)ORₐ, —C(O)NRₐR_{b}, —NR_{b}C(O)Rₐ, —S(O)_{b}Rₐ, —S(O)_{b}NRₐR_{b}, or —R_{S1}, in which R_{S1} is C₁-C₆ alkyl, C₂-C₆ alkenyl, C₂-C₆ alkynyl, C₃-C₈ cycloalkyl, C₆-C₁₀ aryl, 5- or 6-membered heteroaryl, or 4 to 12-membered heterocycloalkyl, b is 0, 1, or 2, each of Rₐ and R_{b}, independently is H or R_{S2}, and R_{S2} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; and each of R_{S1} and R_{S2}, is optionally substituted with one or more substituents selected from the group consisting of halo, OH, oxo, C(O)OH, C(O)O—C₁-C₆ alkyl, CN, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

In some embodiments, X is C(O) and W¹ is —O—.

In some embodiments, X is —S(O)₂— and W¹ is —CR⁵⁰R⁵¹— or a single bond.

In some embodiments, each of R⁵, R⁵¹, and R⁵² independently is H, unsubstituted C₁-C₆ alkyl, or C₁-C₆ alkyl substituted with one or more substituents selected from halo, OH, CN, and amino.

In some embodiments, each of Ar¹ and Ar² independently is selected from phenyl, 1-naphthyl, 2-naphthyl, 2-furanyl, 2-thiazolyl, 2-pyridyl, 3-pyridyl, 4-pyridyl, 2-quinolinyl, 3-quinolinyl, 4-quinolinyl, 2-chlorophenyl, 3-chlorophenyl, 4-chlorophenyl, 2-fluorophenyl, 3-fluorophenyl, 4-fluorophenyl, 2-trifluoromethylphenyl, 3-trifluoromethylphenyl, 4-trifluoromethylphenyl, 2-cyanophenyl, 3-cyanophenyl, 4-cyanophenyl, 2-carboxymethylphenyl, 3-carboxymethylphenyl, 4-carboxymethylphenyl, 2-methoxyphenyl, 3-methoxyphenyl, 4-methoxyphenyl, 2,3-dichlorophenyl, 2,4-dichlorophenyl, 2,5-dichlorophenyl, 3,4-dichlorophenyl, 3,5-dichlorophenyl, 2,3-difluorophenyl, 2,4-difluorophenyl, 2,5-difluorophenyl, 3,4-difluorophenyl, 3,5-difluorophenyl, 2,3-dimethoxyphenyl, 2,4-dimethoxyphenyl, 2,5-dimethoxyphenyl, 3,4-dimethoxyphenyl, 3,5-dimethoxyphenyl, 2-chloro-6-fluorophenyl, 3-chloro-4-fluorophenyl, 2-chloro-4-fluorophenyl, 4-chloro-3-fluorophenyl, 3-chloro-2-fluorophenyl, 2-chloro-5-fluorophenyl, 4-chloro-2-fluorophenyl, and 5-chloro-2-fluorophenyl.

In some embodiments, the ASPH inhibitor is selected from:

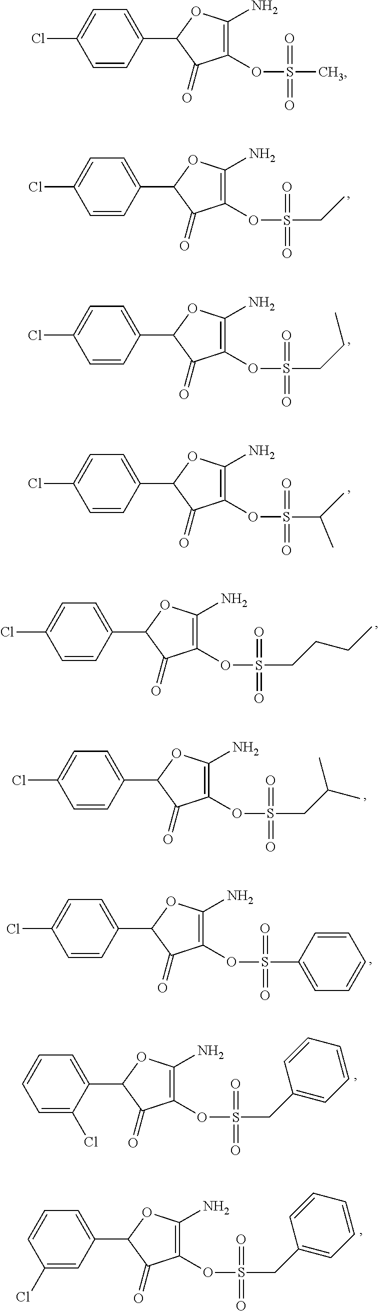

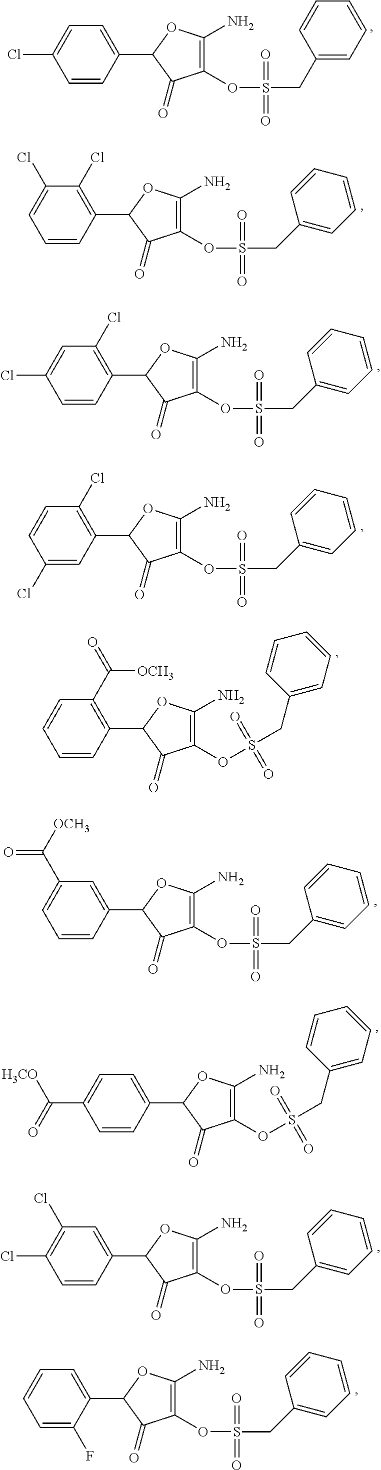

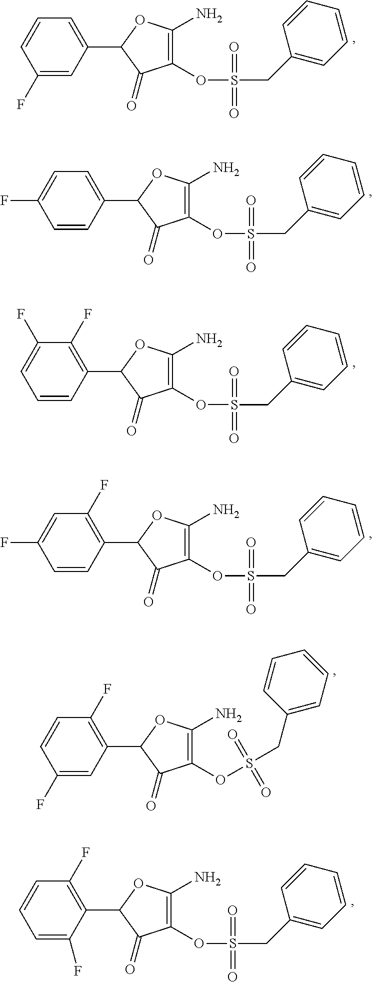 [SMILES: NC1=C(OS(=O)(=O)Cc2ccccc2)C(=O)C(c2cccc(F)c2)O1 ; NC1=C(OS(=O)(=O)Cc2ccccc2)C(=O)C(c2ccc(F)cc2)O1 ; NC1=C(OS(=O)(=O)Cc2ccccc2)C(=O)C(c2cccc(F)c2F)O1 ; NC1=C(OS(=O)(=O)Cc2ccccc2)C(=O)C(c2ccc(F)cc2F)O1 ; NC1=C(OS(=O)(=O)Cc2ccccc2)C(=O)C(c2cc(F)ccc2F)O1 ; NC1=C(OS(=O)(=O)Cc2ccccc2)C(=O)C(c2c(F)cccc2F)O1]

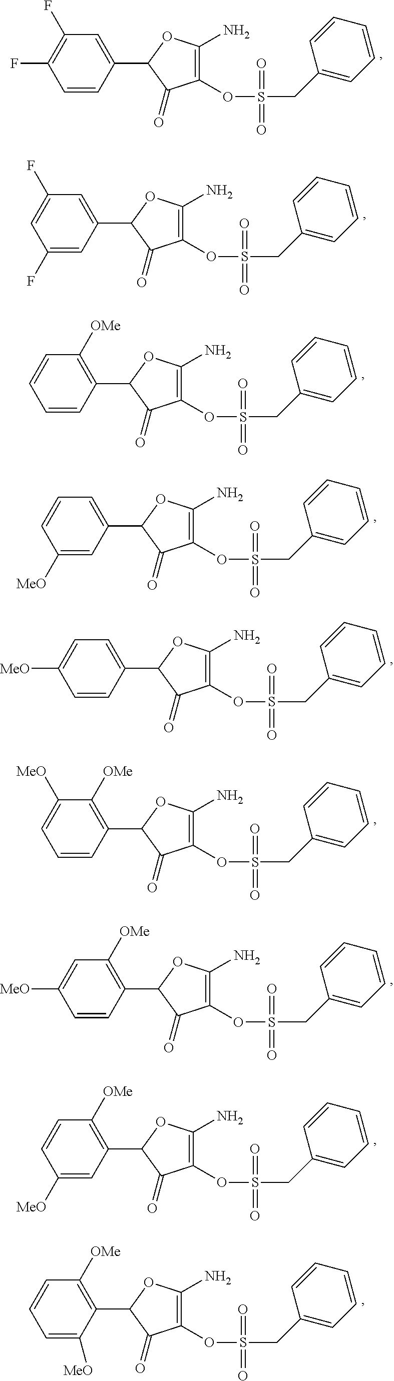

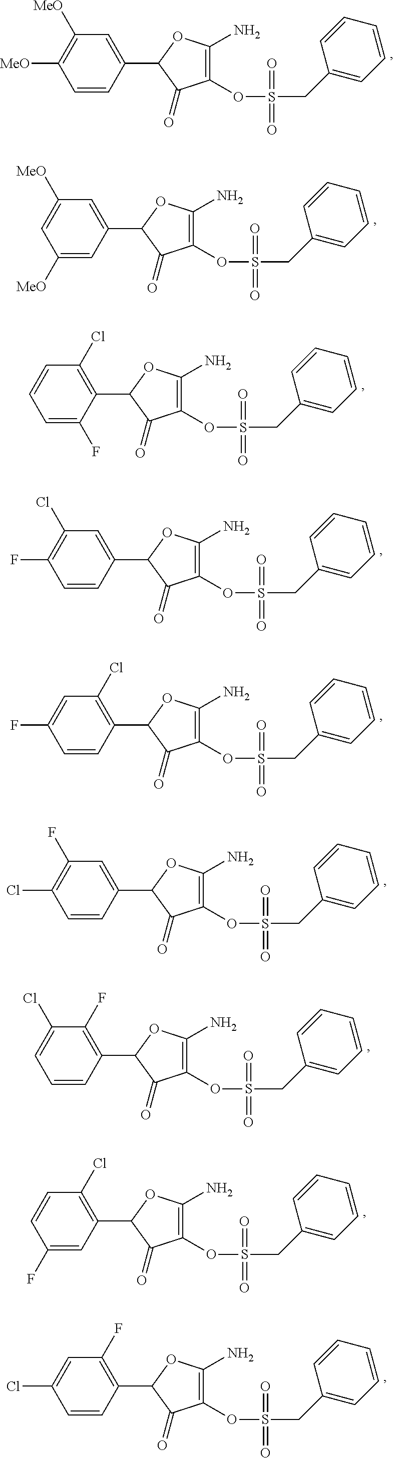

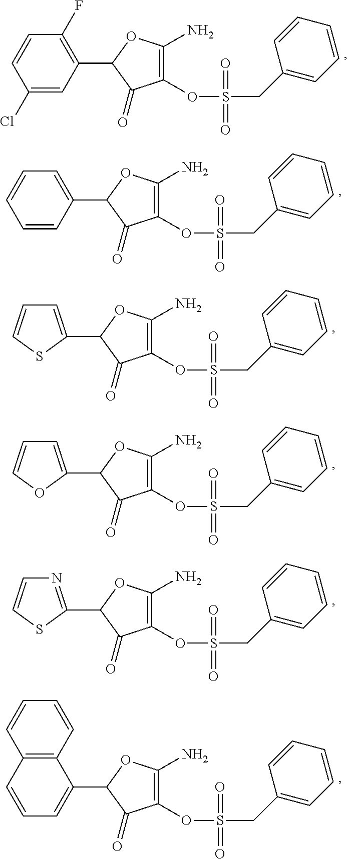 [SMILES: NC1=C(OS(=O)(=O)Cc2ccccc2)C(=O)C(c2cc(Cl)ccc2F)O1 ; NC1=C(OS(=O)(=O)Cc2ccccc2)C(=O)C(c2ccccc2)O1 ; NC1=C(OS(=O)(=O)Cc2ccccc2)C(=O)C(c2cccs2)O1 ; NC1=C(OS(=O)(=O)Cc2ccccc2)C(=O)C(c2ccco2)O1 ; NC1=C(OS(=O)(=O)Cc2ccccc2)C(=O)C(c2nccs2)O1 ; NC1=C(OS(=O)(=O)Cc2ccccc2)C(=O)C(c2cccc3ccccc23)O1]

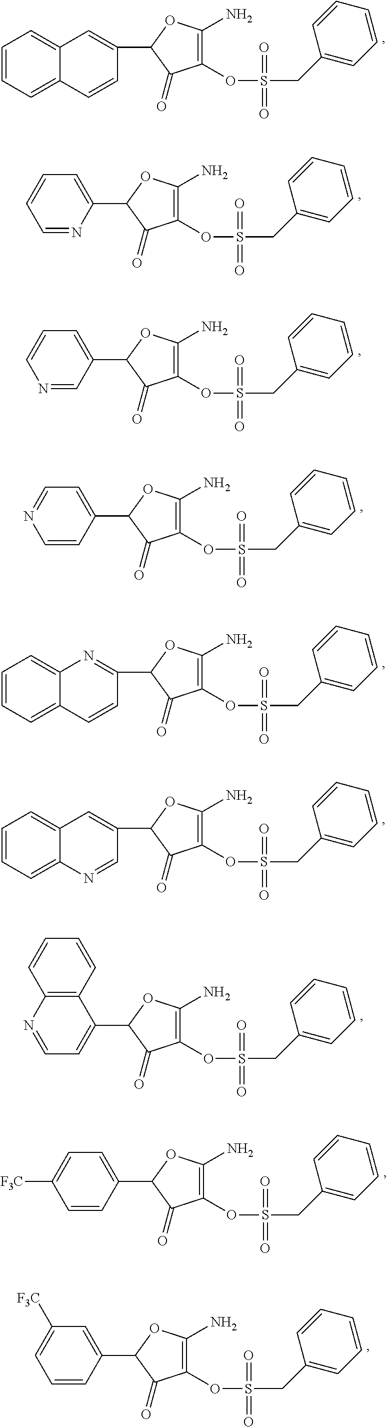

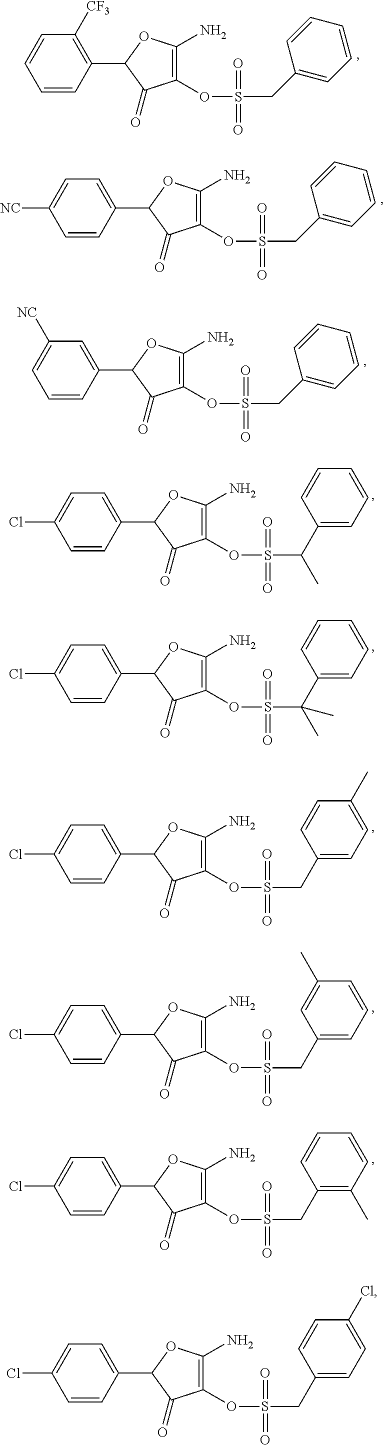

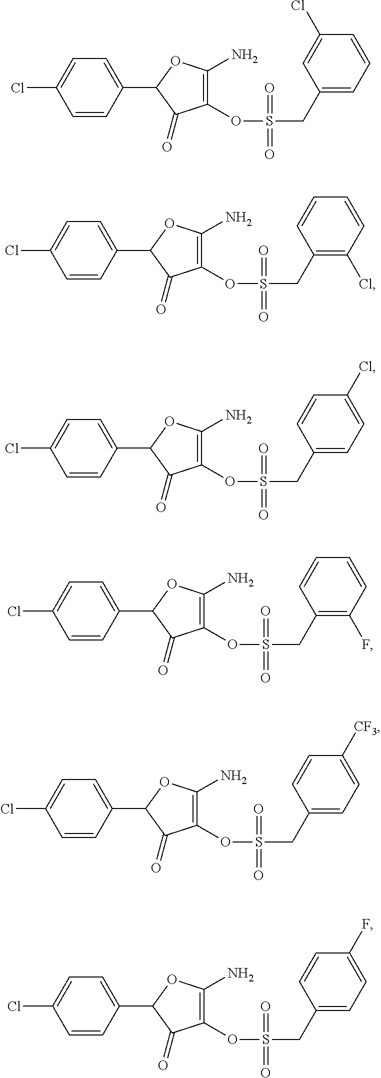 [SMILES: NC1=C(OS(=O)(=O)Cc2cccc(Cl)c2)C(=O)C(c2ccc(Cl)cc2)O1 ; NC1=C(OS(=O)(=O)Cc2ccccc2Cl)C(=O)C(c2ccc(Cl)cc2)O1 ; NC1=C(OS(=O)(=O)Cc2ccc(Cl)cc2)C(=O)C(c2ccc(Cl)cc2)O1 ; NC1=C(OS(=O)(=O)Cc2ccccc2F)C(=O)C(c2ccc(Cl)cc2)O1 ; NC1=C(OS(=O)(=O)Cc2ccc(C(F)(F)F)cc2)C(=O)C(c2ccc(Cl)cc2)O1 ; NC1=C(OS(=O)(=O)Cc2ccc(F)cc2)C(=O)C(c2ccc(Cl)cc2)O1]

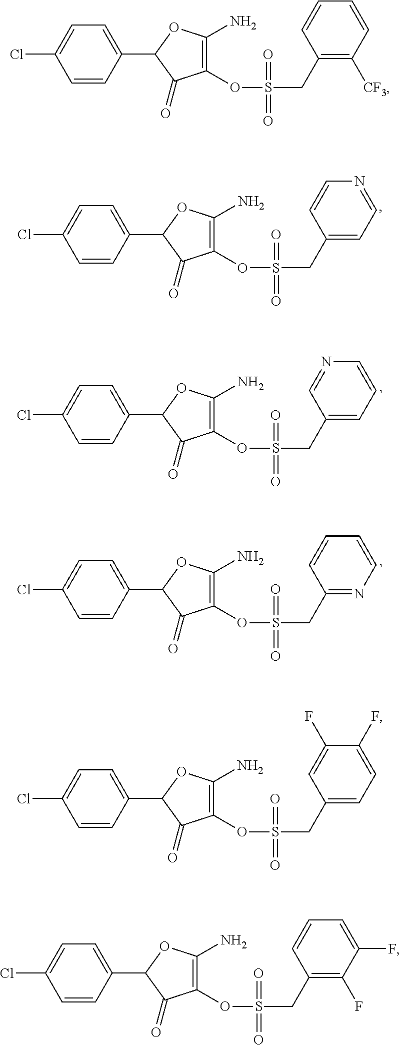 [SMILES: NC1=C(OS(=O)(=O)Cc2ccccc2C(F)(F)F)C(=O)C(c2ccc(Cl)cc2)O1 ; NC1=C(OS(=O)(=O)Cc2ccncc2)C(=O)C(c2ccc(Cl)cc2)O1 ; NC1=C(OS(=O)(=O)Cc2cccnc2)C(=O)C(c2ccc(Cl)cc2)O1 ; NC1=C(OS(=O)(=O)Cc2ccccn2)C(=O)C(c2ccc(Cl)cc2)O1 ; NC1=C(OS(=O)(=O)Cc2ccc(F)c(F)c2)C(=O)C(c2ccc(Cl)cc2)O1 ; NC1=C(OS(=O)(=O)Cc2cccc(F)c2F)C(=O)C(c2ccc(Cl)cc2)O1]

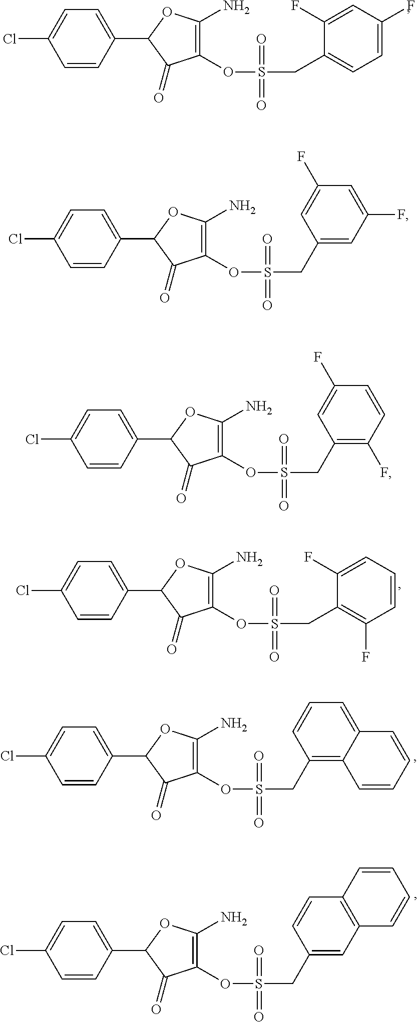 [SMILES: NC1=C(OS(=O)(=O)Cc2ccc(F)cc2F)C(=O)C(c2ccc(Cl)cc2)O1 ; NC1=C(OS(=O)(=O)Cc2cc(F)cc(F)c2)C(=O)C(c2ccc(Cl)cc2)O1 ; NC1=C(OS(=O)(=O)Cc2cc(F)ccc2F)C(=O)C(c2ccc(Cl)cc2)O1 ; NC1=C(OS(=O)(=O)Cc2c(F)cccc2F)C(=O)C(c2ccc(Cl)cc2)O1 ; NC1=C(OS(=O)(=O)Cc2cccc3ccccc23)C(=O)C(c2ccc(Cl)cc2)O1 ; NC1=C(OS(=O)(=O)Cc2ccc3ccccc3c2)C(=O)C(c2ccc(Cl)cc2)O1]

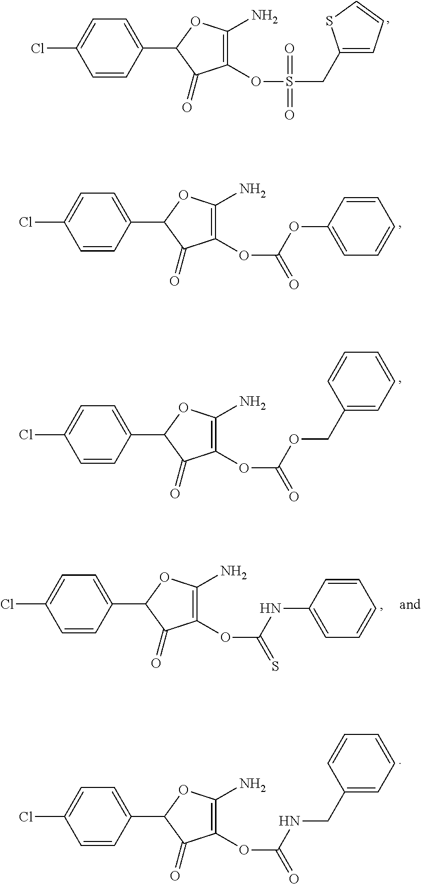 [SMILES: NC1=C(OS(=O)(=O)Cc2cccs2)C(=O)C(c2ccc(Cl)cc2)O1 ; NC1=C(OC(=O)Oc2ccccc2)C(=O)C(c2ccc(Cl)cc2)O1 ; NC1=C(OC(=O)OCc2ccccc2)C(=O)C(c2ccc(Cl)cc2)O1 ; NC1=C(OC(=S)Nc2ccccc2)C(=O)C(c2ccc(Cl)cc2)O1 ; NC1=C(OC(=O)NCc2ccccc2)C(=O)C(c2ccc(Cl)cc2)O1]

The ASPH inhibitor is administered intravenously, orally, subcutaneously, intranasally, intraspinally, intrathecally, intramuscularly, intrabronchially, intrarectally, intraocularly, intravaginally, or by surgical implantation.

In an aspect, provided is a method of suppressing metastasis in a cancer cell contacting the cancer cell with a therapeutically effective amount of an asparatyl (asparaginyl) beta-hydroxylase (ASPH) inhibitor.

The ASPH inhibitor has a structure of:

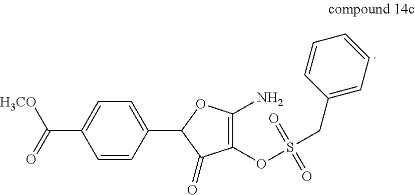 [SMILES: COC(=O)c1ccc(C2OC(N)=C(OS(=O)(=O)Cc3ccccc3)C2=O)cc1]

The cancel cell expresses greater level of ASPH than a normal cell. In some embodiments, the cancer cell is from liver cancer, pancreatic cancer, lung cancer, colon cancer, breast cancer, prostatic cancer, or brain cancer. In some embodiments, the liver cancer includes hepatocellular cancer, or cholangiocarcinoma. In some embodiments, the lung cancer includes small cell lung cancer, non-small cell lung cancer, or metastatic lung cancer. In some embodiments, the brain cancer includes gliomas, meningioma, astrocytomas, or gliblastoma. In some embodiments, the breast cancer includes ductal carcinoma, triple negative breast cancer, inflammatory breast cancer, metastatic breast cancer, medullary carcinoma, tubular carcinoma, or mucinous carcinoma.

In some embodiments, the ASPH inhibitor has a structure of:

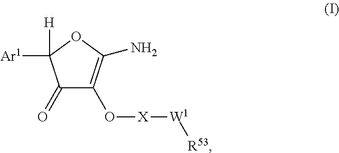

or a pharmaceutically acceptable salt thereof, wherein

Ar¹ is substituted or unsubstituted C₆-C₂₀ aryl or 5 to 20-membered heteroaryl;

X is —C(O)—, —C(S)—, or —S(O)₂—;

W¹ is a single bond, —O—, —CR⁵⁰R⁵¹—, or —NR⁵²— when X is —C(O)—, or W¹ is a single bond, —CR⁵⁰R⁵¹—, or —NR⁵²— when X is —SO₂—; and

each of R⁵⁰, R⁵¹, R⁵², and R⁵³ independently is selected from the group consisting of hydrogen, substituted or unsubstituted C₁-C₆ alkyl, substituted or unsubstituted C₂-C₆ alkenyl, substituted or unsubstituted C₂-C₆ alkynyl, substituted or unsubstituted C₆-C₂₀ aryl, substituted or unsubstituted C₇-C₂₆ arylalkyl, substituted or unsubstituted 5 to 20-membered heteroaryl, and substituted or unsubstituted 6-26 membered heteroarylalkyl.

In some embodiments, R⁵³ is unsubstituted C₁-C₆ alkyl or phenyl, or C₁-C₆ alkyl or phenyl substituted with one or more substituents selected from halogen, —OH, —CN, —COOCH₃, and amino.

In some embodiments, the ASPH inhibitor has a structure of:

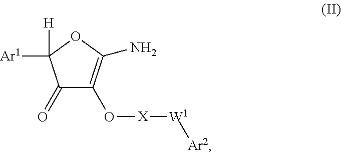

or a pharmaceutically acceptable salt thereof,

wherein

each of Ar¹ and Ar² independently is unsubstituted C₆-C₁₄ aryl, unsubstituted 5 to 14-membered heteroaryl, or C₆-C₁₄ aryl or 5 to 14-membered heteroaryl each substituted with one or more substituents selected from the group consisting of halogen, —CN, —NO₂, —NO, —N₃, —ORₐ, —NRₐR_{b}, —C(O)Rₐ, —C(O)ORₐ, —C(O)NRₐR_{b}, —NR_{b}C(O)Rₐ, —S(O)_{b}Rₐ, —S(O)_{b}NRₐR_{b}, or —R_{S1}, in which R_{S1} is C₁-C₆ alkyl, C₂-C₆ alkenyl, C₂-C₆ alkynyl, C₃-C₈ cycloalkyl, C₆-C₁₀ aryl, 5- or 6-membered heteroaryl, or 4 to 12-membered heterocycloalkyl, b is 0, 1, or 2, each of Rₐ and R_{b}, independently is H or R_{S2}, and R_{S2} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; and each of R_{S1} and R_{S2}, is optionally substituted with one or more substituents selected from the group consisting of halo, OH, oxo, C(O)OH, C(O)O—C₁-C₆ alkyl, CN, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

In some embodiments, X is C(O) and W¹ is —O—.

In some embodiments, X is —S(O)₂— and W¹ is —CR⁵⁰R⁵¹— or a single bond.

In some embodiments, each of R⁵⁰, R⁵¹, and R⁵² independently is H, unsubstituted C₁-C₆ alkyl, or C₁-C₆ alkyl substituted with one or more substituents selected from halo, OH, CN, and amino.

In some embodiments, each of Ar¹ and Ar² independently is selected from phenyl, 1-naphthyl, 2-naphthyl, 2-furanyl, 2-thiazolyl, 2-pyridyl, 3-pyridyl, 4-pyridyl, 2-quinolinyl, 3-quinolinyl, 4-quinolinyl, 2-chlorophenyl, 3-chlorophenyl, 4-chlorophenyl, 2-fluorophenyl, 3-fluorophenyl, 4-fluorophenyl, 2-trifluoromethylphenyl, 3-trifluoromethylphenyl, 4-trifluoromethylphenyl, 2-cyanophenyl, 3-cyanophenyl, 4-cyanophenyl, 2-carboxymethylphenyl, 3-carboxymethylphenyl, 4-carboxymethylphenyl, 2-methoxyphenyl, 3-methoxyphenyl, 4-methoxyphenyl, 2,3-dichlorophenyl, 2,4-dichlorophenyl, 2,5-dichlorophenyl, 3,4-dichlorophenyl, 3,5-dichlorophenyl, 2,3-difluorophenyl, 2,4-difluorophenyl, 2,5-difluorophenyl, 3,4-difluorophenyl, 3,5-difluorophenyl, 2,3-dimethoxyphenyl, 2,4-dimethoxyphenyl, 2,5-dimethoxyphenyl, 3,4-dimethoxyphenyl, 3,5-dimethoxyphenyl, 2-chloro-6-fluorophenyl, 3-chloro-4-fluorophenyl, 2-chloro-4-fluorophenyl, 4-chloro-3-fluorophenyl, 3-chloro-2-fluorophenyl, 2-chloro-5-fluorophenyl, 4-chloro-2-fluorophenyl, and 5-chloro-2-fluorophenyl.

In some embodiments, the ASPH inhibitor is selected from:

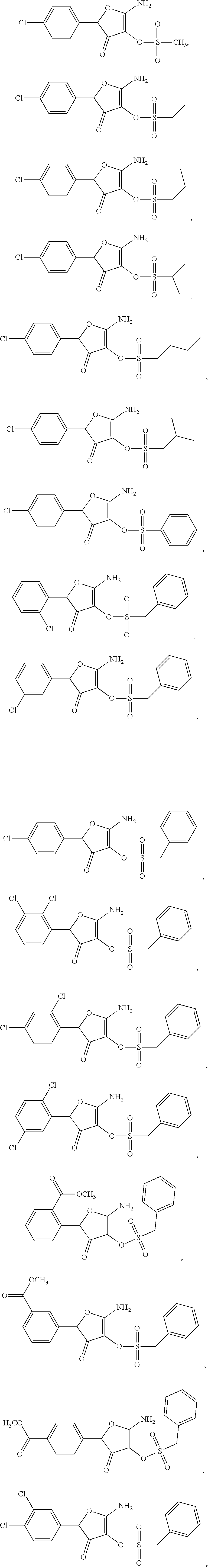

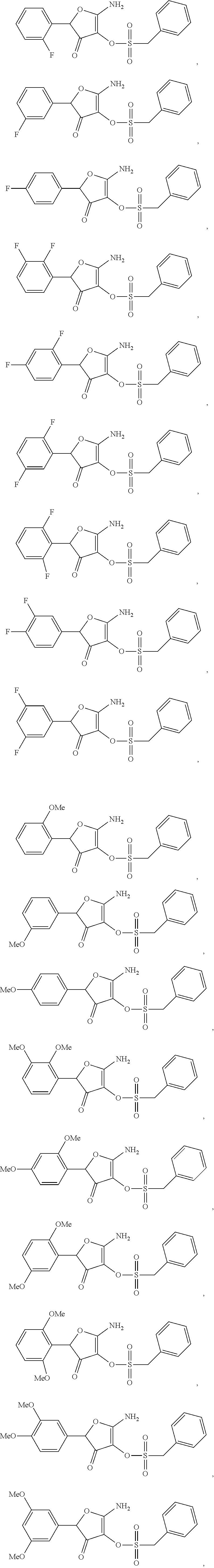

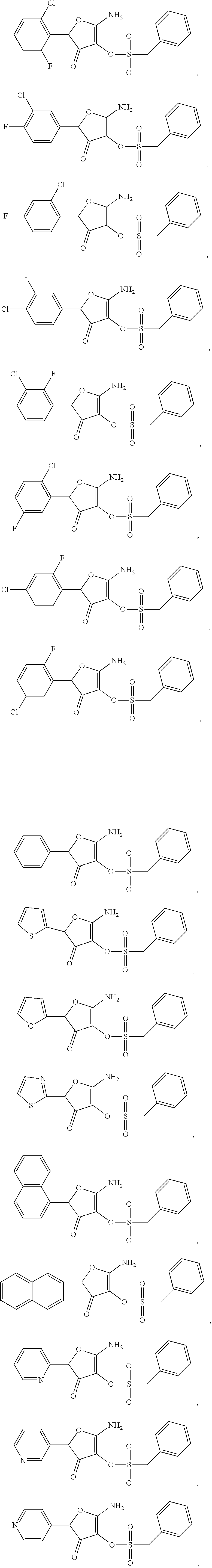

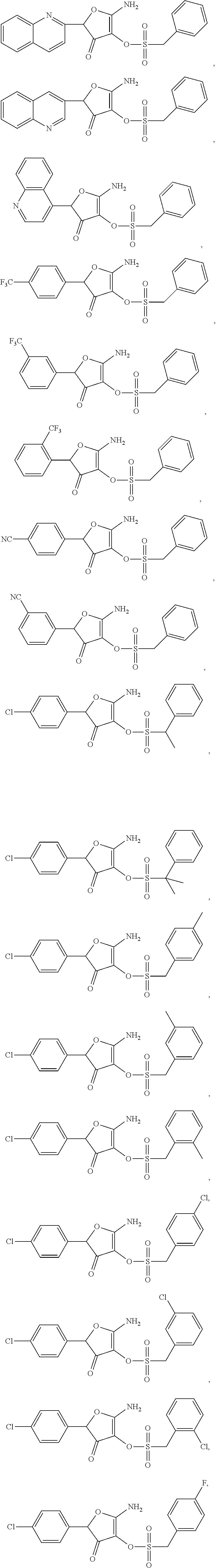

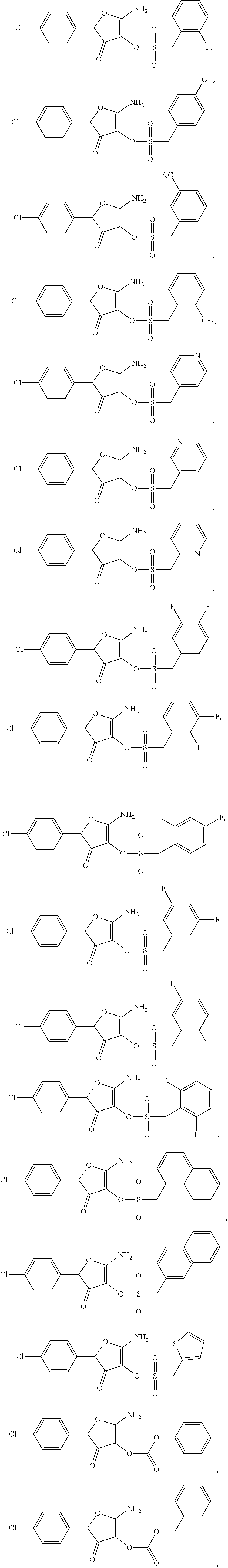

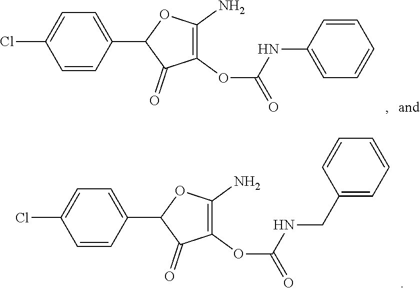 [SMILES: NC1=C(OC(=O)Nc2ccccc2)C(=O)C(c2ccc(Cl)cc2)O1 ; NC1=C(OC(=O)NCc2ccccc2)C(=O)C(c2ccc(Cl)cc2)O1]

The ASPH inhibitor is administered intravenously, orally, subcutaneously, intranasally, intraspinally, intrathecally, intramuscularly, intrabronchially, intrarectally, intraocularly, intravaginally, or by surgical implantation.

Other aspects of the invention are disclosed infra.

### BRIEF DESCRIPTION OF THE DRAWINGS

The patent or application file contains at least one drawing executed in color. Copies of this patent or patent application publication with color drawings will be provided by the Office upon request and payment of the necessary fee.

Other features and advantages of the invention will be apparent from the following description of the preferred embodiments thereof, and from the claims. All references cited herein are incorporated by reference.

FIGS. 1A-1D show that knockdown of ASPH significantly decreases CCA cell migration and invasion.

FIG. 1A shows a representative image of CCA metastatic lung foci. Left panel: macroscopic view. Middle panel: Haemotoxylin and Eosin staining of lung tumor foci. Right panel: Immunohistochemistry staining for ASPH which is highly expressed in the lung foci compared to a normal tissue.

FIG. 1B shows that protein expression levels of ASPH and GAPDH were determined in HuCCT1 and SSP25 cell lines following treatment with shRNA-luciferase (shLuc) or shASPH.

FIG. 1C shows that 24-hour migration assay performed to determine migration in HuCCT1 and SSP25 cell lines treated with shLuc or shASPH. We used 1×10⁵ HuCCT1 and 5×10⁴ SSP25 cells in a migration assay.

FIG. 1D shows that representative 24-hour invasion assay using extracellular matrix (Matrigel®) coated chambers in plated with HuCCT1 and SSP25 CCA cells treated with shLuc or shASPH. We employed 2×10⁵ HuCCT1 and 1×10⁵ SSP25 cells were used. *, p<0.05 versus shLuc.

FIGS. 2A-2B show that targeting ASPH with a specific enzyme inhibitor (compound 14c) substantially suppresses decreases CCA cell migration and invasion.

FIG. 2A shows that a 24-hour transwell migration assay was employed to determine migration of HuCCT1 and RBE CCA cells treated with DMSO or 10 μM compound 14c. We used 5×10⁴ HuCCT1 and 1×10⁵ RBE cells in the migration assay.

FIG. 2B shows that to measure cell invasion assay, 1×10⁵ HuCCT1 and 2.5×10⁴ RBE cells were plated in the upper chamber of a matrix-coated Boyden Chamber transwell system and treated with DMSO (control) or 10 μM compound 14c. * P<0.05 versus DMSO.

FIGS. 3A-3C show that ASPH modulates CCA migration by regulating MMP expression.

FIG. 3A shows that representative example of ASPH and MMP protein levels were determined in HuCCT1 and SSP25 cell lines by treatment with shLuc (control) or shASPH. Right panel: Expression levels of target protein were quantified using ImageJ software. Signal intensities of ASPH and MMP proteins were normalized to those of GAPDH.

FIGS. 3B and 3C show that Cell migration abilities were examined in shLuc or shASPH-treated (FIG. 3B) HuCCT1 (FIG. 3C) and SSP25 in CCA cell lines. *, p<0.05 when compared to DMSO.

FIGS. 4A-4F show that treating primary CCA tumors with the ASPH inhibitor (compound 14c) reduces metastatic spread to the lung.

FIG. 4A shows that cell migration assay was performed with rat BDE-neu CL24 CCA cells treated with DMSO or 10 μM compound 14c.

FIG. 4B shows that the image of the compound 14c containing capsule.

FIG. 4C shows that BDEneu-CL24 cells were inoculated into the liver of young adult Fisher-344 male rats after ligation of the bile duct. Rats received 10 mg/kg (body weight) of the ASPH inhibitor daily for 18 days and sacrificed at day 21 post-surgery

FIG. 4D shows that the images of gross tumor morphology of the lungs prepared in Bouin's solution and derived from control- or compound 14c-treated rats with intrahepatic CCA tumors.

FIG. 4E shows that numbers of metastatic nodules in the lung were determined in the control and compound 14c-treated groups. The statistical analysis was performed using the Mann-Whitney U test. *, p<0.05 versus control.

FIG. 4F shows that representative immunohistochemical images of ASPH, MMP2, and MMP9 expression in control and compound 14c-treated rats.

FIGS. 5A-5B show a scheme for measuring ASPH catalytic activity.

FIG. 5A demonstrates an enzymatic reaction in EGF domain of peptide by converting [14C]α-ketoglutarate into succinate.

FIG. 5B demonstrate an exemplary apparatus including Ca(OH)₂ impregnated glass fiber filger membrane.

FIG. 5C shows exemplary compounds of ASPH inhibitors used in the experiments.

FIGS. 6A-6E show the 3^{rd} generation ASPH small molecule inhibitors and compound 14c that has improved efficacy in suppressing CCA progression.

FIG. 6A shows weight of CCA tumors derived from the rat CCA model produced by BDE-Neu-CL#24 cells and treated with SMI compared to vehicle control (CTRL). The compound 55 (25 mg/kg) was given by i.p injection, and 10 mg/kg of compound 14c via i.p. injection 3 times per week. N=8-10 in each group.

FIG. 6B shows that migration ability of BDE-Neu-CL#24 CCA cells were determined in vehicle control group or compound 55 (2^{nd} generation SMI) treated group.

FIG. 6C shows that migration ability of BDE-Neu-CL#24 CCA cells were determined in vehicle control group or compound 14c treated groups.

FIG. 6D shows that CCA migration was examined in human RBE cells treated with vehicle control and 10 μM compound 55, n=3-6. *p<0.05; **, p<0.01; ***p<0.001.

FIG. 6E shows that CCA migration was examined in human RBE cells treated with vehicle control and 10 μM compound 14c, n=3-6. *p<0.05; **, p<0.01; ***p<0.001.

FIG. 7 shows that body weights of the experimental rats challenged with control (Veh) or compound 14c were measured at the indicated experimental time points, n=8-10. There was no difference between the control and experimental groups.

### DETAILED DESCRIPTION

Aspartate beta-hydroxyase (ASPH) is an alpha-keto-glutarate dependent enzyme highly expressed in CCA but not in healthy bile duct cells. Its function is to hydroxylase epidermal growth factor-like domain containing proteins such as Notch1 and TGFβ1, which have been suggested to participate in CCA malignant progression. In a previous study, ASPH was found to physically interact with Jagged 1 and notch 1. These interactions may promote the Notch1 signaling cascade resulting in malignant progression. It has also been suggested that ASPH expression is correlated with patient prognosis. By targeting ASPH with antisense oligodeoxynucleotide and shRNA, cancer mobility was repressed in vitro. Despite ASPH has been found to be an important factor for malignant progression in vitro, it remains unclear if targeting ASPH with molecular and pharmacological approaches will inhibit CCA metastasis in vivo.

ASPH (a.k.a., AAH) is a member of the α-ketoglutarate-dependent dioxygenase family enzyme. It has a predicted molecular mass of ˜86 kD and catalyzes the hydroxylation of specific Asp (Asparate) and Asn (Asparagine) residues in EGF-like domains of certain receptor proteins such as Notch. Overexpression of ASPH has been observed in a broad range of malignant neoplasms including liver cancer (e.g., hepatocellular carcinoma, and cholangiocellular carcinoma), pancreatic cancer, prostate cancer, breast cancer (e.g., triple negative breast cancer, inflammatory breast cancer, and metastatic breast cancer), gastric cancer (e.g., stomach cancer, gastric lymphoma, gastrointestinal stromal tumor (GIST), and neuroendocrine (carcinoid) tumors), pancreatic cancer, sarcoma (e.g., soft tissue sarcoma and osteosarcoma), brain cancer (e.g., gliomas, meningioma, astrocytomas, and glioblastoma), lung cancer (e.g., small cell lung cancer, non-small cell lung cancer, and metastatic lung cancer), colon cancer, renal cancer, and leukemia (e.g., myeloid leukemia and lymphoid leukemia).

TABLE 1
Percent of human tumors studied that express ASPH by
immunohistochemistry (IHC)

| Tumor tissue type | Number of studied | % positive |
|---|---|---|
| Hepatocellular | 87 | 92 |
| carcinoma | | |
| Cholangiocellular | 27 | 100 |
| carcinoma | | |
| Non-small cell lung | 304 | 82 |
| cancer | | |
| Breast cancer | 47 | 85 |
| Gastric cancer | 51 | 80 |
| Pancreatic cancer | 109 | 97 |
| Soft tissue sarcoma | 30 | 84 |
| Osteosarcoma | 18 | 80 |
| Colon cancer | 41 | 75 |
| Renal cancer | 49 | 83 |
| Myeloid leukemia | 79 | 88 |
| Prostate cancer | 46 | 96 |
| Glioblastoma | 15 | 98 |
| Lymphoid leukemia | 80 | 49 |
| Normal bone marrow | 130 | 0 |
| (control) | | |

However, ASPH has low or negligible expression in normal adult tissues except for proliferating trophoblastic cells of the placenta. In human HCC cell lines, ASPH promotes the motility and invasiveness of tumor cells through upregulation and activation of the Notch signaling cascade. Indeed, ASPH overexpression is reported to be a poor prognostic factor for patients with HCC and predicts early disease recurrence and reduced survival. Especially in colon cancer, there is a significant association between poor surgical outcome and ASPH expression, which is considered an independent risk factor indicating poor prognosis with this disease. Another tumor with high ASPH expression is pancreatic cancer (PC) which is the fourth leading cause of cancer mortality in the United States with a five-year survival rate of 5-6%. PC is an extremely aggressive tumor refractory to most therapies. There is a need to define the molecular pathogenesis of PC and develop more effective treatment strategies. Signaling pathways mediated by ASPH participate in the growth and metastasis of PC during oncogenesis. This surprising discovery on the role of ASPH overexpression in PC pathogenesis indicates that ASPH is a molecular target for therapy and that inhibition of ASPH in this type of cancer leads to clinical benefit.

### Compounds

In an aspect, provided is a compound that inhibits an asparatyl (asparaginyl) beta-hydroxylase (ASPH) inhibitory. The compound, inter alia, may be used for a method of reducing proliferation, migration, invasion, or metastasis of a tumor cell in the treatment of cell proliferative disorder. For example, the method may include administering the compound in a therapeutically effective amount to a subject in need thereof.

In some embodiments, the ASPH inhibitor has a structure of

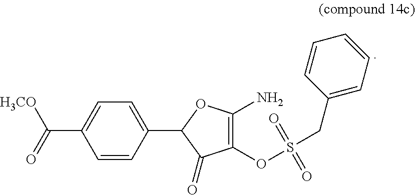 [SMILES: COC(=O)c1ccc(C2OC(N)=C(OS(=O)(=O)Cc3ccccc3)C2=O)cc1]

In some embodiments, the ASPH inhibitory compound is of Formula Ia or Ib:

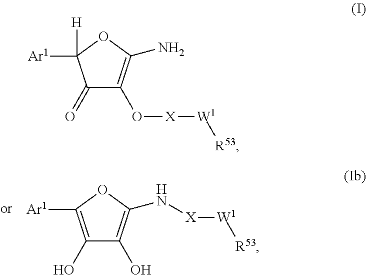

or a salt, ester, metabolite, prodrug, or solvate thereof, wherein
Ar¹ is substituted or unsubstituted C₆-C₂₀ aryl or 5 to 20-membered heteroaryl;

### X is C(O), C(S), or S(O)₂;

W¹ is a single bond, O, CR⁵⁰R⁵¹, or NR⁵² when X is CO and W¹ is a single bond, CR⁵⁰R⁵¹, or NR⁵² when X is SO₂; and
each of R⁵⁰, R⁵¹, R⁵², and R⁵³ independently is selected from the group consisting of hydrogen, substituted or unsubstituted C₁-C₆ alkyl, substituted or unsubstituted C₂-C₆ alkenyl, substituted or unsubstituted C₂-C₆ alkynyl, substituted or unsubstituted C₆-C₂₀ aryl, substituted or unsubstituted C₇-C₂₆ arylalkyl, substituted or unsubstituted 5 to 20-membered heteroaryl, and substituted or unsubstituted 6-26 membered heteroarylalkyl.

In some embodiment, the compound for said use is of Formula I, or a salt, ester, metabolite, prodrug, or solvate thereof. The compound of Formula I may have one or more of the following features when applicable.

In some embodiment, R⁵³ is unsubstituted C₁-C₆ alkyl or phenyl, or C₁-C₆ alkyl or phenyl substituted with one or more substituents selected from halogen, —OH, —CN, —COOCH₃, and amino. In some embodiments, R⁵³ is unsubstituted C₁-C₆ alkyl or phenyl. In some embodiments, R⁵³ is phenyl substituted with one or more substituents selected from halogen, —OH, —CN, —COOCH₃, and amino.

For example, the compound is of Formula II:

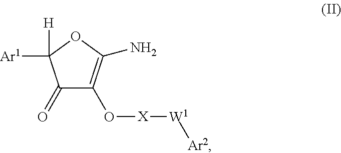

or a salt, ester, metabolite, prodrug, or solvate thereof, wherein:
each of Ar¹ and Ar² independently is unsubstituted C₆-C₁₄ aryl, unsubstituted 5 to 14-membered heteroaryl, or C₆-C₁₄ aryl or 5 to 14-membered heteroaryl each substituted with one or more substituents selected from the group consisting of halo, CN, NO₂, NO, N₃, ORₐ, NRₐR_{b}, C(O)Rₐ, C(O)ORₐ, C(O)NRₐR_{b}, NR_{b}C(O)Rₐ, —S(O)_{b}Rₐ, —S(O)_{b}NRₐR_{b}, or R_{S1}, in which R_{S1} is C₁-C₆ alkyl, C₂-C₆ alkenyl, C₂-C₆ alkynyl, C₃-C₈ cycloalkyl, C₆-C₁₀ aryl, 5- or 6-membered heteroaryl, or 4 to 12-membered heterocycloalkyl, b is 0, 1, or 2, each of Rₐ and R_{b}, independently is H or R_{S2}, and R_{S2} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; and each of R_{S1} and R_{S2}, is optionally substituted with one or more substituents selected from the group consisting of halo, OH, oxo, C(O)OH, C(O)O—C₁-C₆ alkyl, CN, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

In some embodiments, X is C(O) and W¹ is —O—.

In some embodiments, X is —S(O)₂— and W¹ is —CR⁵⁰R⁵¹— or a single bond. In some embodiments, X is —S(O)₂— and W¹ is —CH₂—.

In some embodiments, X is C(O) and W¹ is O, or X is C(S) and W¹ is NR⁵².

In some embodiments, each of R⁵, R⁵¹, and R⁵² independently is H, unsubstituted C₁-C₆ alkyl, or C₁-C₆ alkyl substituted with one or more substituents selected from halo, OH, CN, and amino. In some embodiments, R⁵⁰ is H. In some embodiments, R⁵¹ is H.

For example, each of Ar¹ and Ar² independently is phenyl, naphthyl, or 5 to 10-membered heteroaryl, each of which is optionally substituted with one or more substituents selected from the group consisting of halo, CN, NO₂, NO, N₃, ORₐ, NRₐR_{b}, C(O)Rₐ, C(O)ORₐ, C(O)NRₐR_{b}, NR_{b}C(O)Rₐ, —S(O)_{b}Rₐ, —S(O)_{b}NRₐR_{b}, or R_{S1}, in which R_{S1} is C₁-C₆ alkyl, C₂-C₆ alkenyl, C₂-C₆ alkynyl, C₃-C₈ cycloalkyl, C₆-C₁₀ aryl, 5- or 6-membered heteroaryl, or 4 to 12-membered heterocycloalkyl, b is 0, 1, or 2, each of Rₐ and R_{b}, independently is H or R_{S2}, and R_{S2} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; and each of R_{S1} and R_{S2}, is optionally substituted with one or more substituents selected from the group consisting of halo, OH, oxo, C(O)OH, C(O)O—C₁-C₆ alkyl, CN, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, each of Ar¹ and Ar² independently is phenyl, naphthyl, or 5 to 10-membered heteroaryl, each of which is optionally substituted with one or more substituents selected from the group consisting of halo, CN, NO₂, NO, N₃, ORₐ, NRₐR_{b}, C(O)Rₐ, C(O)ORₐ, or R_{S1}, in which R_{S1} is C₁-C₆ alkyl, each of Rₐ and R_{b}, independently is H or R_{S2}, and R_{S2} is C₁-C₆ alkyl; and each of R_{S1} and R_{S2}, is optionally substituted with one or more substituents selected from the group consisting of halo, OH, C₁-C₆ alkoxyl, amino, mono-C₁-C₆ alkylamino, and di-C₁-C₆ alkylamino. In some embodiments, each of Ar¹ and Ar² is independent phenyl which is optionally substituted with one or more substituents selected from the group consisting of halo, CN, NO₂, NO, N₃, ORₐ, NRₐR_{b}, C(O)Rₐ, C(O)ORₐ, or R_{S1}, in which R_{S1} is C₁-C₆ alkyl, each of Rₐ and R_{b}, independently is H or R_{S2}, and R_{S2} is C₁-C₆ alkyl; and each of R_{S1} and R_{S2}, is optionally substituted with one or more substituents selected from the group consisting of halo, OH, C₁-C₆ alkoxyl, amino, mono-C₁-C₆ alkylamino, and di-C₁-C₆ alkylamino. In some embodiments, Ar¹ is phenyl which is optionally substituted with one or more substituents selected from the group consisting of halo, CN, NO₂, NO, N₃, ORₐ, NRₐR_{b}, C(O)Rₐ, C(O)ORₐ, or R_{S1}, in which R_{S1} is C₁-C₆ alkyl, each of Rₐ and R_{b}, independently is H or R_{S2}, and R_{S2} is C₁-C₆ alkyl; and each of R_{S1} and R_{S2}, is optionally substituted with one or more substituents selected from the group consisting of halo, OH, C₁-C₆ alkoxyl, amino, mono-C₁-C₆ alkylamino, and di-C₁-C₆ alkylamino. In some embodiments, Ar² is phenyl which is optionally substituted with one or more substituents selected from the group consisting of halo, CN, NO₂, NO, N₃, ORₐ, NRₐR_{b}, C(O)Rₐ, C(O)ORₐ, or R_{S1}, in which R_{S1} is C₁-C₆ alkyl, each of Rₐ and R_{b}, independently is H or R_{S2}, and R_{S2} is C₁-C₆ alkyl; and each of R_{S1} and R_{S2}, is optionally substituted with one or more substituents selected from the group consisting of halo, OH, C₁-C₆ alkoxyl, amino, mono-C₁-C₆ alkylamino, and di-C₁-C₆ alkylamino.

For example, each of Ar¹ and Ar² independently is selected from phenyl, 1-naphthyl, 2-naphthyl, 2-furanyl, 2-thiazolyl, 2-pyridyl, 3-pyridyl, 4-pyridyl, 2-quinolinyl, 3-quinolinyl, 4-quinolinyl, 2-chlorophenyl, 3-chlorophenyl, 4-chlorophenyl, 2-fluorophenyl, 3-fluorophenyl, 4-fluorophenyl, 2-trifluoromethylphenyl, 3-trifluoromethylphenyl, 4-trifluoromethylphenyl, 2-cyanophenyl, 3-cyanophenyl, 4-cyanophenyl, 2-carboxymethylphenyl, 3-carboxymethylphenyl, 4-carboxymethylphenyl, 2-methoxyphenyl, 3-methoxyphenyl, 4-methoxyphenyl, 2,3-dichlorophenyl, 2,4-dichlorophenyl, 2,5-dichlorophenyl, 3,4-dichlorophenyl, 3,5-dichlorophenyl, 2,3-difluorophenyl, 2,4-difluorophenyl, 2,5-difluorophenyl, 3,4-difluorophenyl, 3,5-difluorophenyl, 2,3-dimethoxyphenyl, 2,4-dimethoxyphenyl, 2,5-dimethoxyphenyl, 3,4-dimethoxyphenyl, 3,5-dimethoxyphenyl, 2-chloro-6-fluorophenyl, 3-chloro-4-fluorophenyl, 2-chloro-4-fluorophenyl, 4-chloro-3-fluorophenyl, 3-chloro-2-fluorophenyl, 2-chloro-5-fluorophenyl, 4-chloro-2-fluorophenyl, and 5-chloro-2-fluorophenyl. In some embodiments, Ar¹ is 2-carboxymethylphenyl, 3-carboxymethylphenyl, or 4-carboxymethylphenyl. In some embodiments, Ar² is pheny.

In another embodiment, the compound for said use is of Formula Ib, or a salt, ester, metabolite, prodrug, or solvate thereof. The compound of Formula Ib may have one or more of the following features when applicable.

For example, R⁵³ is unsubstituted C₁-C₆ alkyl or C₁-C₆ alkyl substituted with one or more substituents selected from halo, OH, CN, and amino.

For example, R⁵³ is unsubstituted methyl or ethyl.

For example, X is S(O)₂ and W¹ is CR⁵⁰R⁵¹.

For example, X is S(O)₂ and W¹ is a single bond.

For example, X is C(O) and W¹ is O, or X is C(S) and W¹ is NR⁵².

For example, each of R⁵⁰, R⁵¹, and R⁵² independently is H, unsubstituted C₁-C₆ alkyl, or C₁-C₆ alkyl substituted with one or more substituents selected from halo, OH, CN, and amino.

For example, Ar¹ is phenyl, naphthyl, or 5 to 10-membered heteroaryl, each of which is optionally substituted with one or more substituents selected from the group consisting of halo, CN, NO₂, NO, N₃, ORₐ, NRₐR_{b}, C(O)Rₐ, C(O)ORₐ, C(O)NRₐR_{b}, NR_{b}C(O)Rₐ, —S(O)_{b}Rₐ, —S(O)_{b}NRₐR_{b}, or R_{S1}, in which R_{S1} is C₁-C₆ alkyl, C₂-C₆ alkenyl, C₂-C₆ alkynyl, C₃-C₈ cycloalkyl, C₆-C₁₀ aryl, 5- or 6-membered heteroaryl, or 4 to 12-membered heterocycloalkyl, b is 0, 1, or 2, each of Rₐ and R_{b}, independently is H or R_{S2}, and R_{S2} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; and each of R_{S1} and R_{S2}, is optionally substituted with one or more substituents selected from the group consisting of halo, OH, oxo, C(O)OH, C(O)O—C₁-C₆ alkyl, CN, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl. In some embodiments, Ar¹ is phenyl that is substituted with C(O)ORₐ, wherein Rₐ is H, or C₁-C₆ alkyl (e.g., methyl, ethyl, propyl or t-butyl).

For example, Ar¹ is phenyl, naphthyl, or 5 to 10-membered heteroaryl, each of which is optionally substituted with one or more substituents selected from the group consisting of halo, CN, NO₂, NO, N₃, ORₐ, NRₐR_{b}, C(O)Rₐ, C(O)ORₐ, or R_{S1}, in which R_{S1} is C₁-C₆ alkyl, each of Rₐ and R_{b}, independently is H or R_{S2}, and R_{S2} is C₁-C₆ alkyl; and each of R_{S1} and R_{S2}, is optionally substituted with one or more substituents selected from the group consisting of halo, OH, C₁-C₆ alkoxyl, amino, mono-C₁-C₆ alkylamino, and di-C₁-C₆ alkylamino.

For example, Ar¹ is selected from phenyl, 1-naphthyl, 2-naphthyl, 2-furanyl, 2-thiazolyl, 2-pyridyl, 3-pyridyl, 4-pyridyl, 2-quinolinyl, 3-quinolinyl, 4-quinolinyl, 2-chlorophenyl, 3-chlorophenyl, 4-chlorophenyl, 2-fluorophenyl, 3-fluorophenyl, 4-fluorophenyl, 2-trifluoromethylphenyl, 3-trifluoromethylphenyl, 4-trifluoromethylphenyl, 2-cyanophenyl, 3-cyanophenyl, 4-cyanophenyl, 2-carboxymethylphenyl, 3-carboxymethylphenyl, 4-carboxymethylphenyl, 2-methoxyphenyl, 3-methoxyphenyl, 4-methoxyphenyl, 2,3-dichlorophenyl, 2,4-dichlorophenyl, 2,5-dichlorophenyl, 3,4-dichlorophenyl, 3,5-dichlorophenyl, 2,3-difluorophenyl, 2,4-difluorophenyl, 2,5-difluorophenyl, 3,4-difluorophenyl, 3,5-difluorophenyl, 2,3-dimethoxyphenyl, 2,4-dimethoxyphenyl, 2,5-dimethoxyphenyl, 3,4-dimethoxyphenyl, 3,5-dimethoxyphenyl, 2-chloro-6-fluorophenyl, 3-chloro-4-fluorophenyl, 2-chloro-4-fluorophenyl, 4-chloro-3-fluorophenyl, 3-chloro-2-fluorophenyl, 2-chloro-5-fluorophenyl, 4-chloro-2-fluorophenyl, and 5-chloro-2-fluorophenyl. In some embodiments, Ar¹ is 2-carboxymethylphenyl, 3-carboxymethylphenyl, or 4-carboxymethylphenyl. In some embodiments, Ar¹ is 4-carboxymethylphenyl.

Non-limiting examples of ASPH inhibitor include one or more selected from the following compounds:

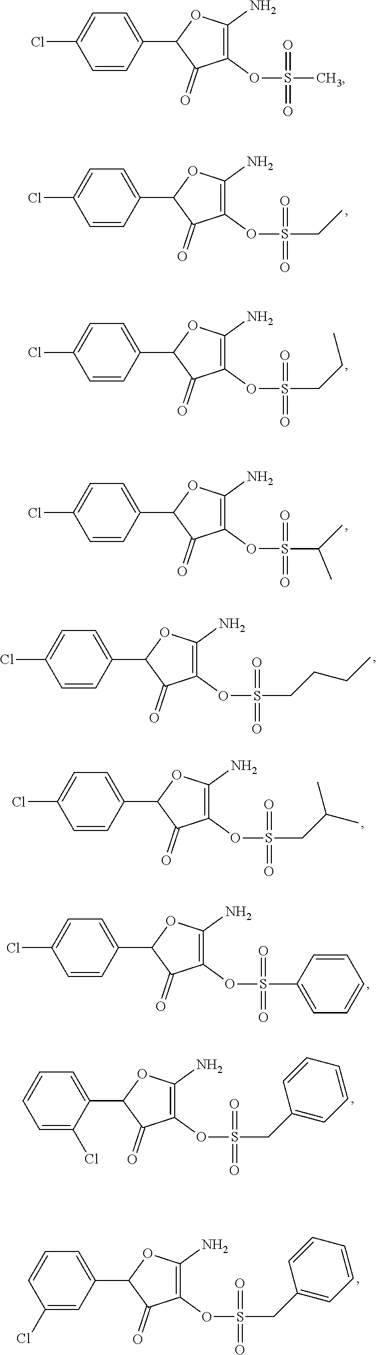

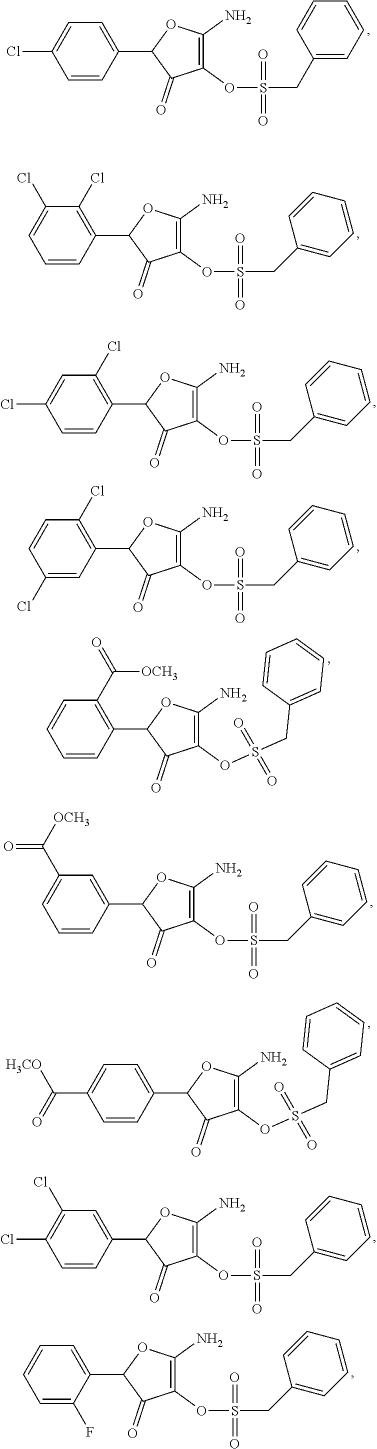

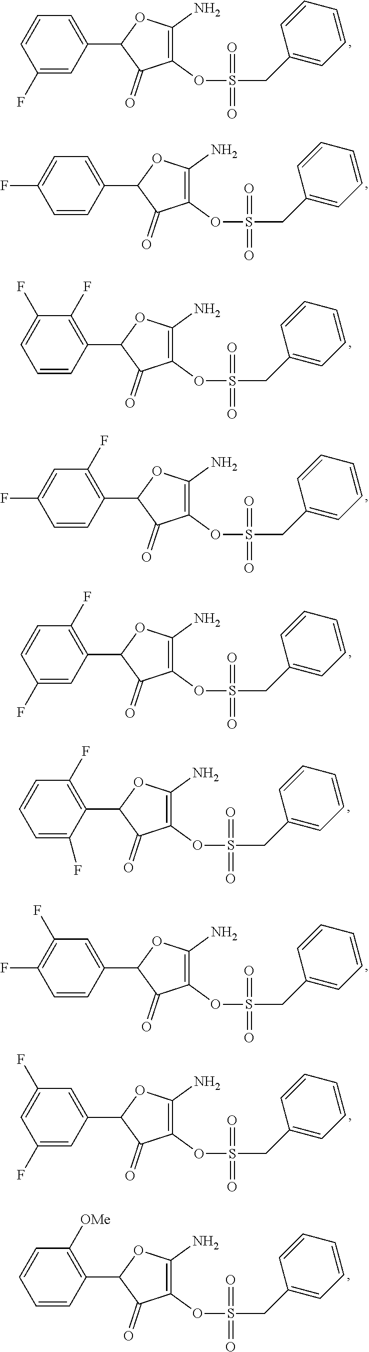

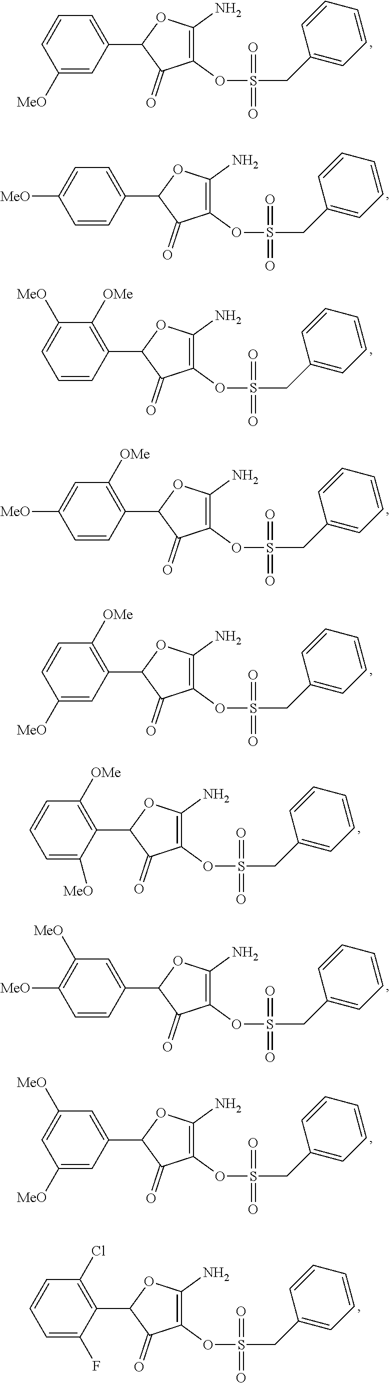

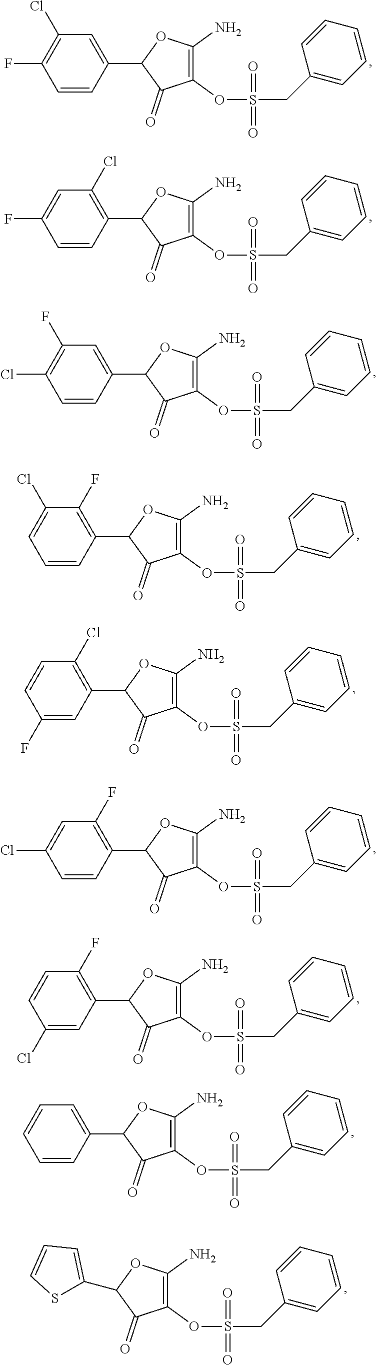

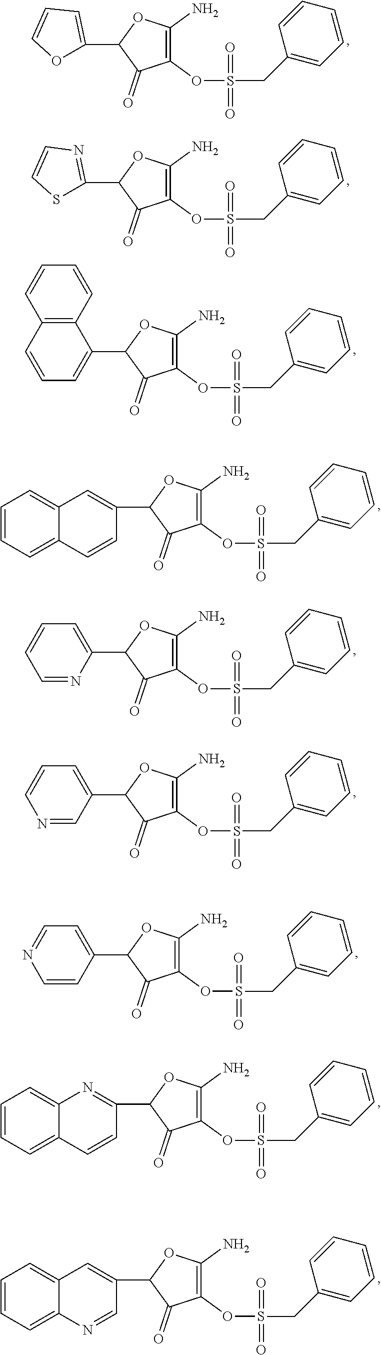

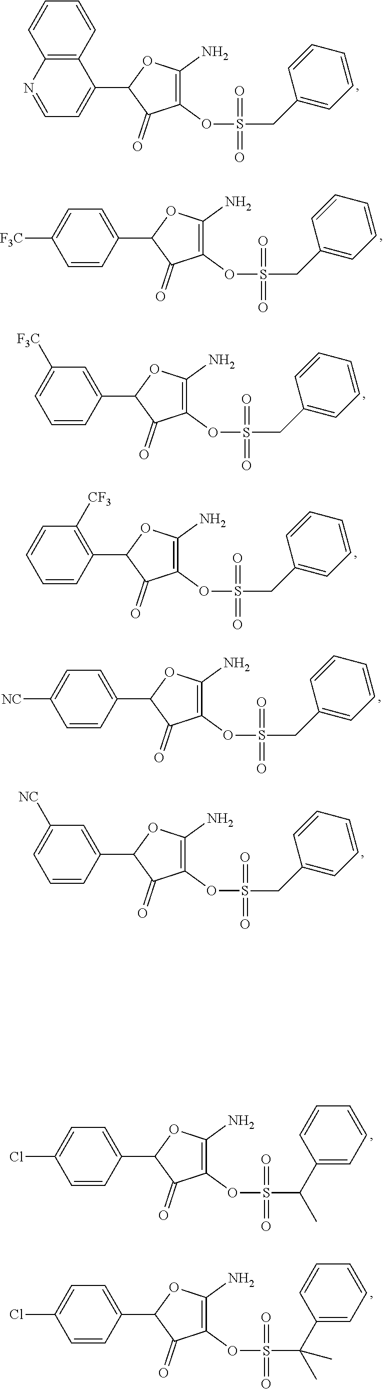

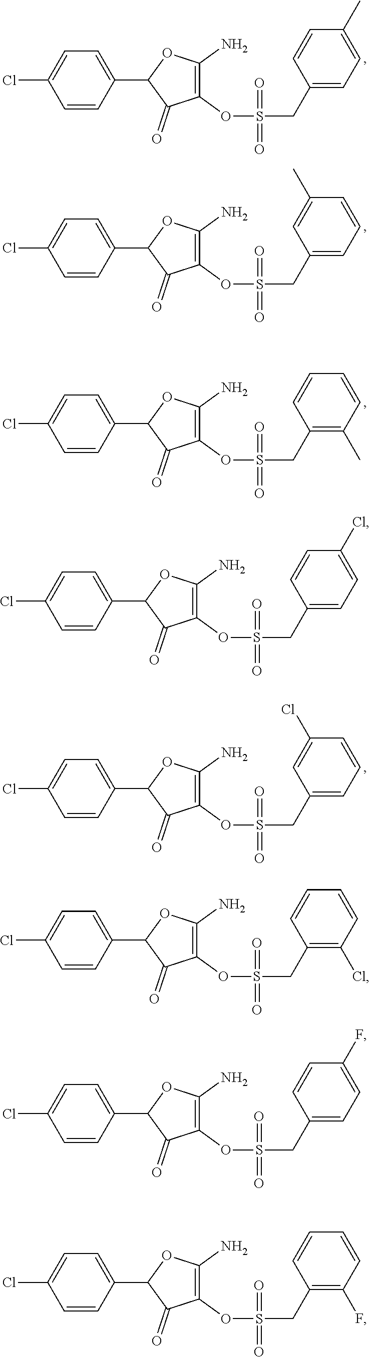

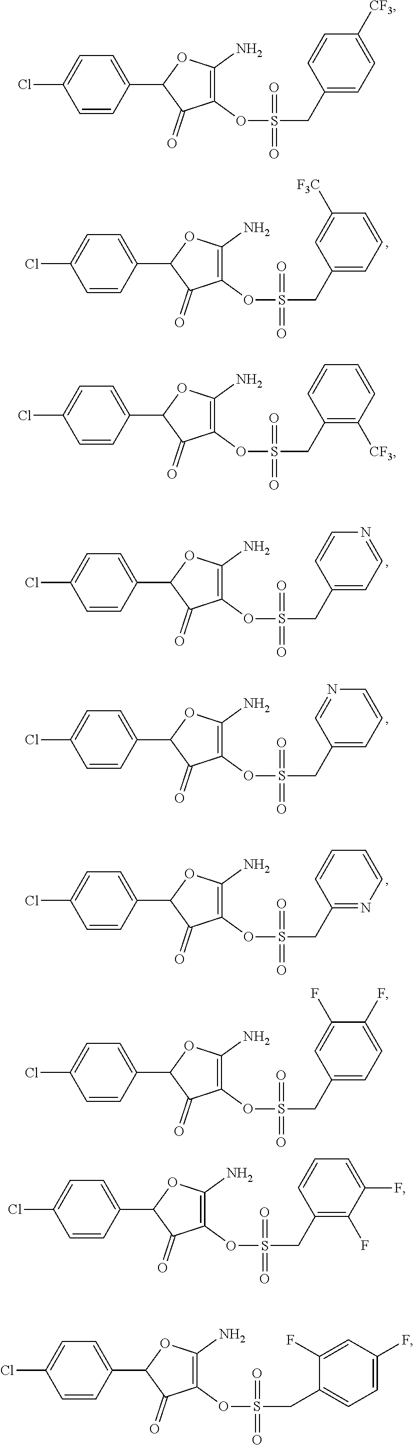

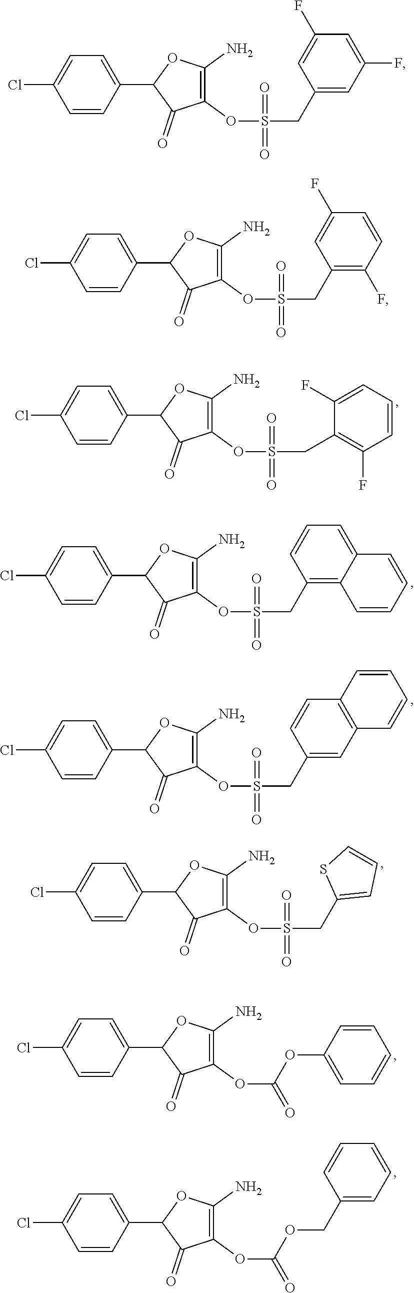

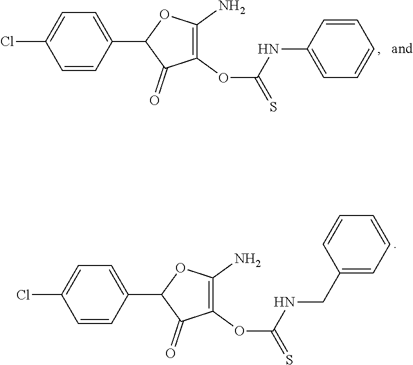 [SMILES: NC1=C(OC(=S)Nc2ccccc2)C(=O)C(c2ccc(Cl)cc2)O1 ; NC1=C(OC(=S)NCc2ccccc2)C(=O)C(c2ccc(Cl)cc2)O1]

In certain embodiments, the ASPH inhibitor includes

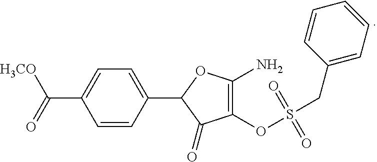 [SMILES: COC(=O)c1ccc(C2OC(N)=C(OS(=O)(=O)Cc3ccccc3)C2=O)cc1]

The ASPH inhibitor is

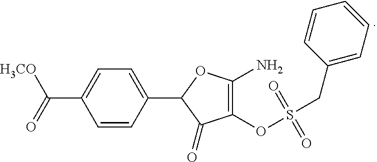 [SMILES: COC(=O)c1ccc(C2OC(N)=C(OS(=O)(=O)Cc3ccccc3)C2=O)cc1]

### Methods

In an aspect, provided is a method of treating a cell proliferative disease comprising administering a therapeutically effective amount of the compound as described herein (e.g., ASPH inhibitor) to a subject in need thereof.

In an aspect, provided is a method of suppressing metastasis in a cancer cell contacting the cancer cell with a therapeutically effective amount of the compound as described herein (e.g., ASPH inhibitor).

In some embodiments, the cell proliferative disease includes a tumor. The cells of the tumor express greater level of ASPH than normal cells. In some embodiments, the cell proliferative disease includes a cancer. The cells of the cancer express greater level of ASPH than normal cells.

In some embodiments, the ASPH inhibitor suppresses metastasis in the cancer cells.

In some embodiments, the cancer includes liver cancer (e.g., hepatocellular carcinoma, and cholangiocellular carcinoma), pancreatic cancer, prostate cancer, breast cancer (e.g., triple negative breast cancer, inflammatory breast cancer, and metastatic breast cancer), gastric cancer (e.g., stomach cancer, gastric lymphoma, gastrointestinal stromal tumor (GIST), and neuroendocrine (carcinoid) tumors), pancreatic cancer, sarcoma (e.g., soft tissue sarcoma and osteosarcoma), brain cancer (e.g., gliomas, meningioma, astrocytomas, and glioblastoma), lung cancer (e.g., small cell lung cancer, non-small cell lung cancer, and metastatic lung cancer), colon cancer, renal cancer, or leukemia (e.g., myeloid leukemia and lymphoid leukemia).

In some embodiments, the liver cancer comprises hepatocellular cancer, or cholangiocarcinoma. In some embodiments, the lung cancer comprises small cell lung cancer, non-small cell lung cancer, or metastatic lung cancer. In some embodiments, the brain cancer comprises gliomas, meningioma, astrocytomas, or glioblastoma. In some embodiments, the breast cancer comprises ductal carcinoma, triple negative breast cancer, inflammatory breast cancer, metastatic breast cancer, medullary carcinoma, tubular carcinoma, or mucinous carcinoma. In some embodiments, the gastric cancer comprises stomach cancer, gastric lymphoma, gastrointestinal stromal tumor (GIST), or neuroendocrine (carcinoid) tumors). In some embodiments, the sarcoma comprises soft tissue sarcoma or osteosarcoma. In some embodiments, the leukemia comprises myeloid leukemia or lymphoid leukemia.

The compound (e.g., the ASPH inhibitor) is administered intravenously, orally, subcutaneously, intranasally, intraspinally, intrathecally, intramuscularly, intrabronchially, intrarectally, intraocularly, intravaginally, or by surgical implantation. In some embodiments, the compound (e.g., the ASPH inhibitor) is administered is administered intravenously, orally, or subcutaneously.

In some embodiments, said compound is administered at a dose of 0.01 to 50 milligrams/kilogram of body weight.

ASPH is a highly conserved cell-surface protein in hepatocellular carcinoma (HCC). Both the liver and the pancreas are derived from an early progenitor cell type and ASPH is expressed in embryo but not in adult tissues. ASPH re-expression was observed in human PC tissue microarrays by immunohistochemical staining (IHS). High level cell surface localization of ASPH was present in 101 of 104 (97%) pancreatic ductal adenocarcinoma with negligible expression in normal pancreas, and other adult human tissues (WO 2014/047519, hereby incorporated by reference). ASPH enhances cell migration, invasion, and metastasis in HCC and also PC. Activation of Notch signaling by ASPH is a final effector mechanism responsible for generation of this highly aggressive and malignant phenotype.

### Biological Properties of ASPH as a Cellular Target

The regulation, expression, and function of ASPH has been observed in many tumors (U.S. Pat. Nos. 6,835,370; 7,094,556; 6,812,206; 6,815,415; 6,797,696; 6,783,758; and U.S. Published Patent Application No. 2005-0123545; hereby incorporated by reference) and ASPH has been found to be overexpressed in pancreatic ductal adenocarcinoma (PC) indicating that it is a therapeutic target for treatment of PC. ASPH catalyzes post-translational hydroxylation of 3-carbons of specific aspartate and asparaginyl residues in epidermal growth factor (EGF)-like domains residing in proteins such as Notch and Jagged (JAG) which are involved in cell growth, differentiation, cellular migration, adhesion, and motility. The catalytic activity resides in the C-terminus and is conferred by the ⁶⁷⁵His residue; mutation to an alanine abolishes ASPH enzymatic and transforming activity. ASPH is overexpressed in tumors derived from the endoderm such as liver, pancreas, colon and lung, and translocates from the endoplasmic reticulum (ER) to the plasma membrane where it becomes accessible to the extracellular environment. It has negligible to very low expression in normal human tissue with the notable exception of the placenta which is an invasive tissue, and its expression there is robust.

Compounds are administered directly into a tumor site or systemically to inhibit ASPH hydroxylase activity.

cDNA sequence of human ASPH
(SEQ ID NO: 3)
cggaccgtgc aatggcccag cgtaagaatg ccaagagcag cggcaacagc agcagcagcg 61
gctccggcag cggtagcacg agtgcgggca gcagcagccc cggggcccgg agagagacaa 121
agcatggagg acacaagaat gggaggaaag gcggactctc gggaacttca ttcttcacgt 181
ggtttatggt gattgcattg ctgggcgtct ggacatctgt agctgtcgtt tggtttgatc 241
ttgttgacta tgaggaagtt ctaggaaaac taggaatcta tgatgctgat ggtgatggag 301
attttgatgt ggatgatgcc aaagttttat taggacttaa agagagatct acttcagagc 361
cagcagtccc gccagaagag gctgagccac acactgagcc cgaggagcag gttcctgtgg 421
aggcagaacc ccagaatatc gaagatgaag caaaagaaca aattcagtcc cttctccatg 481
aaatggtaca cgcagaacat gttgagggag aagacttgca acaagaagat ggacccacag 541
gagaaccaca acaagaggat gatgagtttc ttatggcgac tgatgtagat gatagatttg 601
agaccctgga acctgaagta tctcatgaag aaaccgagca tagttaccac gtggaagaga 661
cagtttcaca agactgtaat caggatatgg aagagatgat gtctgagcag gaaaatccag 721
attccagtga accagtagta gaagatgaaa gattgcacca tgatacagat gatgtaacat 781
accaagtcta tgaggaacaa gcagtatatg aacctctaga aaatgaaggg atagaaatca 841
cagaagtaac tgctccccct gaggataatc ctgtagaaga ttcacaggta attgtagaag 901
aagtaagcat ttttcctgtg gaagaacagc aggaagtacc accagaaaca aatagaaaaa 961
cagatgatcc agaacaaaaa gcaaaagtta agaaaaagaa gcctaaactt ttaaataaat 1021
ttgataagac tattaaagct gaacttgatg ctgcagaaaa actccgtaaa aggggaaaaa 1081
ttgaggaagc agtgaatgca tttaaagaac tagtacgcaa ataccctcag agtccacgag 1141
caagatatgg gaaggcgcag tgtgaggatg atttggctga gaagaggaga agtaatgagg 1201
tgctacgtgg agccatcgag acctaccaag aggtggccag cctacctgat gtccctgcag 1261
acctgctgaa gctgagtttg aagcgtcgct cagacaggca acaatttcta ggtcatatga 1321
gaggttccct gcttaccctg cagagattag ttcaactatt tcccaatgat acttccttaa 1381
aaaatgacct tggcgtggga tacctcttga taggagataa tgacaatgca aagaaagttt 1441
atgaagaggt gctgagtgtg acacctaatg atggctttgc taaagtccat tatggcttca 1501
tcctgaaggc acagaacaaa attgctgaga gcatcccata tttaaaggaa ggaatagaat 1561
ccggagatcc tggcactgat gatgggagat tttatttcca cctgggggat gccatgcaga 1621
gggttgggaa caaagaggca tataagtggt atgagcttgg gcacaagaga ggacactttg 1681
catctgtctg gcaacgctca ctctacaatg tgaatggact gaaagcacag ccttggtgga 1741
ccccaaaaga aacgggctac acagagttag taaagtcttt agaaagaaac tggaagttaa 1801
tccgagatga aggccttgca gtgatggata aagccaaagg tctcttcctg cctgaggatg 1861
aaaacctgag ggaaaaaggg gactggagcc agttcacgct gtggcagcaa ggaagaagaa 1921
atgaaaatgc ctgcaaagga gctcctaaaa cctgtacctt actagaaaag ttccccgaga 1981
caacaggatg cagaagagga cagatcaaat attccatcat gcaccccggg actcacgtgt 2041
ggccgcacac agggcccaca aactgcaggc tccgaatgca cctgggcttg gtgattccca 2101
aggaaggctg caagattcga tgtgccaacg agaccaggac ctgggaggaa ggcaaggtgc 2161
tcatctttga tgactccttt gagcacgagg tatggcagga tgcctcatct ttccggctga 2221
tattcatcgt ggatgtgtgg catccggaac tgacaccaca gcagagacgc agccttccag 2281
caatttagca tgaattcatg caagcttggg aaactctgga gaga
(SEQ ID NO: 3; GenBank Accession No. S83325; codon encoding initiating
methionine is underlined).
Amino acid sequence of human ASPH
(SEQ ID NO: 4)
MAQRKNAKSS GNSSSSGSGS GSTSAGSSSP GARRETKHGG HKNGRKGGLS GTSFFTWFMV 61
IALLGVWTSV AVVWFDLVDY EEVLGKLGIY DADGDGDFDV DDAKVLLGLK ERSTSEPAVP 121
PEEAEPHTEP EEQVPVEAEP QNIEDEAKEQ IQSLLHEMVH AEHVEGEDLQ QEDGPTGEPQ 181
QEDDEFLMAT DVDDRFETLE PEVSHEETEH SYHVEETVSQ DCNQDMEEMM SEQENPDSSE 241
PVVEDERLHH DTDDVTYQVY EEQAVYEPLE NEGIEITEVT APPEDNPVED SQVIVEEVSI 301
FPVEEQQEVP PETNRKTDDP EQKAKVKKKK PKLLNKFDKT IKAELDAAEK LRKRGKIEEA 361
VNAFKELVRK YPQSPRARYG KAQCEDDLAE KRRSNEVLRG AIETYQEVAS LPDVPADLLK 421
LSLKRRSDRQ QFLGHMRGSL LTLQRLVQLF PNDTSLKNDL GVGYLLIGDN DNAKKVYEEV 481
LSVTPNDGFA KVHYGFILKA QNKIAESIPY LKEGIESGDP GTDDGRFYFH LGDAMQRVGN 541
KEAYKWYELG HKRGHFASVW QRSLYNVNGL KAQPWWTPKE TGYTELVKSL ERNWKLIRDE 601
GLAVMDKAKG LFLPEDENLR EKGDWSQFTL WQQGRRNENA CKGAPKTCTL LEKFPETTGC 661
RRGQIKYSIM HPGTHVWPHT GPTNCRLRMH LGLVIPKEGC KIRCANETRT WEEGKVLIFD 721
DSFEHEVWQD ASSFRLIFIV DVWHPELTPQ QRRSLPAI
(SEQ ID NO:4; GenBank Accession No. S83325; His motif is underlined;
conserved sequences within the catalytic domain are designated
by bold type)

(SEQ ID NO:4; GenBank Accession No. S83325; His motif is underlined; conserved sequences within the catalytic domain are designated by bold type)

Methods of inhibiting tumor growth also include administering a compound which inhibits HAAH hydroxylation of a NOTCH polypeptide. For example, the compound inhibits hydroxylation of an EGF-like cysteine-rich repeat sequence in a NOTCH polypeptide, e.g., one containing the consensus sequence

(SEQ ID NO:2)
CDXXXCXXKXGNGXCDXXCNNAACXXDGXDC.

Polypeptides containing an EGF-like cysteine-rich repeat sequence are administered to block hydroxylation of endogenous NOTCH.

ASPH is expressed in many organs during embryogenesis presumably to promote cell motility and migration for cell patterning and organ development; its expression is “shut off” in the adult only to re-emerge during oncogenesis where its function may be required for generation of malignant phenotypes. It appears not to be overexpressed during cell proliferation; however, there is low-level expression in dysplastic ductal cells of pancreatic intraepithelial lesions (PanINs) as well as dysplastic hepatocytes in hepatitis B (HBV) and C (HCV) infected liver. Transcriptional regulation of ASPH is provided by tripartite signaling pathways IN/IGF1/IRS1/MAPK/ERK, IN/IGF1/IRS1/PI3K/AKT, and WNT/β-Catenin. Post-transcriptional regulation of ASPH is mediated by phosphorylation of GSK-3β-related motifs located in the N-terminal region of the molecule. One mechanism by which ASPH exerts its effector function is by activating downstream Notch signaling to promote cell migration and invasion.

### ASPH Overexpression in Human Tumors

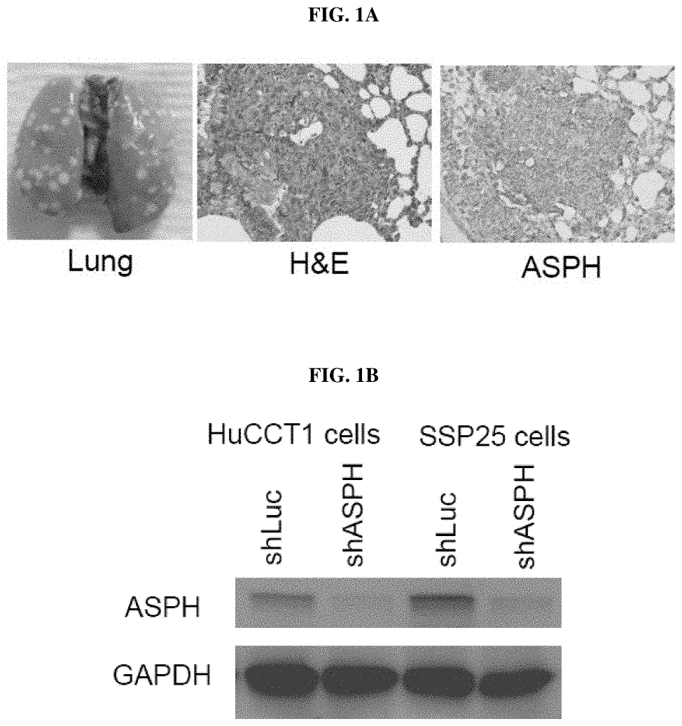

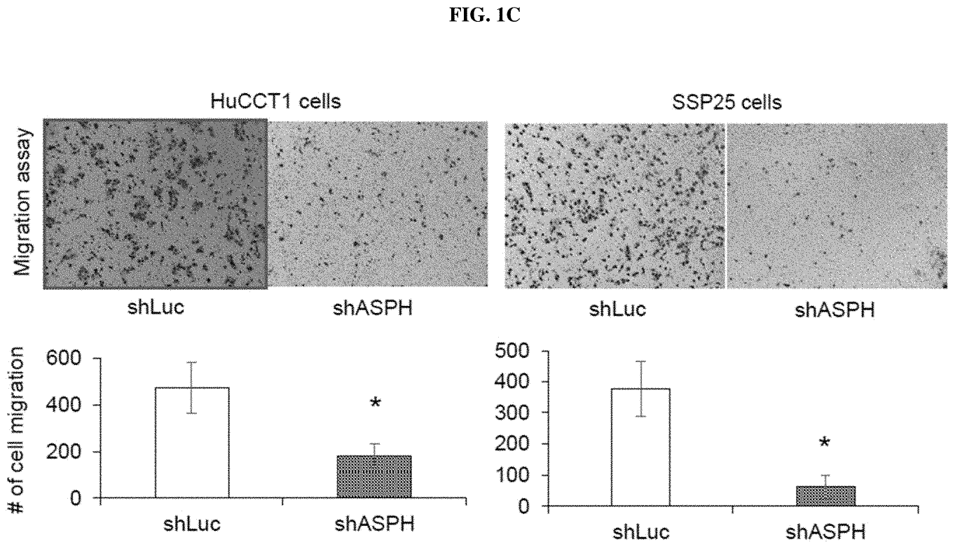

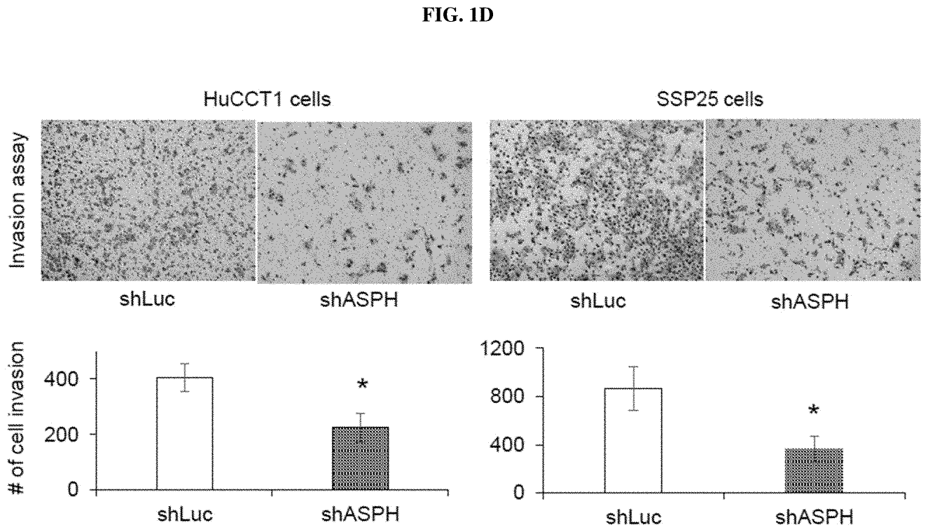

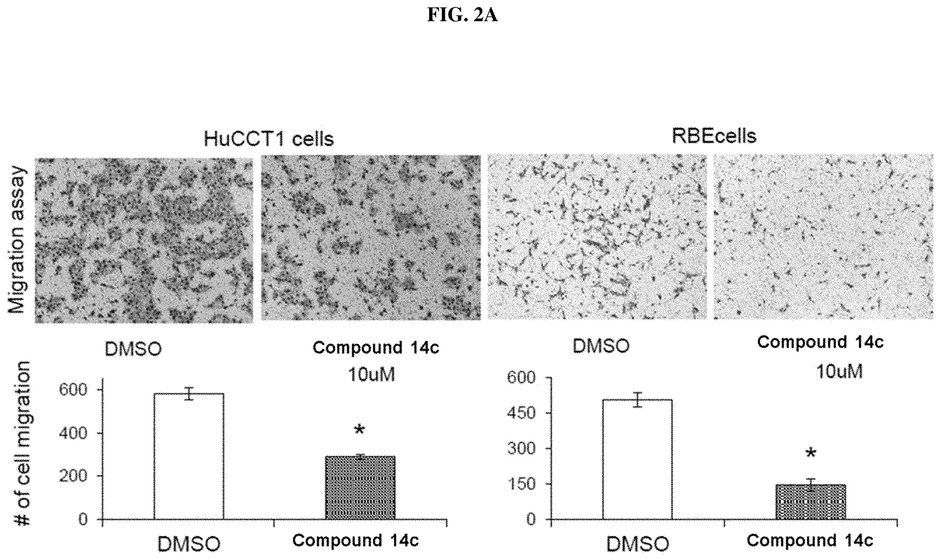

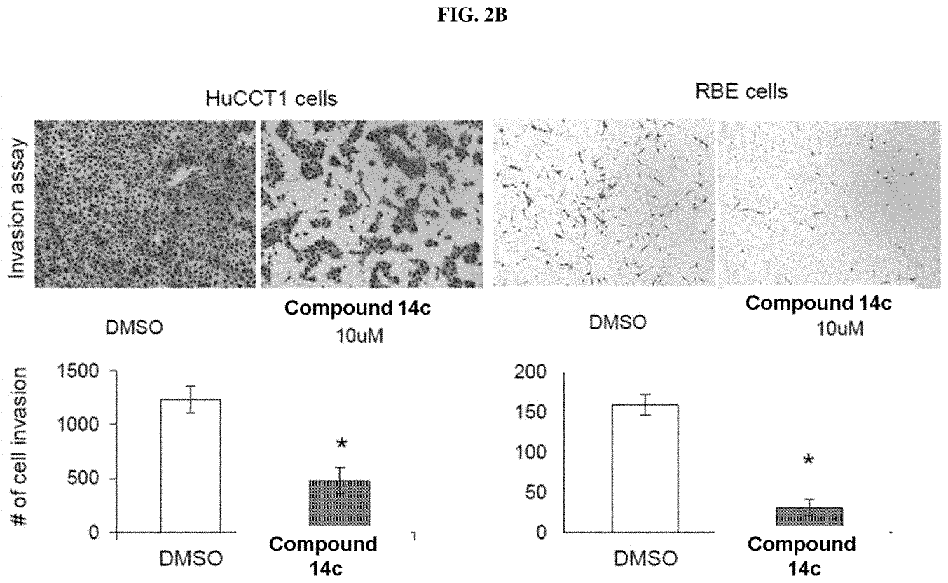

Table 2 details the overexpression of ASPH at the protein and RNA level as determined by IHS and qRT-PCR respectively in various human tumors indicating that it is a therapeutic target for a variety of human solid malignancies with a poor prognosis. FIGS. 1 and 2 show examples of ASPH protein expression by IHS.

TABLE 2
Overexpression of ASPH in Human Tumors
Compared to Normal Tissue by IHS and qRT-PCR

| | | Number | |
|---|---|---|---|
| Tumor type | Number | Positive | (%) |
| Pancreatic Cancer | 101 | 98 | (97%) |
| Hepatocellular Cancer | 95 | 87 | (92%) |
| Cholangiocarcinoma | 20 | 20 | (100%) |
| Lung | 16 | 16 | (100%) |
| Colon Cancer | 10 | 6 | (60%) |
| Breast Cancer | 17 | 17 | (100%) |
| Prostatic Cancer | 32 | 30 | (94%) |
| Glioblastoma | 5 | 5 | (100%) |

### Oncogenic Role of ASPH β-Hydroxylase Activity

The C-terminus of ASPH contains amino acid (AA) sequence of the catalytic site (M⁶⁷⁰HPGFH⁶⁷⁵) and its sequence is identical in human, rat, mouse, and cattle. The H⁶⁷⁵AA is specifically involved in Fe²⁺ coordination and critical for its enzymatic activity, also highly conserved in the chicken and fly. A H⁶⁷⁵R mutation reduces P-hydroxylase activity to <1% of wild-type protein while H⁶⁷⁵D reduces it to 20%. In this context the H⁶⁷⁵R mutant protein loses the ability to promote cell proliferation, motility, migration, invasion, colony formation in soft agar, as well as metastasis and tumor formation in nude mice compared to the “wild-type” sequence and it also can function as a dominant negative mutant to inhibit the function of the endogenous “Wild-Type” protein. These findings indicate that inhibition of β-hydroxylase activity promotes anti-tumor effects. The crystal structure of the catalytic site region has been elucidated and is available in the public database (RCSB protein database; code 3RCQ). Small molecule inhibitors for use as anti-tumor agents were identified by their ability to fit into the catalytic site region of ASPH and inhibit ASPH enzymatic activity.

### Generation of a Small Molecule Inhibitor (SMI) of ASPH Enzymatic Activity

Compounds of the present invention can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T.W., Wuts, P.G.M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art. The following descriptions of synthetic methods are designed to illustrate, but not to limit, general procedures for the preparation of compounds of the present invention.

### Therapeutic Uses of Compositions Comprising Compounds of the Invention

In some aspects, this invention provides for the use of a compound as herein described, or its isomer, metabolite, tautomer, pharmaceutically acceptable salt, pharmaceutical product, polymorph, crystal, N-oxide, hydrate, or any combination thereof, for treating, suppressing, preventing, reducing the severity, reducing the risk, or inhibiting a cell proliferation disorder in a subject.

### Pharmaceutical Compositions

Related aspects of the invention are directed to compositions, including pharmaceutical compositions, comprising the compounds of the invention, noted above. One aspect of the invention is directed to a pharmaceutical composition comprising at least one pharmaceutically acceptable excipient and a therapeutically effective amount of the compound or salt disclosed above. Still another aspect of the invention relates to a method for pharmaceutical formulation of previously described compounds for use in oral and intravenous applications, and in implantable materials.

Another aspect of the present invention relates to a pharmaceutical composition including a pharmaceutical composition can contain one or more of the above-identified compounds of the present invention.

Typically, the pharmaceutical composition of the present invention will include a compound of the present invention or its pharmaceutically acceptable salt, as well as a pharmaceutically acceptable carrier. The term “pharmaceutically acceptable carrier” refers to any suitable adjuvants, carriers, excipients, or stabilizers, and can be in solid or liquid form such as, tablets, capsules, powders, solutions, suspensions, emulsions, or implantable disc.

Typically, the composition will contain from about 0.01 to 99 percent, preferably from about 20 to 75 percent of active compound(s), together with the adjuvants, carriers and/or excipients. While individual needs may vary, determination of optimal ranges of effective amounts of each component is within the skill of the art. Typical dosages comprise about 0.01 to about 100 mg/kg body wt. The preferred dosages comprise about 0.1 to about 100 mg/kg body wt. The most preferred dosages comprise about 1 to about 100 mg/kg body wt. Treatment regimen for the administration of the compounds of the present invention can also be determined readily by those with ordinary skill in art. That is, the frequency of administration and size of the dose can be established by routine optimization, preferably while minimizing any side effects.

### Dosage Forms

The solid unit dosage forms can be of the conventional type. The solid form can be a capsule and the like, such as an ordinary gelatin type containing the compounds of the present invention and a carrier, for example, lubricants and inert fillers such as, lactose, sucrose, or cornstarch. In another embodiment, these compounds are tabulated with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

### Optional Coatings

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

### Excipients

For oral therapeutic administration, these active compounds can be incorporated with excipients and used in the form of tablets, capsules, elixirs, suspensions, syrups, and the like. Such compositions and preparations should contain at least 0.1% of active compound. The percentage of the compound in these compositions can, of course, be varied and can conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 mg and 800 mg of active compound.

### Modes of Administration

The active compounds of the present invention may be orally administered, for example, with an inert diluent, or with an assailable edible carrier, or they can be enclosed in hard or soft shell capsules, or they can be compressed into tablets, or they can be incorporated directly with the food of the diet.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds or pharmaceutical compositions of the present invention may also be administered in injectable dosages by solution or suspension of these materials in a physiologically acceptable diluent with a pharmaceutical adjuvant, carrier or excipient. Such adjuvants, carriers and/or excipients include, but are not limited to, sterile liquids, such as water and oils, with or without the addition of a surfactant and other pharmaceutically and physiologically acceptable components. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

The pharmaceutical forms suitable for implantable use include sterile wafers of polycarboxyphenoxypropane-sebacic-acid (pCPP:SA) polymers, poly(D,L-lactic acid), polyhydroxybutyrate, lysine diisocyanate (LDI)-glycerol polyurethane, and poly(D-L lactide-co-glycolide). In all cases, the form should be sterile and should be a wafer or disc of suitable dimensions for surgical implantation in the brain. The polymers should be stable under the conditions of manufacture and storage and should be preserved against the contaminating action of microorganisms, such as bacteria and fungi. The wafers should be biodegradable ranging from 24 hours up to 6 months.

In one aspect, the invention provides compounds and compositions, including any aspect described herein, for use in any of the methods of this invention. In one aspect, use of a compound of this invention or a composition comprising the same, will have utility in inhibiting, suppressing, enhancing or stimulating a desired response in a subject, as will be understood by one skilled in the art. In another embodiment, the compositions may further comprise additional active ingredients, whose activity is useful for the particular application for which the compound of this invention is being administered.

Pharmaceutical compositions or therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the modulators can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

The mode of administration may be oral, for intestinal delivery; intranasal, for nasal delivery; and intravenous for delivery through the blood-brain barrier. Other modes of administration as are known in the art may also be used, including, but not limited to intrathecal, intramuscular, intrabronchial, intrarectal, intraocular, and intravaginal delivery.

The modulator compounds can be administered as oral dosage compositions for small intestinal delivery. Such oral dosage compositions for small intestinal delivery are well-known in the art, and generally comprise gastroresistent tablets or capsules (Remington's Pharmaceutical Sciences, 16th Ed., Eds. Osol, Mack Publishing Co., Chapter 89 (1980); Digenis et al, J. Pharm. Sci., 83:915-921 (1994); Vantini et al, Clinica Terapeutica, 145:445-451 (1993); Yoshitomi et al, Chem. Pharm. Bull., 40:1902-1905 (1992); Thoma et al, Pharmazie, 46:331-336 (1991); Morishita et al, Drug Design and Delivery, 7:309-319 (1991); and Lin et al, Pharmaceutical Res., 8:919-924 (1991)); each of which is incorporated by reference herein in its entirety).

Tablets are made gastroresistent by the addition of compounds such as cellulose acetate phthalate or cellulose acetate terephthalate.

Capsules are solid dosage forms in which the tight junction modulator compound is enclosed in either a hard or soft, soluble container or shell of gelatin. The gelatin used in the manufacture of capsules is obtained from collagenous material by hydrolysis. There are two types of gelatin. Type A, derived from pork skins by acid processing, and Type B, obtained from bones and animal skins by alkaline processing. The use of hard gelatin capsules permit a choice in prescribing a tight junction modulator compound or a combination thereof at the exact dosage level considered best for the individual subject. The hard gelatin capsule consists of two sections, one slipping over the other, thus completely surrounding the tight junction modulator compound. These capsules are filled by introducing the modulator compound, or gastroresistent beads containing the modulator compound, into the longer end of the capsule, and then slipping on the cap. Hard gelatin capsules are made largely from gelatin, FD&C colorants, and sometimes an opacifying agent, such as titanium dioxide. The USP permits the gelatin for this purpose to contain 0.15% (w/v) sulfur dioxide to prevent decomposition during manufacture.

In the context of the present invention, oral dosage compositions for small intestinal delivery also include liquid compositions which contain aqueous buffering agents that prevent the modulator compound from being significantly inactivated by gastric fluids in the stomach, thereby allowing the modulator compound to reach the small intestines in an active form. Examples of such aqueous buffering agents which can be employed in the present invention include bicarbonate buffer (pH 5.5 to 8.7, preferably about pH 7.4).

When the oral dosage composition is a liquid composition, it is preferable that the composition be prepared just prior to administration so as to minimize stability problems. In this case, the liquid composition can be prepared by dissolving lyophilized tight junction modulator compound in the aqueous buffering agent. Oral dosage compositions for small intestinal delivery also include liquid compositions which may optionally contain aqueous buffering agents that prevent the therapeutic agent and tight junction modulator compound from being significantly inactivated by gastric fluids in the stomach, thereby allowing the biologically active ingredient and tight junction modulator compound to reach the small intestines in an active form. Examples of such aqueous buffering agents which can be employed in the present invention include bicarbonate buffer (pH 5.5 to 8.7, preferably about pH 7.4).

When the oral dosage composition is a liquid composition, it is preferable that the composition be prepared just prior to administration so as to minimize stability problems. In this case, the liquid composition can be prepared by dissolving lyophilized therapeutic agent and tight junction modulator compound in the aqueous buffering agent.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. For sterile powders used in the preparation of sterile injectable solutions, the preferred methods of preparation are vacuum drying and freeze-drying which yields a powder of the active ingredient plus any additional desired ingredient from a previously sterile-filtered solution thereof.

A “nasal” delivery composition differs from an “intestinal” delivery composition in that the latter must have gastroresistent properties in order to prevent the acidic degradation of the active agents in the stomach, whereas the former generally comprises water-soluble polymers with a diameter of about 50 11 m in order to reduce the mucociliary clearance, and to achieve a reproducible bioavailability of the nasally administered agents.

An “intravenous” delivery composition differs from both the “nasal” and “intestinal” delivery compositions in that there is no need for gastroresistance or water-soluble polymers in the “intravenous” delivery composition.

Nasal dosage compositions for nasal delivery are well-known in the art. Such nasal dosage compositions generally comprise water-soluble polymers that have been used extensively to prepare pharmaceutical dosage forms (Martin et al, In: Physical Chemical Principles of 20 Pharmaceutical Sciences, 3rd Ed., pages 592-638 (1983)) that can serve as carriers for peptides for nasal administration (Davis, In: Delivery Systems for Peptide Drugs, 125:1-21 (1986)). The nasal absorption of pap tides embedded in polymer matrices has been shown to be enhanced through retardation of nasal mucociliary clearance (Illum et al, Int. J. Pharm., 46:261-265 (1988É). Other possible enhancement mechanisms include an increased concentration gradient or 25 decreased diffusion path for peptides absorption (Ting et al, Pharm. Res., 9:1330-1335 (1992). However, reduction in mucociliary clearance rate has been predicted to be a good approach toward achievement or reproducible bioavailability of nasally administered systemic drugs (Gonda et al, Pharm. Res., 7:69-75 (1990)). Microparticles with a diameter of about 50 p m are expected to deposit in the nasal cavity (Bjork et al, Int. J. Pharm., 62:187-192 (1990); and lllum et al, Int. J. Pharm., 39:189-199 (1987), while microparticles with a diameter under 10 pm can escape the filtering system of the nose and deposit in the lower airways. Microparticles larger than 200 p m in diameter will not be retained in the nose after nasal administration (Lewis et al, Proc. Int. Symp. Control Re. Bioact. Mater., 17:280-290 (1990)).

The particular water-soluble polymer employed is not critical to the present invention, and can be selected from any of the well-known water-soluble polymers employed for nasal dosage forms. A typical example of a water-soluble polymer useful for nasal delivery is polyvinyl alcohol (pvA). This material is a swellable hydrophilic polymer whose physical properties depend on the molecular weight, degree of hydrolysis, cross-linking density, and crystallinity (Peppas et al, In: Hydrogels in Medicine and Pharmacy, 3:109-131 (1987). PYA can be used in the coating of dispersed materials through phase separation, spray-drying, spray-embedding, and spray-densation (Ting et al, supra).

A “skin” delivery composition comprising a modulator compound of the invention may include in addition a therapeutic or immunogenic agent, fragrance, creams, ointments, colorings, and other compounds so long as the added component does not deleteriously affect transdermal delivery of the therapeutic or immunogenic agent. Conventional pharmaceutically acceptable emulsifiers, surfactants, suspending agents, antioxidants, osmotic enhancers, extenders, diluents and preservatives may also be added. Water soluble polymers can also be used as carriers.

The particular therapeutic or immunogenic agent employed is not critical to the present invention, and can be, e.g., any drug compound, biologically active peptide, vaccine, or any other moiety otherwise not absorbed through the transcellular pathway, regardless of size or charge.

The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically 35 discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

As used herein “pharmaceutically-acceptable carrier” includes any and all solvents, dispersion media, coatings, antibacterial and antifungal agents, isotonic and absorption delaying agents, and the like that are physiologically compatible. In one embodiment, the carrier is 10 suitable for parenteral administration. A carrier may be suitable for administration into the central nervous system (e.g., intraspinally or intracerebrally). Alternatively, the carrier can be suitable for intravenous, intraperitoneal or intramuscular administration. In another embodiment, the carrier is suitable for oral administration. Pharmaceutically-acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

While specific aspects of the invention have been described in detail, it will be appreciated by those skilled in the art that various modifications and alternatives to those details could be developed in light of the overall teachings of the disclosure. Accordingly, the particular arrangements disclosed are meant to be illustrative only, and not limiting as to the scope of the invention, which is to be given the full breadth of the appended claims, and any equivalent, thereof.

### Terms and Definitions

The following is a list of abbreviations, plus terms and their definitions, used throughout the specification and the claims:

General abbreviations and their corresponding meanings include: aa or AA=amino acid; mg=milligram(s); ml or mL=milliliter(s); mm=millimeter(s); mM=millimolar; nmol=nanomole(s); pmol=picomole(s); ppm=parts per million; RT=room temperature; U=units; ug, μg=micro gram(s); ul, μl=micro liter(s); uM, μM=micromolar, TEA=triethylamine, LDA=lithium diisopropyl amine, THF=tetrahydrofuran, DMAP=4-dimethylaminopyridine, DMF=N,N′-dimethylformamide.

The terms “cell” and “cells”, which are meant to be inclusive, refer to one or more cells which can be in an isolated or cultured state, as in a cell line comprising a homogeneous or heterogeneous population of cells, or in a tissue sample, or as part of an organism, such as an insect larva or a transgenic mammal.

The term “amino acid” encompasses both naturally occurring and non-naturally occurring amino acids unless otherwise designated.

The term “an effective amount” means an amount of the substance in question which produces a statistically significant effect. For example, an “effective amount” for therapeutic uses is the amount of the composition comprising an active compound herein required to provide a clinically significant alteration in a measurable trait. Such effective amounts will be determined using routine optimization techniques and are dependent on the particular condition to be treated, the condition of the patient, the route of administration, dosage required for the compounds of the invention is manifested as that which induces a statistically significant difference between treatment and control groups.

A “therapeutically effective amount” refers to an amount effective, at dosages and for periods of time necessary, to achieve the desired therapeutic result. A therapeutically effective amount of modulator may vary according to factors such as the disease state, age, sex, and weight of the individual, and the ability of the modulator to elicit a desired response in the individual. Dosage regimens may be adjusted to provide the optimum therapeutic response. A therapeutically-effective amount is also one in which any toxic or detrimental effects of the modulator are outweighed by the therapeutically beneficial effects.

A “prophylactically effective amount” refers to an amount effective, at dosages and for periods of time necessary, to achieve the desired prophylactic result. A prophylactically effective amount can be determined as described above for the therapeutically-effective amount. Typically, since a prophylactic dose is used in subjects prior to or at an earlier stage of disease, the prophylactically effective amount will be less than the therapeutically-effective amount.

As used herein, the term “cell proliferative disorder” refers to conditions in which unregulated or abnormal growth, or both, of cells can lead to the development of an unwanted condition or disease, which may or may not be cancerous. Exemplary cell proliferative disorders that may be treated with the compounds of the invention encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. A cell proliferative disorder includes a precancer or a precancerous condition. A cell proliferative disorder includes cancer. The methods and uses provided herein can be or may be used to treat or alleviate a symptom of cancer or to identify suitable candidates for such purposes.

The term “cancer” includes solid tumors, as well as, hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

As used herein, “treating” or “treat” describes the management and care of a patient for the purpose of combating a disease, condition, or disorder and includes the administration of a compound of the present invention, or a pharmaceutically acceptable salt, prodrug, metabolite, polymorph or solvate thereof, to alleviate the symptoms or complications of a disease, condition or disorder, or to eliminate the disease, condition or disorder. The term “treat” can also include treatment of a cell in vitro or an animal model.

A compound of the present invention, or a pharmaceutically acceptable salt, prodrug, metabolite, polymorph or solvate thereof, can or may also be used to prevent a relevant disease, condition or disorder, or used to identify suitable candidates for such purposes. As used herein, “preventing,” “prevent,” or “protecting against” describes reducing or eliminating the onset of the symptoms or complications of such disease, condition or disorder.

As used herein, the term “alleviate” is meant to describe a process by which the severity of a sign or symptom of a disorder is decreased. Importantly, a sign or symptom can be alleviated without being eliminated. The administration of pharmaceutical compositions of the invention can or may lead to the elimination of a sign or symptom, however, elimination is not required. Effective dosages should be expected to decrease the severity of a sign or symptom. For instance, a sign or symptom of a disorder such as cancer, which can occur in multiple locations, is alleviated if the severity of the cancer is decreased within at least one of multiple locations.

As used herein, a “subject” is interchangeable with a “subject in need thereof”, both of which refer to a subject having a cell proliferation disorder, or a subject having an increased risk of developing such disorder relative to the population at large. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human. A subject in need thereof can be one who has been previously diagnosed or identified as having cancer or a precancerous condition. A subject in need thereof can also be one who has (e.g., is suffering from) cancer or a precancerous condition. Alternatively, a subject in need thereof can be one who has an increased risk of developing such disorder relative to the population at large (i.e., a subject who is predisposed to developing such disorder relative to the population at large). A subject in need thereof can have a precancerous condition. The term “animal” includes human beings.

The term “optionally substituted” moiety refers to either unsubstituted chemical moiety (e.g., alkyl, aryl, heteroaryl, etc.) or a chemical moiety having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “substituted aryl or heteroaryl” refers to aromatic or heteroaromatic rings may contain one or more substituents such as —OH, SH, —CN, —F, —Cl, —Br, —R, —NO₂—NO, —NH2, —NHR, —NRR, —C(O)R, —C(O)OH, —C(O)OR, —C(O)NH2, —C(O)NHR, —C(O)NRR, and the like where each R is independently (C₁-C₅) alkyl, substituted (C₁-C₆) alkyl, (C₂-C₆) alkenyl, substituted (C₂-C₆) alkenyl, (C₂-C₆) alkynyl, substituted (C₂-C₆) alkynyl, (C₅-C₂) aryl, substituted (C₅-C₂) aryl, (C₆-C₂₆) arylalkyl, substituted (C₆-C₂₆) arylalkyl, 5-20 membered heteroaryl, substituted 5-20 membered heteroaryl, 6-26 membered heteroarylalkyl or substituted 6-26 membered heteroarylalkyl.

An “arylalkyl” or an “aralkyl” moiety is an alkyl substituted with an aryl (e.g., phenylmethyl (benzyl)). An “alkylaryl” moiety is an aryl substituted with an alkyl (e.g., methylphenyl).

A “derivative” of a compound X (e.g., a peptide or amino acid) refers to a form of X in which one or more reactive groups on the compound have been derivatized with a substituent group. Peptide derivatives include pep tides in which an amino acid side chain, the peptide backbone, or the amino’ or carboxy-terminus has been derivatized (e.g., peptidic compounds with 5 methylated amide linkages).

An “analogue” of a compound X refers to a compound which retains chemical structures of X necessary for functional activity of X yet which also contains certain chemical structures which differ from X. An analogue of a naturally-occurring peptide, is a peptide which includes one or more non-naturally-occurring amino acids.

The term “mimetic refers to a compound having similar functional and/or structural properties to another known compound or a particular fragment of that known compound. A “mimetic” of a compound X refers to a compound in which chemical structures of X necessary for functional activity of X have been replaced with other chemical structures which mimic the conformation of X. The term mimetic, and in particular, peptidomimetic, is intended to include isosteres.

The term “cyclic group”, as used herein, is intended to include cyclic saturated or unsaturated (i.e., aromatic) group having from about 3 to 10, preferably about 4 to 8, and more preferably about 5 to 7, carbon atoms. Exemplary cyclic groups include cyclopropyl, cyclobutyl, cyclopentyl, cyclohexyl, and cyclooctyl. Cyclic groups may be unsubstituted or substituted at one or more ring positions. Thus, a cyclic group may be substituted with halogens, alkyls, cycloalkyls, alkenyls, alkynyls, aryls, heterocycles, hydroxyls, aminos, nitros, thiols amines, imines, amides, phosphonates, phosphines, carbonyls, carboxyls, silyls, ethers, thioethers, sulfonyls, sulfonates. selenoethers, ketones, aldehydes, esters, ′CF₃, ′CN, or the like.

The term “heterocyclic group” is intended to include cyclic saturated or unsaturated (i.e., aromatic) group having from about 3 to 10, preferably about 4 to 8, and more preferably about 5 to 7, carbon atoms, wherein the ring structure includes about one to four heteroatoms. Heterocyclic groups include pyrrolidine, oxolane, thiolane, imidazole, oxazole, piperidine, piperazine, morpholine and pyridine. The heterocyclic ring can be substituted at one or more positions with such substituents as, for example, halogens, alkyls, cycloalkyls, alkenyls, alkynyls, aryls, other heterocycles, hydroxyl, amino, nitro thiol, amines, imines, amides, phosphonates, phosphines, carbonyls, carboxyls, eilyls, ethers, thioethers, sulfonyls, selenoethers, ketones, aldehydes, esters, CF₃, CN, or the like. Heterocycles may also be bridged or fused to other cyclic groups as described below.

“Aryl” includes groups with aromaticity, including “conjugated,” or multicyclic systems with at least one aromatic ring and do not contain any heteroatom in the ring structure. Examples include phenyl, benzyl, 1,2,3,4-tetrahydronaphthalenyl, etc.

“Heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, naphthrydine, indole, benzofuran, purine, benzofuran, deazapurine, indolizine.

In the case of multicyclic aromatic rings, only one of the rings needs to be aromatic (e.g., 2,3-dihydroindole), although all of the rings may be aromatic (e.g., quinoline). The second ring can also be fused or bridged.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl).

The term “polycyclic group” as used herein is intended to refer to two or more saturated or unsaturated (i.e., aromatic) cyclic rings in which two or more carbons are common to two adjoining rings, e.g., the rings are “fused rings”. Rings that are joined through non-adjacent atoms are termed “bridged” rings. Each of the rings of the polycyclic group can he substituted with such substituents as described above, as for example, halogens, alkyls, cycloalkyls, alkenyls, alkynyls, hydroxyl, amino, nitro, thiol, amines, imines, amides, phosphonates, phosphines, carbonyls, carboxyls, silyls, ethers, thioethers, sulfonyls, selenoethers, ketones, aldehydes, esters, CF₃, CN, or the like.

As used herein, the term “modulating group” and “modifying group” are used interchangeably to describe a chemical group directly or indirectly attached to a peptidic structure. For example, a modifying group(s) can be directly attached by covalent coupling to the peptidic structure or a modifying group(s) can be attached indirectly by a stable non-covalent association.

The compounds described herein include the compounds themselves, as well as their salts, their solvates, and their prodrugs, if applicable. A salt, for example, can be formed between an anion and a positively charged group (e.g., amino) on a substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt.

Likewise, a salt can also be formed between a cation and a negatively charged group (e.g., carboxylate) on a substituted benzene compound. Suitable cations include sodium ion, potassium ion, magnesium ion, calcium ion, and an ammonium cation such as tetramethylammonium ion. The substituted benzene compounds also include those salts containing quaternary nitrogen atoms.

Examples of prodrugs include esters and other pharmaceutically acceptable derivatives, which, upon administration to a subject, are capable of providing active substituted benzene compounds.

Additionally, the compounds of the present invention, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

In the present specification, the structural formula of the compound represents a certain isomer for convenience in some cases, but the present invention includes all isomers, such as geometrical isomers, optical isomers based on an asymmetrical carbon, stereoisomers, tautomers, and the like, it being understood that not all isomers may have the same level of activity. In addition, a crystal polymorphism may be present for the compounds represented by the formula. It is noted that any crystal form, crystal form mixture, or anhydride or hydrate thereof is included in the scope of the present invention. Furthermore, so-called metabolite which is produced by degradation of the present compound in vivo is included in the scope of the present invention.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. An example of ketone-enol tautomerism are as shown below.

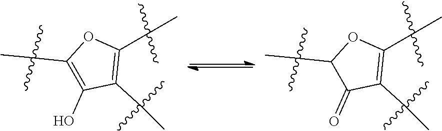 [SMILES: *c1oc(*)c(O)c1* ; *C1=C(*)C(=O)C(*)O1]

A small molecule is a compound that is less than 2000 Daltons in mass. The molecular mass of the small molecule is preferably less than 1000 Daltons, more preferably less than 600 Daltons, e.g., the compound is less than 500 Daltons, 400 Daltons, 300 Daltons, 200 Daltons, or 100 Daltons.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Compounds such as small molecule inhibitors, polynucleotides, polypeptides, or other agents are purified and/or isolated. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. An “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified also defines a degree of sterility that is safe for administration to a human subject, e.g., lacking infectious or toxic agents.

### EXAMPLES

The foregoing discussion may be better understood in connection with the following representative examples which are presented for purposes of illustrating the principle methods and compositions of the invention, and not by way of limitation. Various other examples will be apparent to the person skilled in the art after reading the present disclosure without departing from the spirit and scope of the invention. It is intended that all such other examples be included within the scope of the appended claims.

### Example 1: ASPH Inhibitors Reduce Tumor Growth or Metastasis

Cholangiocarcinoma is a devastating disease with a 2% 5-year survival if the disease metastases outside the liver. Thus, there is a need to develop effective therapies for cholangiocarcinoma. Understanding how cholangiocarcinoma metastasizes has identified therapeutic targets. The aspartate beta-hydroxylase (ASPH) has been demonstrated to be highly expressed in cholangiocarcinoma but not in normal bile ducts, and found to stimulate tumor cell migration. Thus, this study aimed to clarify the role of ASPH during bile duct oncogenesis. The analysis was performed in vitro and in vivo preclinical animal model by using molecular and biochemical strategies to regulate ASPH expression and function. Knockdown of ASPH substantially inhibited cell migration and invasion in two cholangiocarcinoma cell lines. Targeting ASPH with a small molecule inhibitor (SMI) suppressed cholangiocarcinoma growth and progression. Molecular mechanism studies demonstrated that knockdown of ASPH suppressed protein levels of the matrix metalloproteinases. Treatment with specific inhibitors for matrix metalloproteinases suppressed tumor cell migration in control but not in ASPH knockdown cholangiocarcinoma cells, suggesting that ASPH modulates cholangiocarcinoma metastasis by regulating matrix metalloproteinases. Furthermore, using an ASPH inhibitor in a rat cholangiocarcinoma model established with BED-Neu-CL#24 cholangiocarcinoma cells, it was found that targeting ASPH inhibited intrahepatic cholangiocarcinoma metastasis and expression of matrix metalloproteinases. ASPH modulates cholangiocarcinoma metastasis via matrix metalloproteinases. Targeting ASPH expression and function inhibits intrahepatic cholangiocarcinoma metastasis.

Cholangiocarcinoma (CCA) is a highly lethal disease with a low 5-year survival rate of 2%[1]. Recent whole genomic sequencing has identified several new mutations in IDH1 gene and fibroblast growth factor receptor 2 (FGFR2) generated fusion protein in 20% and 16% of CCAs, respectively [2-4]. These findings have led to the development of inhibitors for both mutations and these agents have been proposed for evaluation in clinical trials. In view of the potential of these inhibitors, there are still approximately 80% of patients without an effective therapeutic approach.

The high metastatic potential of CCA usually portends a grim prognosis. The presence of metastasis is one of the critical determinants for deciding the best therapeutic approach for this disease. Although surgical resection remains the first line of therapy, most patients with metastatic spread are not candidates for surgery. However, chemotherapy with agents, such as gemcitabine and cisplatin, are often used for patients with unresectable tumors. Thus, the use of systemic chemotherapy is still associated with a poor prognosis. The high metastatic potential of CCA usually leads to a grim prognosis. The presence of metastasis is one of the critical determinants for choosing the appropriate therapeutic approach for CCA patients. Although surgical resection is considered an effective treatment, patients with metastasis are normally not candidates for it. Thus, chemotherapies, such as gemcitabine and cisplatin, are generally considered for CCA patients with unresectable tumors. But systemic chemotherapies in CCA patients still has a very poor prognosis. Therefore, understanding the molecular pathogenesis of CCA growth, development and spread could allow for tumor resection in some patients.

Aspartate beta-hydroxylase (ASPH) is an alpha-ketoglutarate dependent enzyme highly expressed in CCA but not in healthy bile duct cells. Its function is to hydroxylate epidermal growth factor-like domains that are present in certain proteins such as Notch1 and TGFβ1; both agents have been shown previously to participate in CCA malignant progression. ASPH was found to physically interact with Jagged 1 and notch 1. These interactions may promote Notch1 signaling which results in metastatic spread. It has also been observed that ASPH expression is correlated with patient prognosis. Targeting ASPH with antisense oligodeoxynucleotide and shRNA, have resulted in a reduction of tumor cell motility in vitro. Although ASPH has been found to be an important factor for malignant progression in vitro, prior to the invention, it was not clear if targeting ASPH with molecular and pharmacological approaches will inhibit metastatic spread in vivo.

In the present study, involvement of ASPH in metastasis was evaluated by determining tumor cell migration and invasion in vitro, and more importantly, in vivo using a rat CCA metastatic model. ASPH was found to be required for tumor cell migration and invasion. Since the matrix metalloproteinases are involved in metastasis, targeting ASPH levels and function suppressed the expression of MMP [1, 2, 9, and 14]. Treatment of CCA cells with an inhibitor of MMPs, as well as ASPH knockdown, inhibited tumor cell migration. These results indicate that ASPH modulates CCA migration and invasion via regulating the activity of the MMPs. To further validate these findings, we determined the impact of ASPH expression and function on CCA metastasis in vivo. Inhibiting ASPH enzymatic activity was found to significantly reduced metastatic spread. Such results confirm that ASPH is required for promoting CCA metastasis and indicate that ASPH is a useful therapeutic target.

### Cell Culture

Human cholangiocarcinoma cell lines, HuCCT1, RBE, and SSP25 were purchased from the RIKEN cell line bank. They were cultured in RPMI-1640 medium supplemented with 10% (v/v) fetal bovine serum, 2 mM of L-glutamine, and penicillin/streptomycin. A BDE-Neu cell line and BDE-Neu CL24 were cultured in Dulbecco's modified Eagle's medium supplemented with 10% (v/v) fetal bovine serum, 2 mM of L-glutamine, and penicillin/streptomycin. Plasmids pLKO.1-shRNA-luciferase (shLuc) and pLKO.1-shRNA-ASPH (shASPH) were purchased from Sigma-Aldrich (St. Louis, Mo.).

### Antibodies

The anti-ASPH polyclonal antibody was produced in rabbits by administering recombinant human LSD2 protein. Anti-MMP1 (ab38927), anti-MMP2 (ab110186), anti-MMP9 (ab38906), and anti-MMP14 (ab3644) antibodies were purchased from Abcam (Cambridge, Mass.). The GAPDH antibody was purchased from Santa Cruz Biotechnology (Dallas, Tex.).

### Characterization of Small Molecule Inhibitors (SMIs) for ASPH

SMIs for targeting ASPH beta-hydroxylase activity were developed. A computer-generated drug design used the crystal structure of the ASPH catalytic site to derive several parent compounds and derivatives that were likely to fit into the catalytic site and inhibit enzymatic activity. Compound 14c is the third generation ASPH inhibitor developed by this method. (https://patents.justia.com/patent/20180237427).

### Cell Migration and Invasion Assay

The cell migration assay was performed using transwell chambers (pore size: 8 μm, Corning 3422) coated with Matrigel® (Corning 354480). The cell invasion assay was performed by using transwell chambers coated with Matrigel® (Corning 354480) as previously described. In brief, 10⁵ cells in 200 μl of serum-free medium were plated in the top chamber and 600 μl of culture medium containing 10% fetal bovine serum was preparedapplied in the lower chamber to stimulate cell for migration. Cells were incubated for 24 hours, fixed with methanol, stained with 10% Giemsa solution, and counted using ImageJ software (National Institute of Health, USA). The results of invasion assay were analyzed as described in the migration assay.

### Western Blotting

Western blotting (WB) was performed as description [20]. In brief, 50 μg of total protein was used for all WB analysis. After SDS page resolution, proteins were transferred to the PVDF membrane. The PVDF membrane was blocked with 5% non-fat milk prepared in 0.05% TBST for 1 hour and then hybridized with the 1st antibody at 4° C. overnight. Next, the 2nd antibody was used by incubation of the PVDF membrane for 1 hour. The PVDF membrane was washed with 0.05% TBST three times and exposed to the ECL substrate kit.

### Immunohistochemistry Staining

The immunohistochemistry assay was performed as known in the art. Tissue slides were de-paraffined, re-hydrated, and then subjected to antigen-retrieval using the antigen unmasking solution (Vector Inc., H3300). The endogenous peroxidase activity was quenched with 3% H2O2 prepared in methanol. Tissues slides were further blocked with a 5% milk and BSA solution prepared in 1×PBST for 1 hour. The slides were incubated with ASPH, MMP2, and MMP14 antibodies prepared in with 3% BSA in PBS) overnight at 4° C. overnight. The slides were washed with 0.05% TBST three times and then incubated with 1:200 dilution of biotinylated secondary antibody (Vector Laboratories) and the ABC solution (Vector Laboratories) at 20° C. The slides were incubated with DAB substrate to detect positive staining. Subsequently, the slides were dehydrated and mounted with PERMOUNT medium.room temperature. Images were taken using a Nikon microscope and analyzed with Image Pro Plus.

### Animal Studies

The intrahepatic CCA metastatic models weremetastasis model was established with BDE-Neu-CL24 cells. Rats were anesthetized with isoflurane, the abdomen was opened, and the liver inoculated with 3×10⁶ BDE-Neu-CL24 cells. The bile duct was then ligated following administration of 0.03 mg/kg of buprenorphine prior to surgery, and four hours after surgery, and the following morning for analgesia as needed. The rats were monitored daily and evaluated for weight change, body condition, and signs of ascites. The oral formulated compound 14c were prepared in capsules at a dose of 10 mg/kg and administered with an oral syringe. The rats were euthanized 18 days post-surgery with Isoflurane and cervical dislocation. Tumors were dissected away from the adjacent liver and weighed. All lung tissues were immersed in Bouin's fixative solution and the metastatic nodules on the surface of the lungs were counted.

### Statistical Analysis

Statistical analyses of two groups were done by Student's t-test unless otherwise stated. Equality of variance was examined using F-test. Mann-Whitney U test was used to assess the significance between the two groups with respect to the numbers of metastatic nodules in the rat lung by using the software “R”.

### ASPH Overexpression Modulates Cholangiocarcinoma Cell Migration

Previous studies have suggested that enhanced The ASPH expression correlates with lower survival rates and intrahepatic and lymph node metastases in hepatocellular carcinomas (HCC) and CCAs. The level of ASPH expression was evaluated in CCA metastatic tumors. The ASPH was found to be highly expressed in the metastatic nodules of the lung in the rat model (FIG. 1A). To further explore the function of ASPH in CCA metastatic spread, we suppressed expression in HuCCT1 and SSP25 human CCA cells using a lentivirus transduction system (FIG. 1B) and performed in vitro metastasis experiments using a Boyden chamber transwell assay. The migration assay demonstrated that knockdown of ASPH substantially reduced CCA cell motility (FIG. 1C). This result was further validated by an invasion assay using transwell inserts coated with extracellular matrix (ECM) material. The results indicate that knockdown of ASPH also significantly repressed cell invasive activity (FIG. 1D). It was observed that knockdown of ASPH substantially inhibited cell growth at day 3 and 5 by using MTT assay. As knockdown of ASPH did not reduce cell growth within 24 hours of seeding, the identified down-regulation in migration and invasion could be attributed to reduced capacity for CCA metastatic spread but not cell growth. Taken together, these results indicate that ASPH is involved in CCA mobility and targeting ASPH with specific inhibitors of enzymatic activity may reduce metastatic spread to the lung.

### ASPH Overexpression Modulates Cholangiocarcinoma Cell Migration

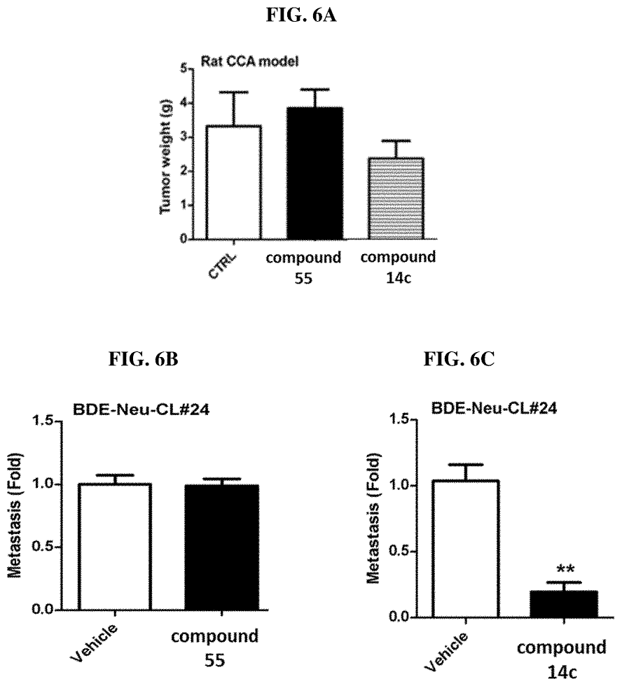

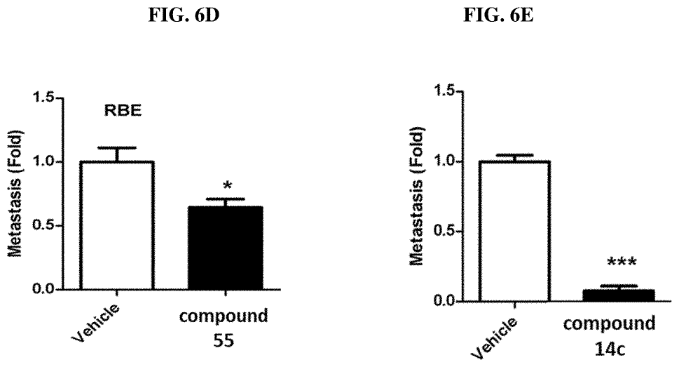

The compound 14c is the third generation ASPH small molecule inhibitor (SMI) with improved water solubility and inhibitory effects on ASPH activity as compared to the previous 1st and 2nd generations of ASPH SMIs which include MO-I-1100 and compound 55. Targeting ASPH with compound 14c both in vitro and was used in vivo demonstrated improved effects on CCA progression as compared to compound 55 (FIG. 6A). To determine if targeting ASPH with compound 14c suppresses CCA migration, we challenged RBE and HuCCT1 CCA cell lines with this agent. As expected, cell migration of both cell lines (FIG. 2A) were substantially suppressed with an observed reduction of 70% in RBE and 50% in HuCCT1 cells (FIG. 2A, lower panel). For the invasion assay, this SMI had even better suppressive effects with a 60% inhibition in HuCCT1 and 83% inhibition in RBE cell invasion capacity (FIG. 2B). These observations suggest that the enzymatic activity of ASPH is necessary for modulating CCA metastasis and may be a critical factor in disease progression 22 steps finely tuning cancer invasion.

### Matrix Metalloproteinases (MMPs) are Involved in ASPH-Mediated CCA Metastasis

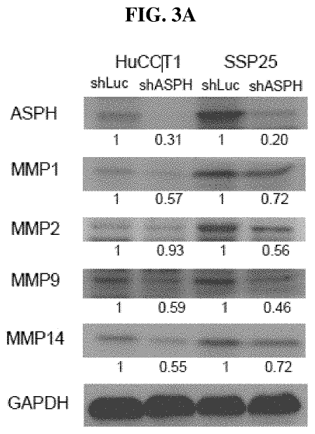

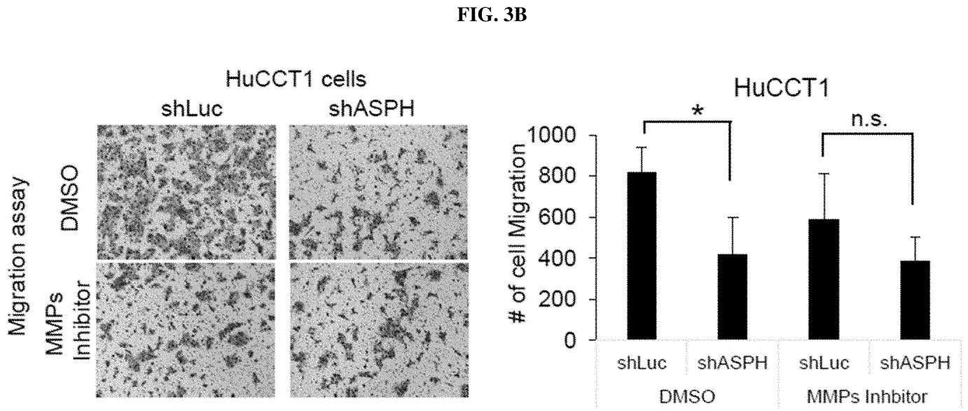

To determine how ASPH affects CCA migration, we examined if knockdown of ASPH inhibits CCA migration and invasion by affecting the expression of MMPs. Knockdown of ASPH suppressed the protein expression of MMP and found that inhibiting ASPH down-regulates MMP1, MMP2, MMP9, and MMP14. (FIG. 3A). To determine if ASPH modulates CCA migration by affecting MMP expression, we examined the effect of knockdown on CCA cell migration in two human cell lines. Knockdown of ASPH reduced the expression As shASPH-mediated down-regulation of MMPs (MMP1, MMP2, MMP9 and MMP14) and is consistent with the hypothesis that the MMPs are downstream effector of tumor cell migration. (FIGS. 3B and 3C).and decreased migration in CCA cells may be parallel events rather than a consequent outcome, we decided to examine if challenging shLuc and shASPH CCA cells with MMPs inhibitor reverses the suppressive effects of shASPH on CCA migration. Interestingly, MMPs inhibitor significantly reversed the suppressive effects of shASPH on HuCCT1 cells but to a less extent in SSP25 cells (FIGS. 3B and 3C), indicating a functional cooperation of ASPH and MMPs in CCA cell migration ability.

### ASPH Inhibitor Suppresses CCA Metastasis in a Rat Intrahepatic Tumor Model

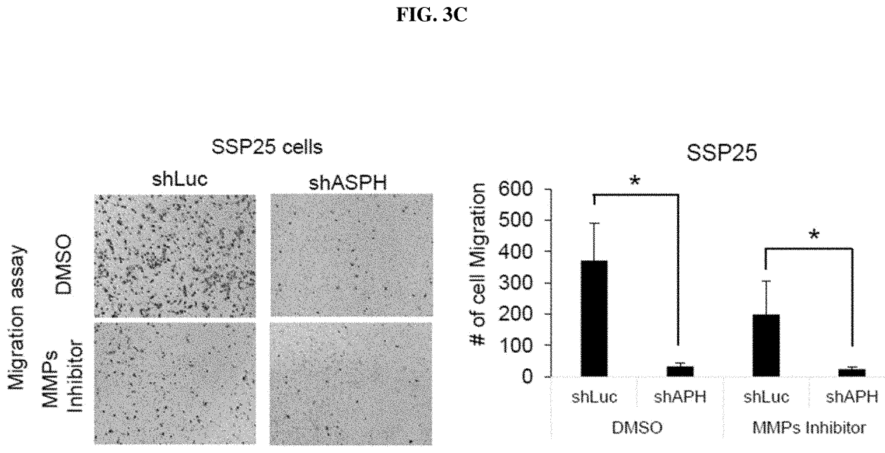

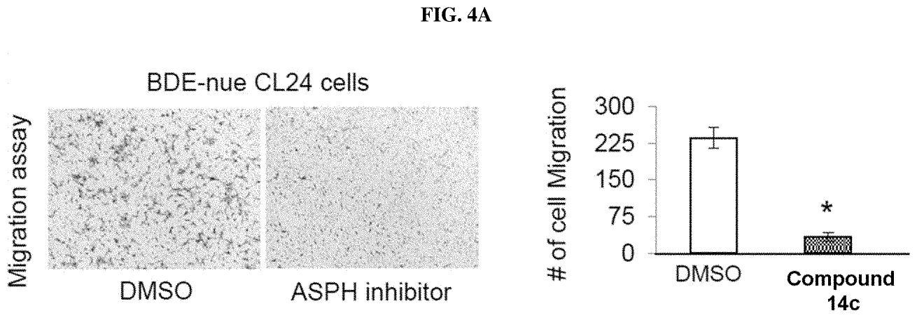

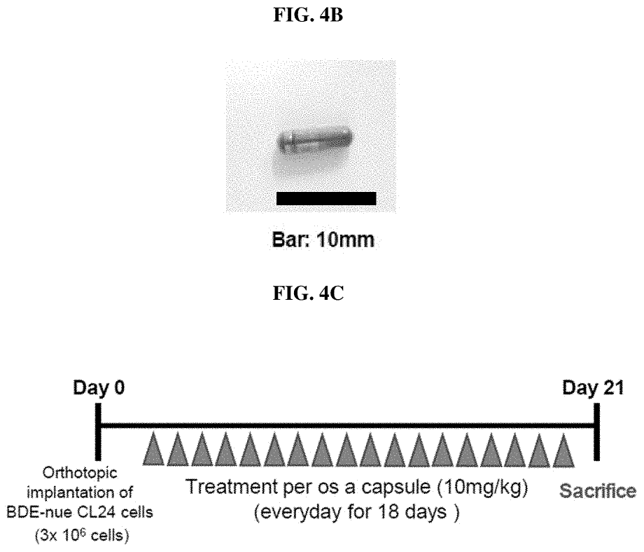

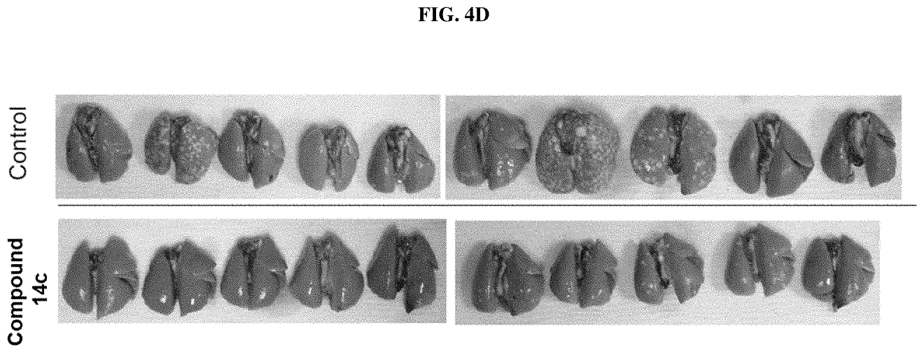

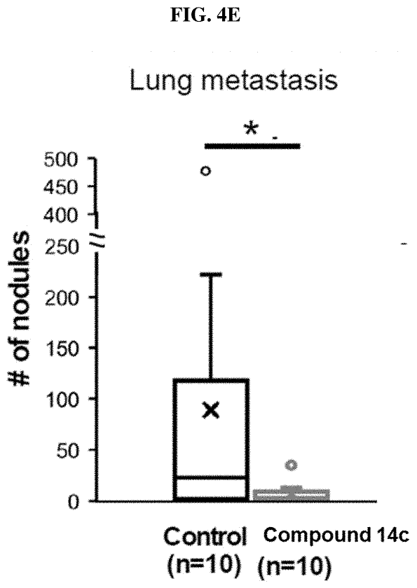

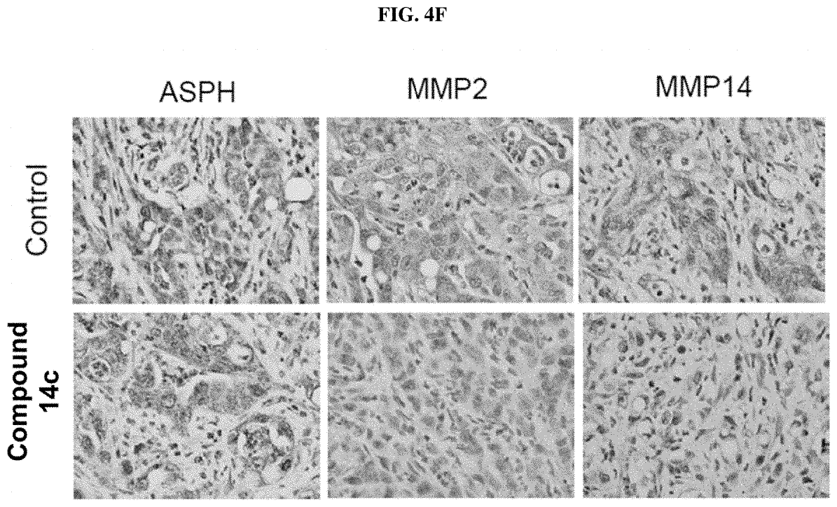

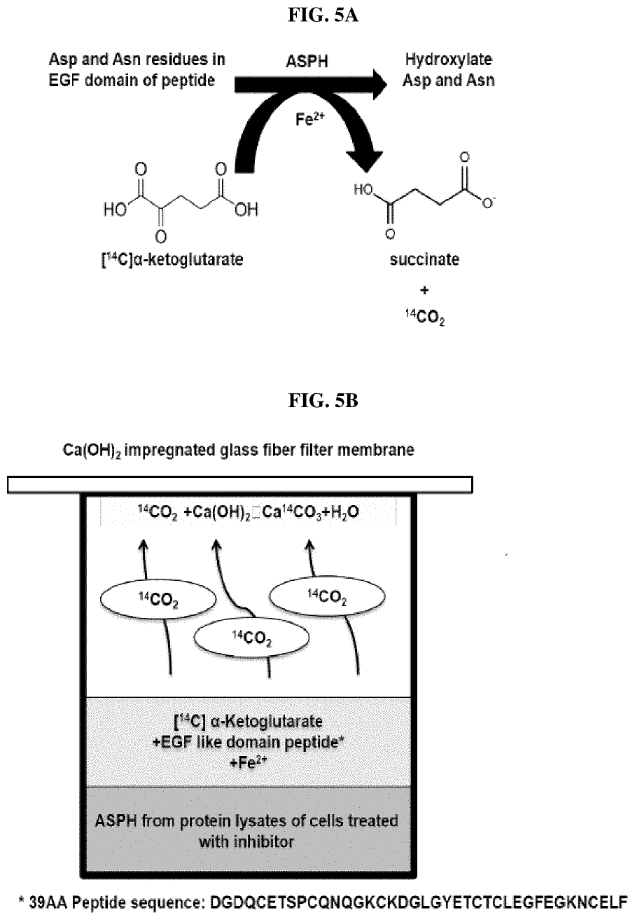

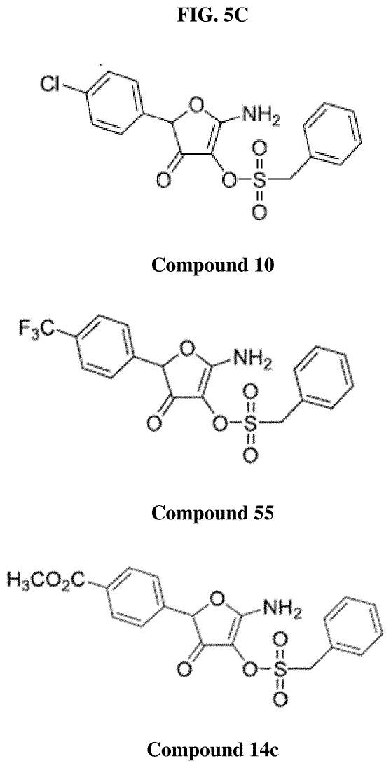

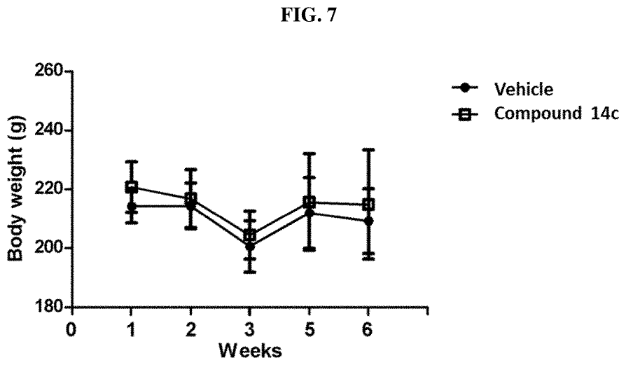

Since targeting ASPH enzymatic activity substantially inhibits CCA migration and invasion in vitro, raises the question that this enzyme may be a potential target for reducing tumor spread in vivo. To evaluate whether compound 14c suppresses CCA metastasis in vivo, we performed experiments in a rat intrahepatic syngeneic CCA model by ligating the bile duct and inoculating rat BDE-neu-CL24 cells into the liver. We first determined that compound 14c inhibited BDE-neu-CL24 cells in vitro (FIG. 4A). The intrahepatic rat model was established and the rats challenged with a control capsule or a capsule containing compound 14c (10 mg/kg, FIG. 4B) orally and daily for 18 days, and animals were sacrificed on day 21 (FIG. 4C). The body weights were measured as a gross indicator of drug toxicity and were not affected compared to vehicle control (FIG. 7). The metastatic spread to the lung surface was determined (FIG. 4D). The ASPH compound 14c substantially decreased CCA metastatic spread to the lung (FIG. 4E; p=0.041). Immunohistochemistry analysis revealed that compound 14c significantly down-regulated MMP2 and MMP14 protein expression in primary intrahepatic tumor (FIG. 4F), further supporting the hypothesis that ASPH may modulate CCA metastasis through regulating MMP expression. Collectively, these results suggest ASPH is a key component in CCA metastasis and may be a potential therapeutic target.

### Targeting Aspartate Beta-Hydroxylase Suppresses Tumors

There have been attempts to develop therapeutics that target CCA cell development, growth, and progression due to the high death rate and lack of effective therapies for CCA.

Recent whole genomic sequence information has revealed several unexplored mutations, such as IDH1 and FGFR-fusion protein and the formation of, which may be potential therapeutic targets for certain subpopulations of patients with CCA. However, the outcome of FGFR inhibitor clinical trials have not demonstrated a potential therapeutic effect since more than 80% of patients have suboptimal responses to the therapy. These findings have been attributed to other point mutations in the FGFR kinase activity. Moreover, the IDH1 inhibitor was also found not to be effective due to activation of the SRC signaling pathway. Thus, there is a need to develop effective therapeutics for IDH1 mutation and FGFR fusion protein may have potential for certain subpopulations of CCA CCA patients (IDH1 mutation in 20% and FGFR fusion protein in 16% 2-4), there are still 80% of patients in need of effective treatments. .

ASPH is highly expressed in CCA tumors and not detectable in the adjacent normal bile ducts. Several studies have suggested that targeting ASPH will suppress CCA progression by modulating cell growth, apoptosis, and cell migration. A study evaluated the potential of an ASPH specific inhibitor in repressing CCA growth in vivo by using a second generation ASPH inhibitor 9. Based on these findings, we explored a rat CCA metastatic model using the second generation ASPH inhibitor. Surprisingly, the second generation ASPH inhibitor (25 mg/kg, daily) did not achieve satisfactory anti-tumor effects on metastatic spread to the lung and primary tumor growth in the rat model. Therefore, we repeated the experiments in the context of a third generation ASPH inhibitor which has demonstrated increased potency in suppressing ASPH enzymatic activity and is more water soluble. In comparison with the previous generations of ASPH inhibitors, we found that this third generation ASPH inhibitor reduced RBE CCA cell migration by approximately 70%. This finding is significant when compared results to the first generation ASPH inhibitor where it produced only a 34% reduction. More important, the third generation ASPH inhibitor (˜10 mg/kg, daily) suppressed lung metastasis from the primary CCA tumor grown in the liver. The structures of 1st, 2nd and 3rd generation SMIs are shown in Supplemental FIG. 3. The current study further strengthens the potential of ASPH as a target for treating CCA tumors and provides a “proof-of-concept” that an oral formulation may produce significant inhibition of CCA metastatic spread.

ASPH was previously identified as a driver in CCA progression by modulation of the Notch signaling cascade. Knockdown of ASPH was found to substantially inhibit CCA migration in vitro. 9 To further clarify how ASPH modulates CCA progression, we investigated another protein family, namely the MMPs, which has been demonstrated to be highly involved in tumor metastasis33. Previously, it was observed that MMP7 and 9 may be potential biomarkers for CCA progression 34, 35 and high expression of MMP9 was associated with poor prognosis 36. Since the 5 year survival of CCA patients with metastasis is only about 2%, the linkage of MMPs to tumor cell migration may contribute to this poor prognosis, suggesting that ASPH-mediated CCA migration might not be regulated by the ASPH/Notch signaling cascade. In the current study, we provide evidence that ASPH may modulate metastatic spread in the intrahepatic tumor by altering the expression of MMPs. The immunohistochemistry staining results suggest a positive correlation between ASPH expression and upregulation of the MMPs in vivo in the rat model. Other mechanisms undoubtedly play a role Nevertheless, the MMPs inhibitor was not able to completely mask the effect of ASPH knockdown on migration. Another mechanism must be involved in ASPH-mediated metastasis spread. In this context, ASPH is an enzyme that functions through hydroxylation aspartic acid and asparagine residues located in epidermal growth factor (EGF)-like domains of various signaling molecules, such as latent transforming growth factor beta binding proteins (LTBPs). As the function of LTBPs is to orchestrate the availability of TGFβ1 which is a critical factor involved in the epithelial to mesenchymal transition in metastatic tumor tissue, ASPH modulates CCA metastatic spread through regulation of the TGFβ1 signaling cascade.

The current study provides evidence that targeting ASPH with a specific and potent third generation enzymatic inhibitor, e.g., prepared in the form of a capsule, significantly reduces metastatic spread to the lung in vivo, indicating that ASPH is a useful molecular target for treatment of this serious liver disease.

### Example 2: General Chemical Procedures

All parts are by weight (e.g., % w/w), and temperatures are in degrees centigrade (° C.), unless otherwise indicated.

Melting points were determined with a Hoover melting point apparatus and are uncorrected. Infrared (IR) spectra for the compounds were recorded in KBr discs on a Mattson Satellite FTIR in cm⁻¹. ¹H and ¹³C spectra were recorded in DMSO-d₆ on a Bruker Avance III DPX 300 MHz instrument. ¹⁹F spectra were recorded in DMSO d on a Bruker Avance III 600 (564.6 mHz). Chemical shifts were expressed in parts per million (δ) with tetramethylsilane as internal standard. Mass spectrometry was performed on a Thermo Scientific LTQ-FT at the University of Cincinnati Mass Spectrometry facility. The purity of the compounds was monitored by HPLC using a Waters 2695 separation module and a 2487 dual λ absorbance detector with a NovaPak C18 4 μm 3.9×150 mm column. The mobile phases consisted of acetonitrile/H₂O using a 30 minute gradient. All compounds were ≥95%. Microanalysis was performed by Atlantic Microlab Inc., and all compounds were found to be 0.4%. All reagents were from Sigma-Aldrich. Log S, Log P, Log BBB, human intestinal absorption, p-glycoprotein category, CYP 2C9 pKi, hERG pIC50, CYP 2D6 affinity category, oral CNS score, IV CNS score, MW, flexibility, and total polar surface area were calculated using StarDrop 5.1.1 release Build 178.

Scheme 1 illustrates the synthetic reactions used to summarize these reactions. Table 3 is a non-limiting list of aryl functional groups that can be incorporated as “Ar¹” or “Ar²” from Formulae Ia, Ib, and IIa. Table 4 illustrates the structures, names, and numbers of a variety of key compounds disclosed in in this application.

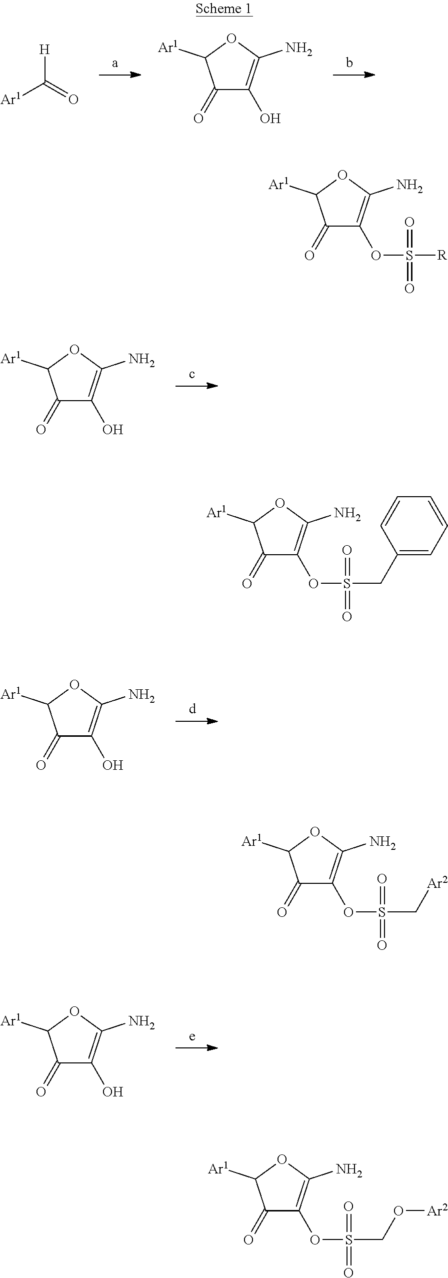

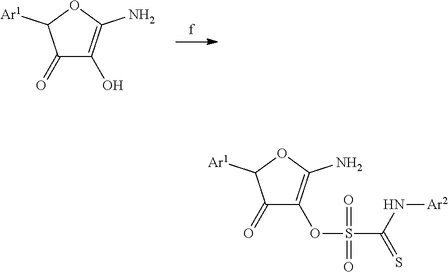

Scheme 1 above shows the synthetic strategies for compounds of Formula Ia. Reactions a-f are as follows: (a) KCN, glyoxal, Na₂CO₃, H₂O; (b) ClSO₂R, TEA, THF; (c) ClSO₂CH₂Ph, TEA, THF; (d) ClSO₂CH₂Ar², TEA, THF; (e) ClCO₂Ar², TEA, THF; (f) Ar²NCS, Na₂CO₃, H₂O.

In the reaction schemes described herein, multiple stereoisomers may be produced. When no particular stereoisomer is indicated, it is understood to mean all possible stereoisomers that could be produced from the reaction. A person of ordinary skill in the art will recognize that the reactions can be optimized to give one isomer preferentially, or new schemes may be devised to produce a single isomer. If mixtures are produced, techniques such as preparative thin layer chromatography, preparative HPLC, preparative chiral HPLC, or preparative SFC may be used to separate the isomers.

TABLE 3
A Non-Limiting List of Aryl Functional Groups “Ar¹” or “Ar²”

| Functional Group | |
|---|---|
| Structure | Ar¹ or Ar² |
| 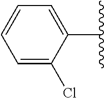 [SMILES: *c1ccccc1Cl] | 2-chlorophenyl |
| 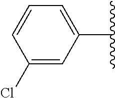 [SMILES: *c1cccc(Cl)c1] | 3-chlorophenyl |
| 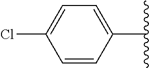 [SMILES: *c1ccc(Cl)cc1] | 4-chlorophenyl |
| 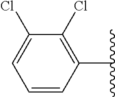 [SMILES: *c1cccc(Cl)c1Cl] | 2,3-dichlorophenyl |
| 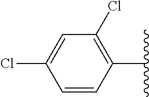 [SMILES: *c1ccc(Cl)cc1Cl] | 2,4-dichlorophenyl |
| 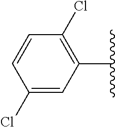 [SMILES: *c1cc(Cl)ccc1Cl] | 2,5-dichlorophenyl |
| 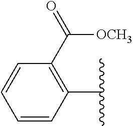 [SMILES: *c1ccccc1C(=O)OC] | 2-carboxymethylphenyl |
| 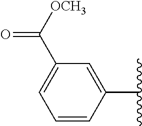 [SMILES: *c1cccc(C(=O)OC)c1] | 3-carboxymethylphenyl |
| 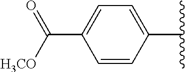 [SMILES: *c1ccc(C(=O)OC)cc1] | 4-carboxymethylphenyl |
| 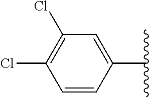 [SMILES: *c1ccc(Cl)c(Cl)c1] | 3,4-dichlorophenyl |
| 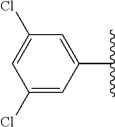 [SMILES: *c1cc(Cl)cc(Cl)c1] | 3,5-dichlorophenyl |
| 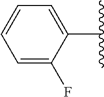 [SMILES: *c1ccccc1F] | 2-fluorophenyl |
| 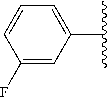 [SMILES: *c1cccc(F)c1] | 3-fluorophenyl |
| 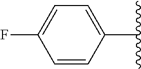 [SMILES: *c1ccc(F)cc1] | 4-fluorophenyl |
| 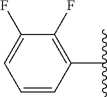 [SMILES: *c1cccc(F)c1F] | 2,3-difluorophenyl |
| 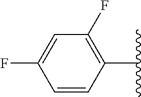 [SMILES: *c1ccc(F)cc1F] | 2,4-difluorophenyl |
| 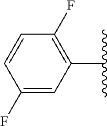 [SMILES: *c1cc(F)ccc1F] | 2,5-difluorophenyl |
| 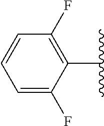 [SMILES: *c1c(F)cccc1F] | 2,6-difluorophenyl |
| 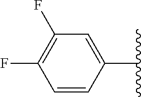 [SMILES: *c1ccc(F)c(F)c1] | 3,4-difluorophenyl |
| 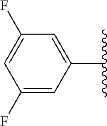 [SMILES: *c1cc(F)cc(F)c1] | 3,5-difluorophenyl |
| 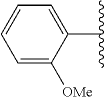 [SMILES: *c1ccccc1OC] | 2-methoxyphenyl |
| 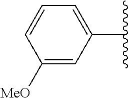 [SMILES: *c1cccc(OC)c1] | 3-methoxyphenyl |
| 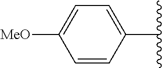 [SMILES: *c1ccc(OC)cc1] | 4-methoxyphenyl |
| 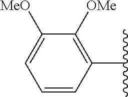 [SMILES: *c1cccc(OC)c1OC] | 2,3-dimethoxyphenyl |
| 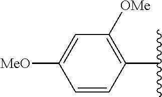 [SMILES: *c1ccc(OC)cc1OC] | 2,4-dimethoxyphenyl |
| 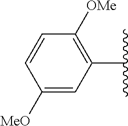 [SMILES: *c1cc(OC)ccc1OC] | 2,5-dimethoxyphenyl |
| 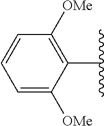 [SMILES: *c1c(OC)cccc1OC] | 2,6-dimethoxyphenyl |
| 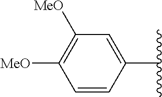 [SMILES: *c1ccc(OC)c(OC)c1] | 3,4-dimethoxyphenyl |
| 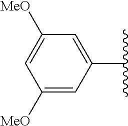 [SMILES: *c1cc(OC)cc(OC)c1] | 3,5-dimethoxyphenyl |
| 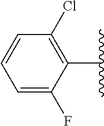 [SMILES: *c1c(F)cccc1Cl] | 2-chloro-6-fluorophenyl |
| 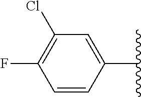 [SMILES: *c1ccc(F)c(Cl)c1] | 3-chloro-4-fluorophenyl |
| 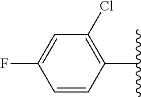 [SMILES: *c1ccc(F)cc1Cl] | 2-chloro-4-fluorophenyl |
| 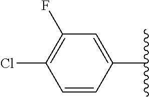 [SMILES: *c1ccc(Cl)c(F)c1] | 4-chloro-3-fluorophenyl |
| 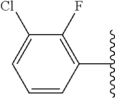 [SMILES: *c1cccc(Cl)c1F] | 3-chloro-2-fluorophenyl |
| 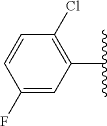 [SMILES: *c1cc(F)ccc1Cl] | 2-chloro-5-fluorophenyl |
| 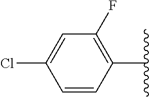 [SMILES: *c1ccc(Cl)cc1F] | 4-chloro-2-fluorophenyl |
| 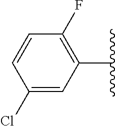 [SMILES: *c1cc(Cl)ccc1F] | 5-chloro-2-fluorophenyl |
| 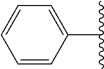 [SMILES: *c1ccccc1] | Ph |
| 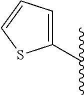 [SMILES: *c1cccs1] | 2-thiophene |
| 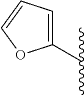 [SMILES: *c1ccco1] | 2-furan |
| 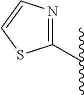 [SMILES: *c1nccs1] | 2-thiazole |
| 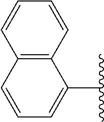 [SMILES: *c1cccc2ccccc12] | 1-naphthyl |
| 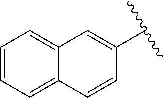 [SMILES: *c1ccc2ccccc2c1] | 2-naphthyl |
| 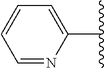 [SMILES: *c1ccccn1] | 2-pyridyl |
| 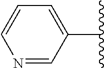 [SMILES: *c1cccnc1] | 3-pyridyl |
| 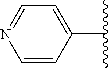 [SMILES: *c1ccncc1] | 4-pyridyl |
| 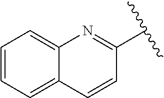 [SMILES: *c1ccc2ccccc2n1] | 2-quinolinyl |
| 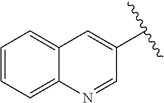 [SMILES: *c1cnc2ccccc2c1] | 3-quinolinyl |
| 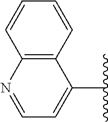 [SMILES: *c1ccnc2ccccc12] | 4-quinolinyl |
| 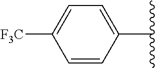 [SMILES: *c1ccc(C(F)(F)F)cc1] | 4-trifluoromethylphenyl |
| 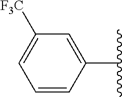 [SMILES: *c1cccc(C(F)(F)F)c1] | 3-trifluoromethylphenyl |
| 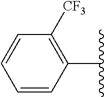 [SMILES: *c1ccccc1C(F)(F)F] | 2-trifluoromethylphenyl |
| 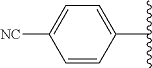 [SMILES: *c1ccc(C#N)cc1] | 4-cyanophenyl |
| 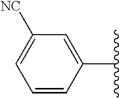 [SMILES: *c1cccc(C#N)c1] | 3-cyanophenyl |

TABLE 4
Structures, Names, and Numbers of a Variety of Key Compounds Listed in Example 2

| Compound No. | Structure | Name |
|---|---|---|
| 1 | 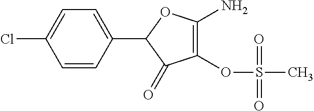 [SMILES: CS(=O)(=O)OC1=C(N)OC(c2ccc(Cl)cc2)C1=O] | 2-(4-chlorophenyl)-4- [[methylsulfonyl]oxy]-5-amino- 3(2H)-furanone |
| 2 | 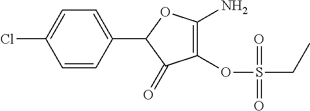 [SMILES: CCS(=O)(=O)OC1=C(N)OC(c2ccc(Cl)cc2)C1=O] | 2-(4-chlorophenyl)-4- [[ethylsulfonyl]oxy]-5-amino- 3(2H)-furanone |
| 3 | 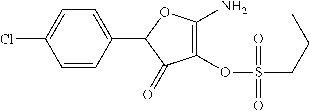 [SMILES: CCCS(=O)(=O)OC1=C(N)OC(c2ccc(Cl)cc2)C1=O] | 2-(4-chlorophenyl)-4-[[1- propylsulfonyl]oxy]-5-amino- 3(2H)-furanone |
| 4 | 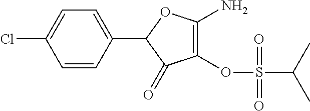 [SMILES: CC(C)S(=O)(=O)OC1=C(N)OC(c2ccc(Cl)cc2)C1=O] | 2-(4-chlorophenyl)-4-[[2- propylsulfonyl]oxy]-5-amino- 3(2H)-furanone |
| 5 | 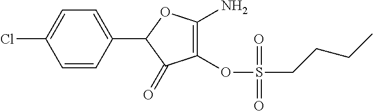 [SMILES: CCCCS(=O)(=O)OC1=C(N)OC(c2ccc(Cl)cc2)C1=O] | 2-(4-chlorophenyl)-4-[[1- butylsulfonyl]oxy]-5-amino- 3(2H)-furanone |
| 6 | 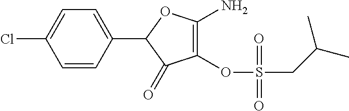 [SMILES: CC(C)CS(=O)(=O)OC1=C(N)OC(c2ccc(Cl)cc2)C1=O] | 2-(4-chlorophenyl)-4-[[1- propyl-2-methyl-sulfonyl]oxy]- 5-amino-3(2H)-furanone |
| 7 | 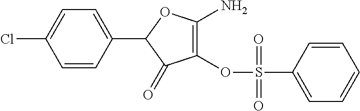 [SMILES: NC1=C(OS(=O)(=O)c2ccccc2)C(=O)C(c2ccc(Cl)cc2)O1] | 2-(4-chlorophenyl)-4- [[phenylsulfonyl]oxy]-5-amino- 3(2H)-furanone |
| 8 | 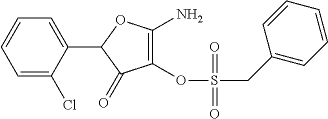 [SMILES: NC1=C(OS(=O)(=O)Cc2ccccc2)C(=O)C(c2ccccc2Cl)O1] | 2-(2-chlorophenyl)-4- [[phenylmethylsulfonyl]oxy]-5- amino-3(2H)-furanone |
| 9 | 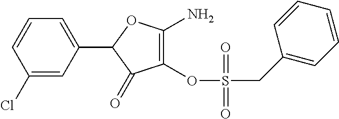 [SMILES: NC1=C(OS(=O)(=O)Cc2ccccc2)C(=O)C(c2cccc(Cl)c2)O1] | 2-(3-chlorophenyl)-4- [[phenylmethylsulfonyl]oxy]-5- amino-3(2H)-furanone |
| 10 | 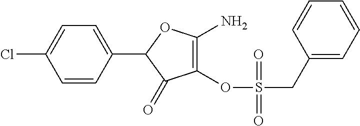 [SMILES: NC1=C(OS(=O)(=O)Cc2ccccc2)C(=O)C(c2ccc(Cl)cc2)O1] | 2-(4-chlorophenyl)-4- [[phenylmethylsulfonyl]oxy]-5- amino-3(2H)-furanone |
| 11 | 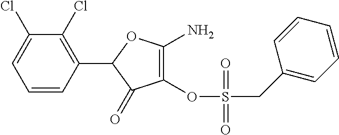 [SMILES: NC1=C(OS(=O)(=O)Cc2ccccc2)C(=O)C(c2cccc(Cl)c2Cl)O1] | 2-(2,3-dichlorophenyl)-4- [[phenylmethylsulfonyl]oxy]-5- amino-3(2H)-furanone |
| 12 | 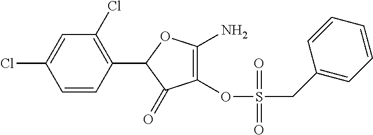 [SMILES: NC1=C(OS(=O)(=O)Cc2ccccc2)C(=O)C(c2ccc(Cl)cc2Cl)O1] | 2-(2,4-dichlorophenyl)-4- [[phenylmethylsulfonyl]oxy]-5- amino-3(2H)-furanone |
| 313 | 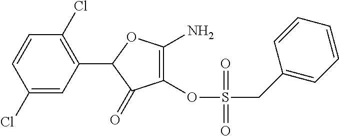 [SMILES: NC1=C(OS(=O)(=O)Cc2ccccc2)C(=O)C(c2cc(Cl)ccc2Cl)O1] | 2-(2,5-dichlorophenyl)-4- [[phenylmethylsulfonyl]oxy]-5- amino-3(2H)-furanone |
| 14a | 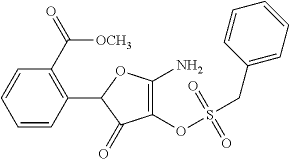 [SMILES: COC(=O)c1ccccc1C1OC(N)=C(OS(=O)(=O)Cc2ccccc2)C1=O] | 2-(2-carboxymethylphenyl)-4- [[phenylmethylsulfonyl]oxy]-5- amino-3(2H)-furanone |
| 14b | 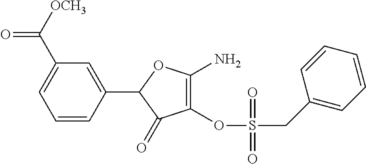 [SMILES: COC(=O)c1cccc(C2OC(N)=C(OS(=O)(=O)Cc3ccccc3)C2=O)c1] | 2-(3-carboxymethylphenyl)-4- [[phenylmethylsulfonyl]oxy]-5- amino-3(2H)-furanone |
| 14c | 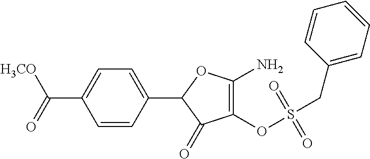 [SMILES: COC(=O)c1ccc(C2OC(N)=C(OS(=O)(=O)Cc3ccccc3)C2=O)cc1] | 2-(4-carboxymethylphenyl)-4- [[phenylmethylsulfonyl]oxy]-5- amino-3(2H)-furanone |
| 15 | 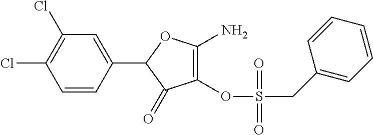 [SMILES: NC1=C(OS(=O)(=O)Cc2ccccc2)C(=O)C(c2ccc(Cl)c(Cl)c2)O1] | 2-(3,4-dichlorophenyl)-4- [[phenylmethylsulfonyl]oxy]-5- amino-3(2H)-furanone |
| 17 | 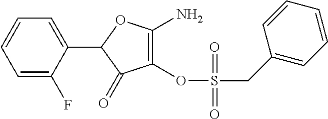 [SMILES: NC1=C(OS(=O)(=O)Cc2ccccc2)C(=O)C(c2ccccc2F)O1] | 2-(2-fluorophenyl)-4- [[phenylmethylsulfonyl]oxy]-5- amino-3(2H)-furanone |
| 18 | 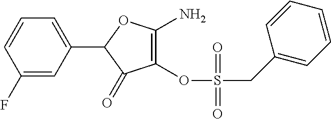 [SMILES: NC1=C(OS(=O)(=O)Cc2ccccc2)C(=O)C(c2cccc(F)c2)O1] | 2-(3-fluorophenyl)-4- [[phenylmethylsulfonyl]oxy]-5- amino-3(2H)-furanone |
| 19 | 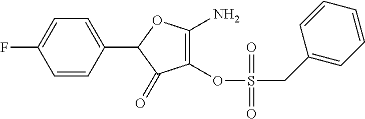 [SMILES: NC1=C(OS(=O)(=O)Cc2ccccc2)C(=O)C(c2ccc(F)cc2)O1] | 2-(4-fluorophenyl)-4- [[phenylmethylsulfonyl]oxy]-5- amino-3(2H)-furanone |
| 20 | 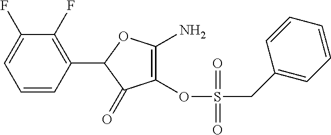 [SMILES: NC1=C(OS(=O)(=O)Cc2ccccc2)C(=O)C(c2cccc(F)c2F)O1] | 2-(2,3-difluorophenyl)-4- [[phenylmethylsulfonyl]oxy]-5- amino-3(2H)-furanone |
| 21 | 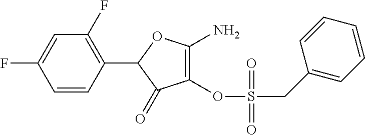 [SMILES: NC1=C(OS(=O)(=O)Cc2ccccc2)C(=O)C(c2ccc(F)cc2F)O1] | 2-(2,4-difluorophenyl)-4- [[phenylmethylsulfonyl]oxy]-5- amino-3(2H)-furanone |
| 22 | 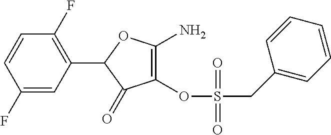 [SMILES: NC1=C(OS(=O)(=O)Cc2ccccc2)C(=O)C(c2cc(F)ccc2F)O1] | 2-(2,5-difluorophenyl)-4- [[phenylmethylsulfonyl]oxy]-5- amino-3(2H)-furanone |
| 23 | 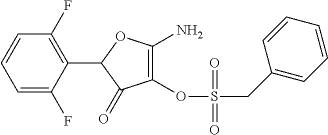 [SMILES: NC1=C(OS(=O)(=O)Cc2ccccc2)C(=O)C(c2c(F)cccc2F)O1] | 2-(2,6-difluorophenyl)-4- [[phenylmethylsulfonyl]oxy]-5- amino-3(2H)-furanone |
| 24 | 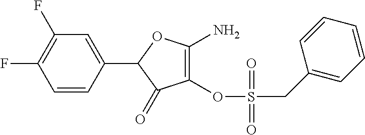 [SMILES: NC1=C(OS(=O)(=O)Cc2ccccc2)C(=O)C(c2ccc(F)c(F)c2)O1] | 2-(3,4-difluorophenyl)-4- [[phenylmethylsulfonyl]oxy]-5- amino-3(2H)-furanone |
| 25 | 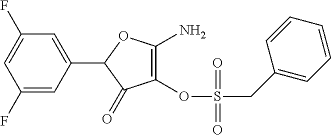 [SMILES: NC1=C(OS(=O)(=O)Cc2ccccc2)C(=O)C(c2cc(F)cc(F)c2)O1] | 2-(3,5-difluorophenyl)-4- [[phenylmethylsulfonyl]oxy]-5- amino-3(2H)-furanone |
| 26 | 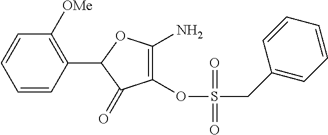 [SMILES: COc1ccccc1C1OC(N)=C(OS(=O)(=O)Cc2ccccc2)C1=O] | 2-(2-methoxyphenyl)-4- [[phenylmethylsulfonyl]oxy]-5- amino-3(2H)-furanone |
| 27 | 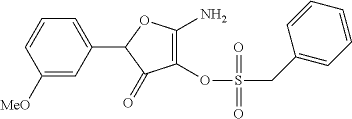 [SMILES: COc1cccc(C2OC(N)=C(OS(=O)(=O)Cc3ccccc3)C2=O)c1] | 2-(3-methoxyphenyl)-4- [[phenylmethylsulfonyl]oxy]-5- amino-3(2H)-furanone |
| 28 | 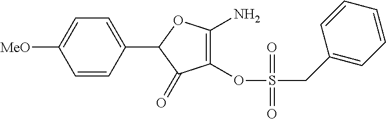 [SMILES: COc1ccc(C2OC(N)=C(OS(=O)(=O)Cc3ccccc3)C2=O)cc1] | 2-(4-methoxyphenyl)-4- [[phenylmethylsulfonyl]oxy]-5- amino-3(2H)-furanone |
| 29 | 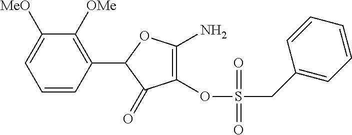 [SMILES: COc1cccc(C2OC(N)=C(OS(=O)(=O)Cc3ccccc3)C2=O)c1OC] | 2-(2,3-dimethoxyphenyl)-4- [[phenylmethylsulfonyl]oxy]-5- amino-3(2H)-furanone |
| 30 | 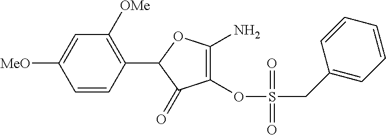 [SMILES: COc1ccc(C2OC(N)=C(OS(=O)(=O)Cc3ccccc3)C2=O)c(OC)c1] | 2-(2,4-dimethoxyphenyl)-4- [[phenylmethylsulfonyl]oxy]-5- amino-3(2H)-furanone |
| 31 | 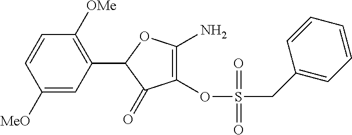 [SMILES: COc1ccc(OC)c(C2OC(N)=C(OS(=O)(=O)Cc3ccccc3)C2=O)c1] | 2-(2,5-dimethoxyphenyl)-4- [[phenylmethylsulfonyl]oxy]-5- amino-3(2H)-furanone |
| 32 | 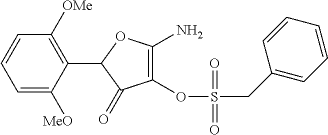 [SMILES: COc1cccc(OC)c1C1OC(N)=C(OS(=O)(=O)Cc2ccccc2)C1=O] | 2-(2,6-dimethoxyphenyl)-4- [[phenylmethylsulfonyl]oxy]-5- amino-3(2H)-furanone |
| 33 | 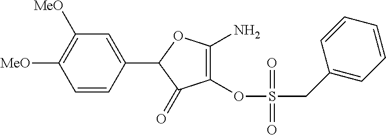 [SMILES: COc1ccc(C2OC(N)=C(OS(=O)(=O)Cc3ccccc3)C2=O)cc1OC] | 2-(3,4- dimethoxyphenyl)-4- [[phenylmethylsulfonyl] oxy]-5-amino-3(2H)- furanone |
| 34 | 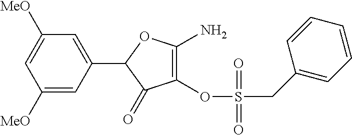 [SMILES: COc1cc(OC)cc(C2OC(N)=C(OS(=O)(=O)Cc3ccccc3)C2=O)c1] | 2-(3,5-dimethoxyphenyl)-4- [[phenylmethylsulfonyl]oxy]-5- amino-3(2H)-furanone |
| 35 | 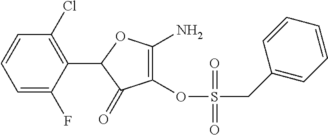 [SMILES: NC1=C(OS(=O)(=O)Cc2ccccc2)C(=O)C(c2c(F)cccc2Cl)O1] | 2-(2-chloro-6-fluorophenyl)-4- [[phenylmethylsulfonyl]oxy]-5- amino-3(2H)-furanone |
| 36 | 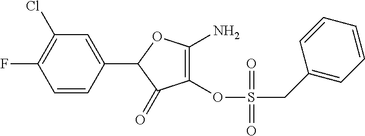 [SMILES: NC1=C(OS(=O)(=O)Cc2ccccc2)C(=O)C(c2ccc(F)c(Cl)c2)O1] | 2-(3-chloro-4-fluorophenyl)-4- [[phenylmethylsulfonyl]oxy]-5- amino-3(2H)-furanone |
| 37 | 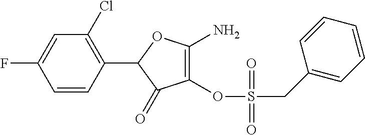 [SMILES: NC1=C(OS(=O)(=O)Cc2ccccc2)C(=O)C(c2ccc(F)cc2Cl)O1] | 2-(2-chloro-4-fluorophenyl)-4- [[phenylmethylsulfonyl]oxy]-5- amino-3(2H)-furanone |
| 38 | 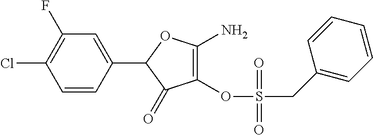 [SMILES: NC1=C(OS(=O)(=O)Cc2ccccc2)C(=O)C(c2ccc(Cl)c(F)c2)O1] | 2-(4-chloro-3-fluorophenyl)-4- [[phenylmethylsulfonyl]oxy]-5- amino-3(2H)-furanone |
| 39 | 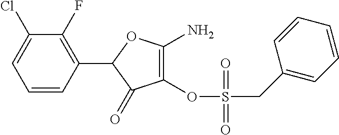 [SMILES: NC1=C(OS(=O)(=O)Cc2ccccc2)C(=O)C(c2cccc(Cl)c2F)O1] | 2-(3-chloro-2-fluorophenyl)-4- [[phenylmethylsulfonyl]oxy]-5- amino-3(2H)-furanone |
| 40 | 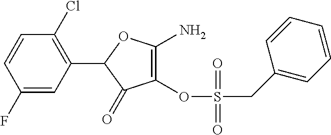 [SMILES: NC1=C(OS(=O)(=O)Cc2ccccc2)C(=O)C(c2cc(F)ccc2Cl)O1] | 2-(2-chloro-5-fluorophenyl)-4- [[phenylmethylsulfonyl]oxy]-5- amino-3(2H)-furanone |
| 41 | 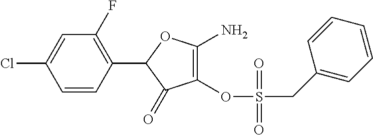 [SMILES: NC1=C(OS(=O)(=O)Cc2ccccc2)C(=O)C(c2ccc(Cl)cc2F)O1] | 2-(4-chloro-2-fluorophenyl)-4- [[phenylmethylsulfonyl]oxy]-5- amino-3(2H)-furanone |
| 42 | 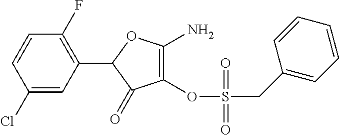 [SMILES: NC1=C(OS(=O)(=O)Cc2ccccc2)C(=O)C(c2cc(Cl)ccc2F)O1] | 2-(3-chloro-5-fluorophenyl)-4- [[phenylmethylsulfonyl]oxy]-5- amino-3(2H)-furanone |
| 43 | 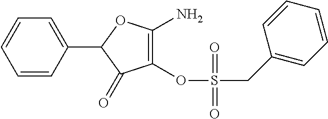 [SMILES: NC1=C(OS(=O)(=O)Cc2ccccc2)C(=O)C(c2ccccc2)O1] | 2-(phenyl)-4- [[phenylmethylsulfonyl]oxy]-5- amino-3(2H)-furanone |
| 44 | 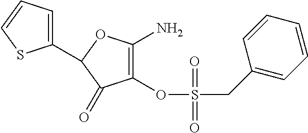 [SMILES: NC1=C(OS(=O)(=O)Cc2ccccc2)C(=O)C(c2cccs2)O1] | 2-(2-thiophene)-4- [[phenylmethylsulfonyl]oxy]-5- amino-3(2H)-furanone |
| 45 | 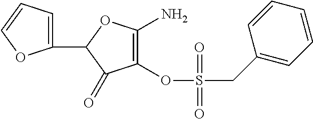 [SMILES: NC1=C(OS(=O)(=O)Cc2ccccc2)C(=O)C(c2ccco2)O1] | 2-(2-furanyl)-4- [[phenylmethylsulfonyl]oxy]-5- amino-3(2H)-furanone |
| 46 | 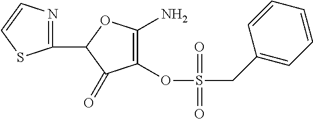 [SMILES: NC1=C(OS(=O)(=O)Cc2ccccc2)C(=O)C(c2nccs2)O1] | 2-(2-thiazolyl)-4- [[phenylmethylsulfonyl]oxy]-5- amino-3(2H)-furanone |
| 47 | 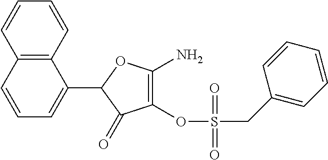 [SMILES: NC1=C(OS(=O)(=O)Cc2ccccc2)C(=O)C(c2cccc3ccccc23)O1] | 2-(1-naphthyl)-4- [[phenylmethylsulfonyl]oxy]-5- amino-3(2H)-furanone |
| 48 | 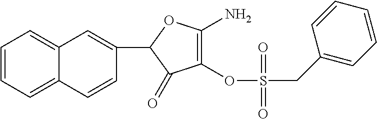 [SMILES: NC1=C(OS(=O)(=O)Cc2ccccc2)C(=O)C(c2ccc3ccccc3c2)O1] | 2-(2-naphthyl)-4- [[phenylmethylsulfonyl]oxy]-5- amino-3(2H)-furanone |
| 49 | 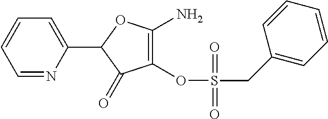 [SMILES: NC1=C(OS(=O)(=O)Cc2ccccc2)C(=O)C(c2ccccn2)O1] | 2-(2-pyridyl)-4- [[phenylmethylsulfonyl]oxy]-5- amino-3(2H)-furanone |
| 50 | 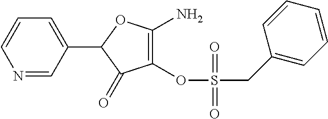 [SMILES: NC1=C(OS(=O)(=O)Cc2ccccc2)C(=O)C(c2cccnc2)O1] | 2-(3-pyridyl)-4- [[phenylmethylsulfonyl]oxy]-5- amino-3(2H)-furanone |
| 51 | 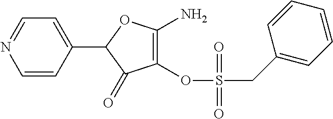 [SMILES: NC1=C(OS(=O)(=O)Cc2ccccc2)C(=O)C(c2ccncc2)O1] | 2-(4-pyridyl)-4- [[phenylmethylsulfonyl]oxy]-5- amino-3(2H)-furanone |
| 52 | 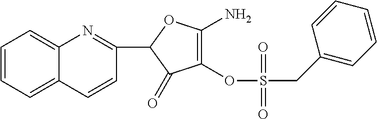 [SMILES: NC1=C(OS(=O)(=O)Cc2ccccc2)C(=O)C(c2ccc3ccccc3n2)O1] | 2-(2-quinolinyl)-4- [[phenylmethylsulfonyl]oxy]-5- amino-3(2H)-furanone |
| 53 | 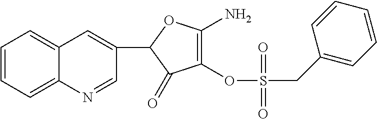 [SMILES: NC1=C(OS(=O)(=O)Cc2ccccc2)C(=O)C(c2cnc3ccccc3c2)O1] | 2-(3-quinolinyl)-4- [[phenylmethylsulfonyl]oxy]-5- amino-3(2H)-furanone |
| 54 | 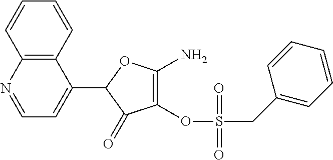 [SMILES: NC1=C(OS(=O)(=O)Cc2ccccc2)C(=O)C(c2ccnc3ccccc23)O1] | 2-(4-quinolinyl)-4- [[phenylmethylsulfonyl]oxy]-5- amino-3(2H)-furanone |
| 55 | 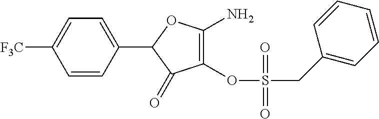 [SMILES: NC1=C(OS(=O)(=O)Cc2ccccc2)C(=O)C(c2ccc(C(F)(F)F)cc2)O1] | 2-(4-trifluoromethylphenyl)-4- [[phenylmethylsulfonyl]oxy]-5- amino-3(2H)-furanone |
| 56 | 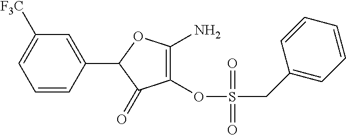 [SMILES: NC1=C(OS(=O)(=O)Cc2ccccc2)C(=O)C(c2cccc(C(F)(F)F)c2)O1] | 2-(3-trifluoromethylphenyl)-4- [[phenylmethylsulfonyl]oxy]-5- amino-3(2H)-furanone |
| 57 | 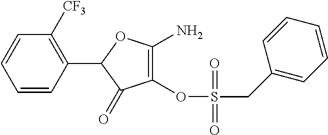 [SMILES: NC1=C(OS(=O)(=O)Cc2ccccc2)C(=O)C(c2ccccc2C(F)(F)F)O1] | 2-(2-trifluoromethylphenyl)-4- [[phenylmethylsulfonyl]oxy]-5- amino-3(2H)-furanone |
| 58 | 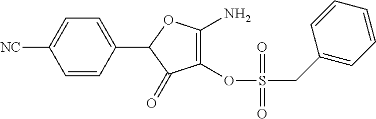 [SMILES: N#Cc1ccc(C2OC(N)=C(OS(=O)(=O)Cc3ccccc3)C2=O)cc1] | 2-(4-nitrilephenyl)-4- [[phenylmethylsulfonyl]oxy]-5- amino-3(2H)-furanone |
| 59 | 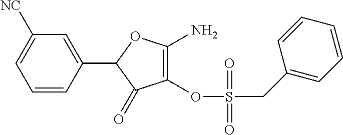 [SMILES: N#Cc1cccc(C2OC(N)=C(OS(=O)(=O)Cc3ccccc3)C2=O)c1] | 2-(3-nitrilephenyl)-4- [[phenylmethylsulfonyl]oxy]-5- amino-3(2H)-furanone |
| 60 | 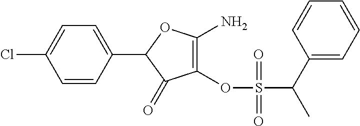 [SMILES: CC(c1ccccc1)S(=O)(=O)OC1=C(N)OC(c2ccc(Cl)cc2)C1=O] | 2-(4-chlorophenyl)-4-[[1- phenylethylsulfonyl]oxy]-5- amino-3(2H)-furanone |
| 61 | 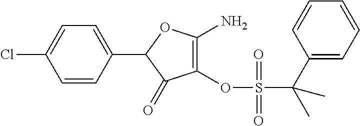 [SMILES: CC(C)(c1ccccc1)S(=O)(=O)OC1=C(N)OC(c2ccc(Cl)cc2)C1=O] | 2-(4-chlorophenyl)-4-[[1- methyl-1- phenylethylsulfonyl]oxy]-5- amino-3(2H)-furanone |
| 62 | 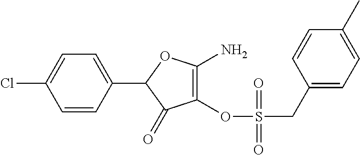 [SMILES: Cc1ccc(CS(=O)(=O)OC2=C(N)OC(c3ccc(Cl)cc3)C2=O)cc1] | 2-(4-chlorophenyl)-4-[[4- methylphenylmethylsulfonyl] oxy]-5-amino-3(2H)-furanone |
| 63 | 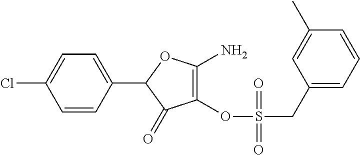 [SMILES: Cc1cccc(CS(=O)(=O)OC2=C(N)OC(c3ccc(Cl)cc3)C2=O)c1] | 2-(4-chlorophenyl)-4-[[3- methylphenylmethylsulfonyl] oxy]-5-amino-3(2H)-furanone |
| 64 | 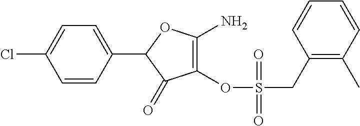 [SMILES: Cc1ccccc1CS(=O)(=O)OC1=C(N)OC(c2ccc(Cl)cc2)C1=O] | 2-(4-chlorophenyl)-4-[[2- methylphenylmethylsulfonyl] oxy]-5-amino-3(2H)-furanone |
| 65 | 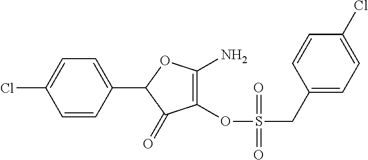 [SMILES: NC1=C(OS(=O)(=O)Cc2ccc(Cl)cc2)C(=O)C(c2ccc(Cl)cc2)O1] | 2-(4-chlorophenyl)-4-[[4- chlorophenylmethylsulfonyl] oxy]-5-amino-3(2H)-furanone |
| 66 | 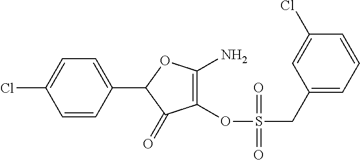 [SMILES: NC1=C(OS(=O)(=O)Cc2cccc(Cl)c2)C(=O)C(c2ccc(Cl)cc2)O1] | 2-(4-chlorophenyl)-4-[[3- chlorophenylmethylsulfonyl] oxy]-5-amino-3(2H)-furanone |
| 67 | 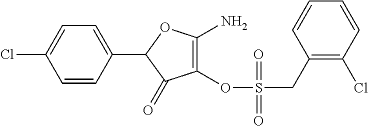 [SMILES: NC1=C(OS(=O)(=O)Cc2ccccc2Cl)C(=O)C(c2ccc(Cl)cc2)O1] | 2-(4-chlorophenyl)-4-[[2- chlorophenylmethylsulfonyl] oxy]-5-amino-3(2H)-furanone |
| 68 | 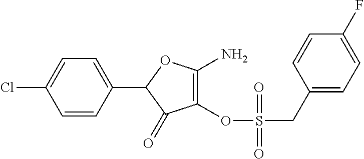 [SMILES: NC1=C(OS(=O)(=O)Cc2ccc(F)cc2)C(=O)C(c2ccc(Cl)cc2)O1] | 2-(4-chlorophenyl)-4-[[4- fluorophenylmethylsulfonyl] oxy]-5-amino-3(2H)-furanone |
| 69 | 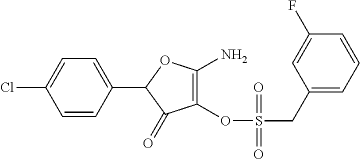 [SMILES: NC1=C(OS(=O)(=O)Cc2cccc(F)c2)C(=O)C(c2ccc(Cl)cc2)O1] | 2-(4-chlorophenyl)-4-[[3- fluorophenylmethylsulfonyl] oxy]-5-amino-3(2H)-furanone |
| 70 | 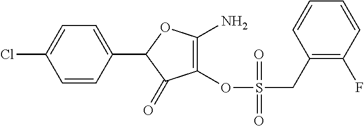 [SMILES: NC1=C(OS(=O)(=O)Cc2ccccc2F)C(=O)C(c2ccc(Cl)cc2)O1] | 2-(4-chlorophenyl)-4-[[2- fluorophenylmethylsulfonyl] oxy]-5-amino-3(2H)-furanone |
| 71 | 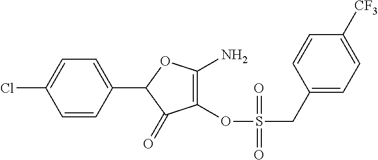 [SMILES: NC1=C(OS(=O)(=O)Cc2ccc(C(F)(F)F)cc2)C(=O)C(c2ccc(Cl)cc2)O1] | 2-(4-chlorophenyl)-4-[[4- trifluoromethylphenylmethyl- sulfonyl]oxy]-5-amino-3(2H)- furanone |
| 72 | 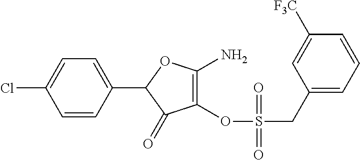 [SMILES: NC1=C(OS(=O)(=O)Cc2cccc(C(F)(F)F)c2)C(=O)C(c2ccc(Cl)cc2)O1] | 2-(4-chlorophenyl)-4-[[3- trifluoromethylphenylmethyl- sulfonyl]oxy]-5-amino-3(2H)- furanone |
| 73 | 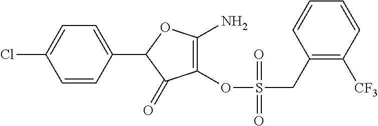 [SMILES: NC1=C(OS(=O)(=O)Cc2ccccc2C(F)(F)F)C(=O)C(c2ccc(Cl)cc2)O1] | 2-(4-chlorophenyl)-4-[[2- trifluoromethylphenylmethyl- sulfonyl]oxy]-5-amino-3(2H)- furanone |
| 74 | 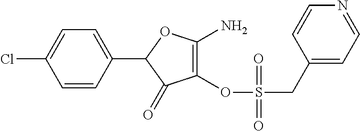 [SMILES: NC1=C(OS(=O)(=O)Cc2ccncc2)C(=O)C(c2ccc(Cl)cc2)O1] | 2-(4-chlorophenyl)-4-[[4- pyridylmethylsulfonyl]oxy]-5- amino-3(2H)-furanone |
| 75 | 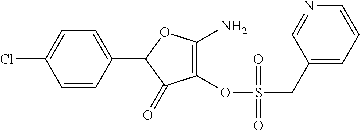 [SMILES: NC1=C(OS(=O)(=O)Cc2cccnc2)C(=O)C(c2ccc(Cl)cc2)O1] | 2-(4-chlorophenyl)-4-[[3- pyridylmethylsulfonyl]oxy]-5- amino-3(2H)-furanone |
| 76 | 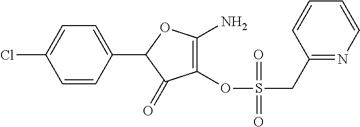 [SMILES: NC1=C(OS(=O)(=O)Cc2ccccn2)C(=O)C(c2ccc(Cl)cc2)O1] | 2-(4-chlorophenyl)-4-[[2- pyridylmethylsulfonyl]oxy]-5- amino-3(2H)-furanone |
| 77 | 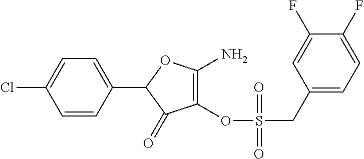 [SMILES: NC1=C(OS(=O)(=O)Cc2ccc(F)c(F)c2)C(=O)C(c2ccc(Cl)cc2)O1] | 2-(4-chlorophenyl)-4- [[3,4- difluorophenylmethyl- sulfonyl]oxy]-5-amino- 3(2H)-furanone |
| 78 | 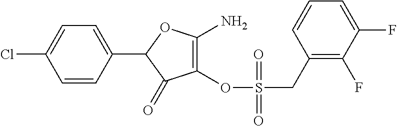 [SMILES: NC1=C(OS(=O)(=O)Cc2cccc(F)c2F)C(=O)C(c2ccc(Cl)cc2)O1] | 2-(4-chlorophenyl)-4-[[2,3- difluorophenylmethylsulfonyl] oxy]-5-amino-3(2H)-furanone |
| 79 | 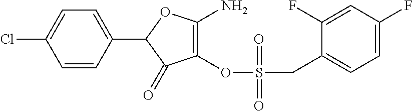 [SMILES: NC1=C(OS(=O)(=O)Cc2ccc(F)cc2F)C(=O)C(c2ccc(Cl)cc2)O1] | 2-(4-chlorophenyl)-4-[[2,4- difluorophenylmethylsulfonyl] oxy]-5-amino-3(2H)-furanone |
| 80 | 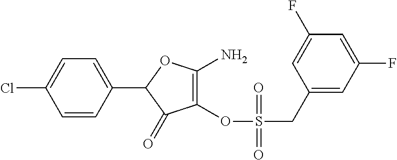 [SMILES: NC1=C(OS(=O)(=O)Cc2cc(F)cc(F)c2)C(=O)C(c2ccc(Cl)cc2)O1] | 2-(4-chlorophenyl)-4-[[3,5- difluorophenylmethylsulfonyl] oxy]-5-amino-3(2H)-furanone |
| 81 | 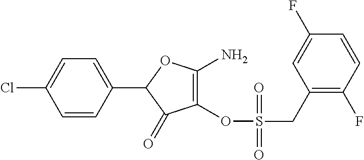 [SMILES: NC1=C(OS(=O)(=O)Cc2cc(F)ccc2F)C(=O)C(c2ccc(Cl)cc2)O1] | 2-(4-chlorophenyl)-4-[[2,5- difluorophenylmethylsulfonyl] oxy]-5-amino-3(2H)-furanone |
| 82 | 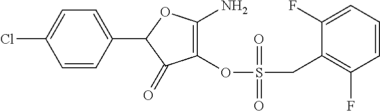 [SMILES: NC1=C(OS(=O)(=O)Cc2c(F)cccc2F)C(=O)C(c2ccc(Cl)cc2)O1] | 2-(4-chlorophenyl)-4-[[2,6- difluorophenylmethylsulfonyl] oxy]-5-amino-3(2H)-furanone |
| 83 | 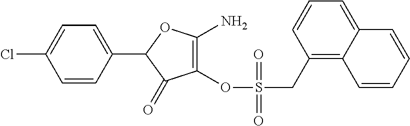 [SMILES: NC1=C(OS(=O)(=O)Cc2cccc3ccccc23)C(=O)C(c2ccc(Cl)cc2)O1] | 2-(4-chlorophenyl)-4-[[1- naphthylmethylsulfonyl]oxy]-5- amino-3(2H)-furanone |
| 84 | 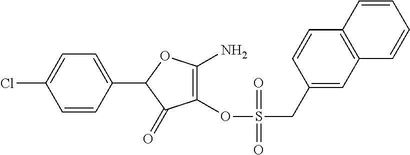 [SMILES: NC1=C(OS(=O)(=O)Cc2ccc3ccccc3c2)C(=O)C(c2ccc(Cl)cc2)O1] | 2-(4-chlorophenyl)-4-[[2- naphthylmethylsulfonyl]oxy]-5- amino-3(2H)-furanone |
| 85 | 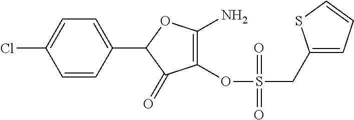 [SMILES: NC1=C(OS(=O)(=O)Cc2cccs2)C(=O)C(c2ccc(Cl)cc2)O1] | 2-(4-chlorophenyl)-4-[[2- thiophenemethylsulfonyl]oxy]- 5-amino-3(2H)-furanone |
| 86 | 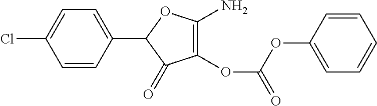 [SMILES: NC1=C(OC(=O)Oc2ccccc2)C(=O)C(c2ccc(Cl)cc2)O1] | 2-(4-chlorophenyl)-4- [[phenoxycarbonyl]oxy]-5- amino-3(2H)-furanone |
| 87 | 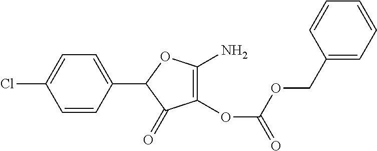 [SMILES: NC1=C(OC(=O)OCc2ccccc2)C(=O)C(c2ccc(Cl)cc2)O1] | 2-(4-chlorophenyl)-4- [[benzyloxycarbonyl]oxy]-5- amino-3(2H)-furanone |
| 88 | 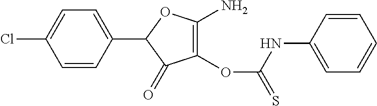 [SMILES: NC1=C(OC(=S)Nc2ccccc2)C(=O)C(c2ccc(Cl)cc2)O1] | 2-(4-chlorophenyl)-4- [[phenylaminothiocarbonyl]oxy]- 5-amino-3(2H)-furanone |
| 89 | 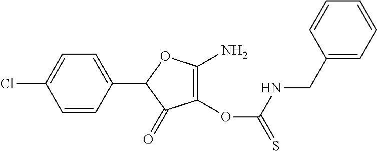 [SMILES: NC1=C(OC(=S)NCc2ccccc2)C(=O)C(c2ccc(Cl)cc2)O1] | 2-(4-chlorophenyl)-4- [[benzylaminothiocarbonyl]oxy]- 5-amino-3(2H)-furanone |

### General Procedures for Preparation of Aryltetronimides

Potassium cyanide (0.91 g) was added to sodium carbonate (1.7 g) in deionized water (30 mL) in a 3-Neck Glass Round Flask and placed in an ice bath. The system was repeatedly purged using a vacuum pump and nitrogen gas. Glyoxal (3.72 g) was then added to the system without the introduction of O₂ and the reactants were allowed to dissolve with stirring. In a stoppered tube, the appropriate arylaldehyde (7.11 mmoles) was added to 1,4-dioxane (5 mL), purged, and then added drop-wise to the system. The system was then removed from the ice bath and allowed to stir at room temperature for 1 hour. After 1 hour, acetic acid (5 mL) was added drop-wise until gas bubbles were no longer visible from the addition of acetic acid, or until the solution was at a pH of less than 6. The solution was vacuum filtered and washed with ice cold water (5 mL), methanol (5 mL) and ether (5 mL) and then was allowed to air dry. Crude material was recrystallized with methanol, collected by vacuum filtration and rinsed with diethyl ether and dried under vacuum.

TABLE 5
Table of Nucleotide and Amino Acid Sequences Supported in the Specification

| | | | | SEQ |
|---|---|---|---|---|
| Name | Description | Length | Type | ID NO |
| EGF-like domain | DGDQCETSPC QNQGKCKDGL | 39 | AA | 1 |
| peptide | GEYTCTCLE GFEGKNCELF | | | |
| EGF-like domain | CDXXXCXXK XGNGXCDXXC | 31 | AA | 2 |
| peptide consensus | NNAACXXDGX DC | | | |
| sequence | | | | |
| cDNA sequence of | cggaccgtgc aatggcccag | 2324 | DNA | 3 |
| human ASPH | cgtaagaatg ccaagagcag | | | |
| (GENBANK Accession | cggcaacagc agcagcagcg 61 | | | |
| No. S83325; codon | gctccggcag cggtagcacg | | | |
| encoding initiating | agtgcgggca gcagcagccc | | | |
| methionine is | cggggcccgg agagagacaa 121 | | | |
| underlined) | agcatggagg acacaagaat | | | |
| | gggaggaaag gcggactctc | | | |
| | gggaacttca ttcttcacgt 181 | | | |
| | ggtttatggt gattgcattg | | | |
| | ctgggcgtct ggacatctgt | | | |
| | agctgtcgtt tggtttgatc 241 | | | |
| | ttgttgacta tgaggaagtt | | | |
| | ctaggaaaac taggaatcta | | | |
| | tgatgctgat ggtgatggag 301 | | | |
| | attttgatgt ggatgatgcc | | | |
| | aaagttttat taggacttaa | | | |
| | agagagatct acttcagagc 361 | | | |
| | cagcagtccc gccagaagag | | | |
| | gctgagccac acactgagcc | | | |
| | cgaggagcag gttcctgtgg 421 | | | |
| | aggcagaacc ccagaatatc | | | |
| | gaagatgaag caaaagaaca | | | |
| | aattcagtcc cttctccatg 481 | | | |
| | aaatggtaca cgcagaacat | | | |
| | gttgagggag aagacttgca | | | |
| | acaagaagat ggacccacag 541 | | | |
| | gagaaccaca acaagaggat | | | |
| | gatgagtttc ttatggcgac | | | |
| | tgatgtagat gatagatttg 601 | | | |
| | agaccctgga acctgaagta | | | |
| | tctcatgaag aaaccgagca | | | |
| | tagttaccac gtggaagaga 661 | | | |
| | cagtttcaca agactgtaat | | | |
| | caggatatgg aagagatgat | | | |
| | gtctgagcag gaaaatccag 721 | | | |
| | attccagtga accagtagta | | | |
| | gaagatgaaa gattgcacca | | | |
| | tgatacagat gatgtaacat 781 | | | |
| | accaagtcta tgaggaacaa | | | |
| | gcagtatatg aacctctaga | | | |
| | aaatgaaggg atagaaatca 841 | | | |
| | cagaagtaac tgctccccct | | | |
| | gaggataatc ctgtagaaga | | | |
| | ttcacaggta attgtagaag 901 | | | |
| | aagtaagcat ttttcctgtg | | | |
| | gaagaacagc aggaagtacc | | | |
| | accagaaaca aatagaaaaa 961 | | | |
| | cagatgatcc agaacaaaaa | | | |
| | gcaaaagtta agaaaaagaa | | | |
| | gcctaaactt ttaaataaat 1021 | | | |
| | ttgataagac tattaaagct | | | |
| | gaacttgatg ctgcagaaaa | | | |
| | actccgtaaa aggggaaaaa 1081 | | | |
| | ttgaggaagc agtgaatgca | | | |
| | tttaaagaac tagtacgcaa | | | |
| | ataccctcag agtccacgag 1141 | | | |
| | caagatatgg gaaggcgcag | | | |
| | tgtgaggatg atttggctga | | | |
| | gaagaggaga agtaatgagg 1201 | | | |
| | tgctacgtgg agccatcgag | | | |
| | acctaccaag aggtggccag | | | |
| | cctacctgat gtccctgcag 1261 | | | |
| | acctgctgaa gctgagtttg | | | |
| | aagcgtcgct cagacaggca | | | |
| | acaatttcta ggtcatatga 1321 | | | |
| | gaggttccct gcttaccctg | | | |
| | cagagattag ttcaactatt | | | |
| | tcccaatgat acttccttaa 1381 | | | |
| | aaaatgacct tggcgtggga | | | |
| | tacctcttga taggagataa | | | |
| | tgacaatgca aagaaagttt 1441 | | | |
| | atgaagaggt gctgagtgtg | | | |
| | acacctaatg atggctttgc | | | |
| | taaagtccat tatggcttca 1501 | | | |
| | tcctgaaggc acagaacaaa | | | |
| | attgctgaga gcatcccata | | | |
| | tttaaaggaa ggaatagaat 1561 | | | |
| | ccggagatcc tggcactgat | | | |
| | gatgggagat tttatttcca | | | |
| | cctgggggat gccatgcaga 1621 | | | |
| | gggttgggaa caaagaggca | | | |
| | tataagtggt atgagcttgg | | | |
| | gcacaagaga ggacactttg 1681 | | | |
| | catctgtctg gcaacgctca | | | |
| | ctctacaatg tgaatggact | | | |
| | gaaagcacag ccttggtgga 1741 | | | |
| | ccccaaaaga aacgggctac | | | |
| | acagagttag taaagtcttt | | | |
| | agaaagaaac tggaagttaa 1801 | | | |
| | tccgagatga aggccttgca | | | |
| | gtgatggata aagccaaagg | | | |
| | tctcttcctg cctgaggatg 1861 | | | |
| | aaaacctgag ggaaaaaggg | | | |
| | gactggagcc agttcacgct | | | |
| | gtggcagcaa ggaagaagaa 1921 | | | |
| | atgaaaatgc ctgcaaagga | | | |
| | gctcctaaaa cctgtacctt | | | |
| | actagaaaag ttccccgaga 1981 | | | |
| | caacaggatg cagaagagga | | | |
| | cagatcaaat attccatcat | | | |
| | gcaccccggg actcacgtgt 2041 | | | |
| | ggccgcacac agggcccaca | | | |
| | aactgcaggc tccgaatgca | | | |
| | cctgggcttg gtgattccca 2101 | | | |
| | aggaaggctg caagattcga | | | |
| | tgtgccaacg agaccaggac | | | |
| | ctgggaggaa ggcaaggtgc 2161 | | | |
| | tcatctttga tgactccttt | | | |
| | gagcacgagg tatggcagga | | | |
| | tgcctcatct ttccggctga 2221 | | | |
| | tattcatcgt ggatgtgtgg | | | |
| | catccggaac tgacaccaca | | | |
| | gcagagacgc agccttccag 2281 | | | |
| | caatttagca tgaattcatg | | | |
| | caagcttggg aaactctgga gaga | | | |
| Amino acid sequence of | MAQRKNAKSS GNSSSSGSGS | 758 | AA | 4 |
| human ASPH (GenBank | GSTSAGSSSP GARRETKHGG | | | |
| Accession No. S83325; | HKNGRKGGLS GTSFFTWFMV 61 | | | |
| His motif is | IALLGVWTSV AVVWFDLVDY | | | |
| underlined; | EEVLGKLGIY DADGDGDFDV | | | |
| conserved sequences | DDAKVLLGLK ERSTSEPAVP 121 | | | |
| within the catalytic | PEEAEPHTEP EEQVPVEAEP | | | |
| domain are designated | QNIEDEAKEQ IQSLLHEMVH | | | |
| by bold type) | AEHVEGEDLQ QEDGPTGEPQ 181 | | | |
| | QEDDEFLMAT DVDDRFETLE | | | |
| | PEVSHEETEH SYHVEETVSQ | | | |
| | DCNQDMEEMM SEQENPDSSE 241 | | | |
| | PVVEDERLHH DTDDVTYQVY | | | |
| | EEQAVYEPLE NEGIEITEVT | | | |
| | APPEDNPVED SQVIVEEVSI 301 | | | |
| | FPVEEQQEVP PETNRKTDDP | | | |
| | EQKAKVKKKK PKLLNKFDKT | | | |
| | IKAELDAAEK LRKRGKIEEA 361 | | | |
| | VNAFKELVRK YPQSPRARYG | | | |
| | KAQCEDDLAE KRRSNEVLRG | | | |
| | AIETYQEVAS LPDVPADLLK 421 | | | |
| | LSLKRRSDRQ QFLGHMRGSL | | | |
| | LTLQRLVQLF PNDTSLKNDL | | | |
| | GVGYLLIGDN DNAKKVYEEV 481 | | | |
| | LSVTPNDGFA KVHYGFILKA | | | |
| | QNKIAESIPY LKEGIESGDP | | | |
| | GTDDGRFYFH LGDAMQRVGN 541 | | | |
| | KEAYKWYELG HKRGHFASVW | | | |
| | QRSLYNVNGL KAQPWWTPKE | | | |
| | TGYTELVKSL ERNWKLIRDE 601 | | | |
| | GLAVMDKAKG LFLPEDENLR | | | |
| | EKGDWSQFTL WQQGRRNENA | | | |
| | CKGAPKTCTL LEKFPETTGC 661 | | | |
| | RRGQIKYSIM HPGTHVWPHT | | | |
| | GPTNCRLRMH LGLVIPKEGC | | | |
| | KIRCANETRT WEEGKVLIFD 721 | | | |
| | DSFEHEVWQD ASSFRLIFIV | | | |
| | DVWHPELTPQ QRRSLPAI | | | |

### REFERENCES

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

### OTHER EMBODIMENTS

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating a cell proliferative disease comprising administering a therapeutically effective amount of an asparatyl (asparaginyl) beta-hydroxylase (ASPH) inhibitor to a subject in need thereof.

2. The method of claim 1, wherein the ASPH inhibitor has a structure of

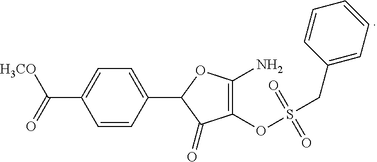 [SMILES: COC(=O)c1ccc(C2OC(N)=C(OS(=O)(=O)Cc3ccccc3)C2=O)cc1]

3. The method of claim 1, wherein the cell proliferative disease comprises a cancer.

4. The method of claim 3, wherein cells of the cancer express greater level of ASPH than normal cells.

5. The method of claim 4, wherein the ASPH inhibitor suppresses metastasis in the cancer cells.

6. The method of claim 4, wherein the cancer comprises liver cancer, pancreatic cancer, lung cancer, colon cancer, breast cancer, gastric cancer, sarcoma, renal cancer, prostate cancer, brain cancer, or leukemia.

7. The method of claim 4, wherein the liver cancer comprises hepatocellular cancer, or cholangiocarcinoma.

8. The method of claim 4, wherein the lung cancer comprises small cell lung cancer, non-small cell lung cancer, or metastatic lung cancer.

9. The method of claim 4, wherein the brain cancer comprises gliomas, meningioma, astrocytomas, or glioblastoma.

10. The method of claim 4, wherein the breast cancer comprises ductal carcinoma, triple negative breast cancer, inflammatory breast cancer, metastatic breast cancer, medullary carcinoma, tubular carcinoma, or mucinous carcinoma.

11. The method of claim 4, wherein the gastric cancer comprises stomach cancer, gastric lymphoma, gastrointestinal stromal tumor (GIST), or neuroendocrine (carcinoid) tumor.

12. The method of claim 4, wherein the sarcoma comprises soft tissue sarcoma and osteosarcoma.

13. The method of claim 4, wherein the leukemia comprises myeloid leukemia and lymphoid leukemia.

14. The method of claim 1, wherein the ASPH inhibitor has a structure of: 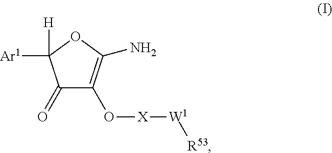
or a pharmaceutically acceptable salt thereof,
wherein
Ar¹ is substituted or unsubstituted C₆-C₂₀ aryl or 5 to 20-membered heteroaryl;
X is —C(O)—, —C(S)—, or —S(O)₂—;
W¹ is a single bond, —O—, —CR⁵⁰R⁵¹—, or —NR⁵²— when X is —C(O)—, or W¹ is a single bond, —CR⁵⁰R⁵¹—, or —NR⁵²— when X is —SO₂—; and
each of R⁵⁰, R⁵¹, R⁵², and R⁵³ independently is selected from the group consisting of hydrogen, substituted or unsubstituted C₁-C₆ alkyl, substituted or unsubstituted C₂-C₆ alkenyl, substituted or unsubstituted C₂-C₆ alkynyl, substituted or unsubstituted C₆-C₂₀ aryl, substituted or unsubstituted C₇-C₂₆ arylalkyl, substituted or unsubstituted 5 to 20-membered heteroaryl, and substituted or unsubstituted 6-26 membered heteroarylalkyl.

15. The method of claim 14, wherein R⁵³ is unsubstituted C₁-C₆ alkyl or phenyl, or C₁-C₆ alkyl or phenyl substituted with one or more substituents selected from halogen, —OH, —CN, —COOCH₃, and amino.

16. The method of claim 14, wherein the ASPH inhibitor has a structure of: 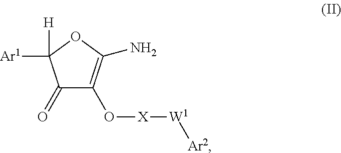
or a pharmaceutically acceptable salt thereof,
wherein
each of Ar¹ and Ar² independently is unsubstituted C₆-C₁₄ aryl, unsubstituted 5 to 14-membered heteroaryl, or C₆-C₁₄ aryl or 5 to 14-membered heteroaryl each substituted with one or more substituents selected from the group consisting of halogen, —CN, —NO₂, —NO, —N₃, —ORₐ, —NRₐR22_{b}, —C(O)Rₐ, —C(O)ORₐ, —C(O)NRₐR_{b}, —NR_{b}C(O)Rₐ, —S(O)_{b}Rₐ, —S(O)_{b}NRₐR_{b}, or —R_{S1}, in which R_{S1} is C₁-C₆ alkyl, C₂-C₆ alkenyl, C₂-C₆ alkynyl, C₃-C₈ cycloalkyl, C₆-C₁₀ aryl, 5- or 6-membered heteroaryl, or 4 to 12-membered heterocycloalkyl, b is 0, 1, or 2, each of Rₐ and R_{b}, independently is H or R_{S2}, and R_{S2} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; and each of R_{S1} and R_{S2}, is optionally substituted with one or more substituents selected from the group consisting of halo, OH, oxo, C(O)OH, C(O)O—C₁-C₆ alkyl, CN, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

17. The method of claim 14, wherein X is C(O) and W¹ is —O—.

18. The method of claim 14, wherein X is —S(O)₂— and W¹ is —CR⁵⁰R⁵¹— or a single bond.

19. The method of claim 14, wherein each of R⁵, R⁵¹,and R⁵² independently is H, unsubstituted C₁-C₆ alkyl, or C₁-C₆ alkyl substituted with one or more substituents selected from halo, OH, CN, and amino.

20. The method of claim 16, wherein each of Ar¹ and Ar² independently is selected from phenyl, 1-naphthyl, 2-naphthyl, 2-furanyl, 2-thiazolyl, 2-pyridyl, 3-pyridyl, 4-pyridyl, 2-quinolinyl, 3-quinolinyl, 4-quinolinyl, 2-chlorophenyl, 3-chlorophenyl, 4-chlorophenyl, 2-fluorophenyl, 3-fluorophenyl, 4-fluorophenyl, 2-trifluoromethylphenyl, 3-trifluoromethylphenyl, 4-trifluoromethylphenyl, 2-cyanophenyl, 3-cyanophenyl, 4-cyanophenyl, 2-carboxymethylphenyl, 3-carboxymethylphenyl, 4-carboxymethylphenyl, 2-methoxyphenyl, 3-methoxyphenyl, 4-methoxyphenyl, 2,3-dichlorophenyl, 2,4-dichlorophenyl, 2,5-dichlorophenyl, 3,4-dichlorophenyl, 3,5-dichlorophenyl, 2,3-difluorophenyl, 2,4-difluorophenyl, 2,5-difluorophenyl, 3,4-difluorophenyl, 3,5-difluorophenyl, 2,3-dimethoxyphenyl, 2,4-dimethoxyphenyl, 2,5-dimethoxyphenyl, 3,4-dimethoxyphenyl, 3,5-dimethoxyphenyl, 2-chloro-6-fluorophenyl, 3-chloro-4-fluorophenyl, 2-chloro-4-fluorophenyl, 4-chloro-3-fluorophenyl, 3-chloro-2-fluorophenyl, 2-chloro-5-fluorophenyl, 4-chloro-2-fluorophenyl, and 5-chloro-2-fluorophenyl.

21. The method of claim 14, wherein the ASPH inhibitor is selected from:

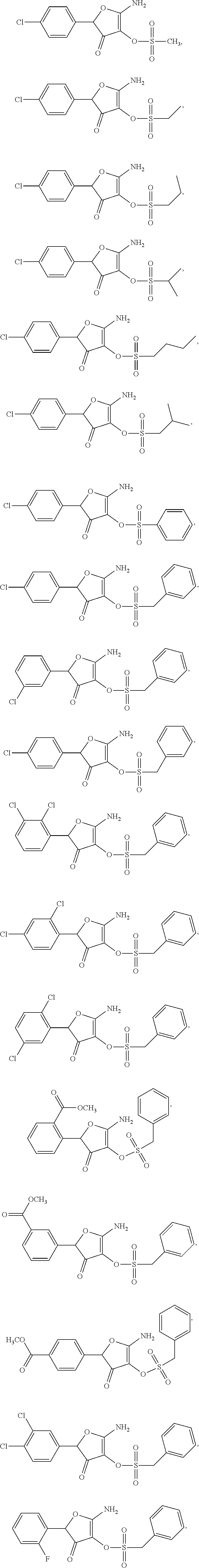

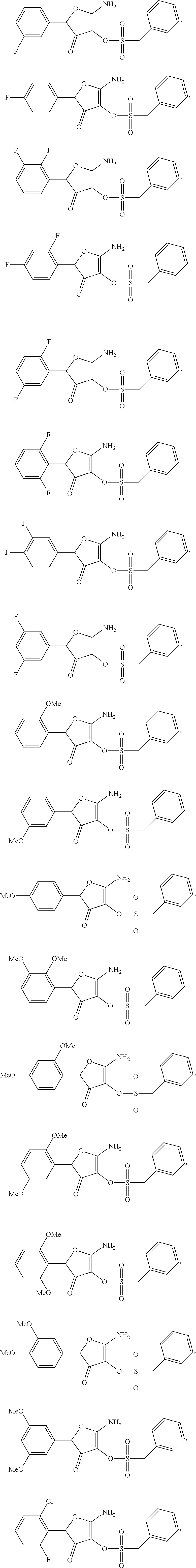

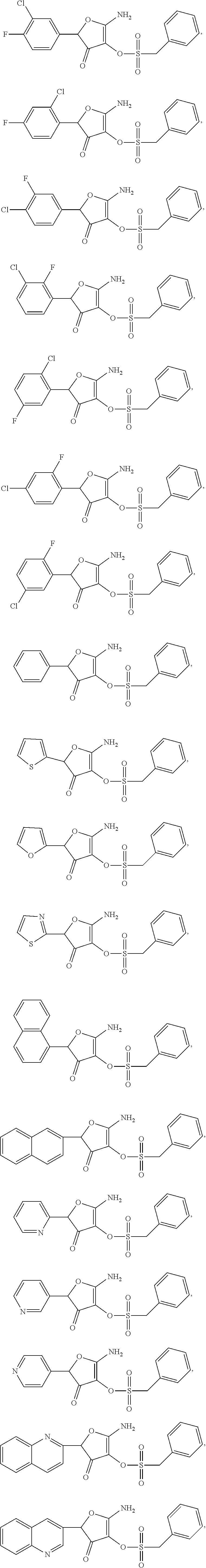

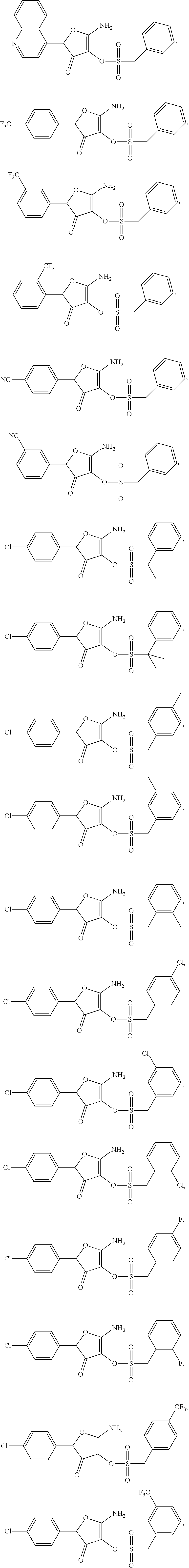

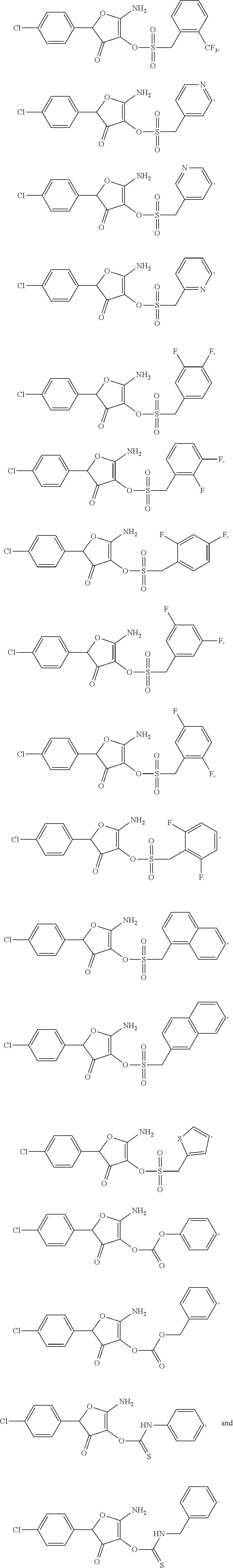

22. The method of claim 1, wherein the ASPH inhibitor is administered intravenously, orally, subcutaneously, intranasally, intraspinally, intrathecally, intramuscularly, intrabronchially, intrarectally, intraocularly, intravaginally, or by surgical implantation.

23. A method of suppressing metastasis in a cancer cell contacting the cancer cell with a therapeutically effective amount of an asparatyl (asparaginyl) beta-hydroxylase (ASPH) inhibitor.

24. The method of claim 23, wherein the ASPH inhibitor has a structure of

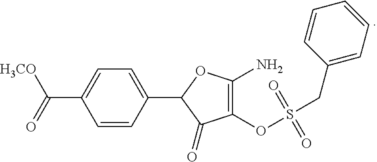 [SMILES: COC(=O)c1ccc(C2OC(N)=C(OS(=O)(=O)Cc3ccccc3)C2=O)cc1]

25. The method of claim 23, wherein the cancel cell expresses greater level of ASPH than a normal cell.

26. The method of claim 25, wherein the cancer cell is from liver cancer, pancreatic cancer, lung cancer, colon cancer, breast cancer, gastric cancer, sarcoma, renal cancer, prostate cancer, brain cancer, or leukemia.

27. The method of claim 26, wherein the liver cancer comprises hepatocellular cancer, or cholangiocarcinoma.

28. The method of claim 26, wherein the lung cancer comprises small cell lung cancer, non-small cell lung cancer, or metastatic lung cancer.

29. The method of claim 26, wherein the brain cancer comprises gliomas, meningioma, astrocytomas, or glioblastoma.

30. The method of claim 26, wherein the breast cancer comprises ductal carcinoma, triple negative breast cancer, inflammatory breast cancer, metastatic breast cancer, medullary carcinoma, tubular carcinoma, or mucinous carcinoma.

31. The method of claim 26, wherein the gastric cancer comprises stomach cancer, gastric lymphoma, gastrointestinal stromal tumor (GIST), or neuroendocrine (carcinoid) tumor.

32. The method of claim 26, wherein the sarcoma comprises soft tissue sarcoma and osteosarcoma.

33. The method of claim 26, wherein the leukemia comprises myeloid leukemia or lymphoid leukemia.

34. The method of claim 23, wherein the ASPH inhibitor has a structure of: 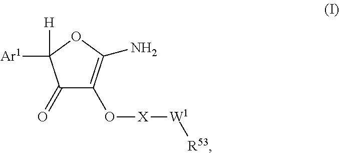
or a pharmaceutically acceptable salt thereof,
wherein
Ar¹ is substituted or unsubstituted C₆-C₂₀ aryl or 5 to 20-membered heteroaryl;
X is —C(O)—, —C(S)—, or —S(O)₂—;
W¹ is a single bond, —O—, —CR⁵⁰R⁵¹—, or —NR⁵²— when X is —C(O)—, or W¹ is a single bond, —CR⁵⁰R⁵¹—, or —NR⁵²— when X is —SO₂—; and
each of R⁵⁰, R⁵¹, R⁵², and R⁵³ independently is selected from the group consisting of hydrogen, substituted or unsubstituted C₁-C₆ alkyl, substituted or unsubstituted C₂-C₆ alkenyl, substituted or unsubstituted C₂-C₆ alkynyl, substituted or unsubstituted C₆-C₂₀ aryl, substituted or unsubstituted C₇-C₂₆ arylalkyl, substituted or unsubstituted 5 to 20-membered heteroaryl, and substituted or unsubstituted 6-26 membered heteroarylalkyl.

35. The method of claim 34, wherein R⁵³ is unsubstituted C₁-C₆ alkyl or phenyl, or C₁-C₆ alkyl or phenyl substituted with one or more substituents selected from halogen, —OH, —CN, —COOCH₃, and amino.

36. The method of claim 34, wherein the ASPH inhibitor has a structure of: 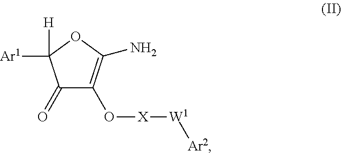
or a pharmaceutically acceptable salt thereof,
wherein
each of Ar¹ and Ar² independently is unsubstituted C₆-C₁₄ aryl, unsubstituted 5 to 14-membered heteroaryl, or C₆-C₁₄ aryl or 5 to 14-membered heteroaryl each substituted with one or more substituents selected from the group consisting of halogen, —CN, —NO₂, —NO, —N₃, —ORₐ, —NRₐR_{b}, —C(O)Rₐ, —C(O)ORₐ, —C(O)NRₐR_{b}, —NR_{b}C(O)Rₐ, —S(O)_{b}Rₐ, —S(O)_{b}NRₐR_{b}, or —R_{S1}, in which R_{S1} is C₁-C₆ alkyl, C₂-C₆ alkenyl, C₂-C₆ alkynyl, C₃-C₈ cycloalkyl, C₆-C₁₀ aryl, 5- or 6-membered heteroaryl, or 4 to 12-membered heterocycloalkyl, b is 0, 1, or 2, each of Rₐ and R_{b}, independently is H or R_{S2}, and R_{S2} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; and each of R_{S1} and R_{S2}, is optionally substituted with one or more substituents selected from the group consisting of halo, OH, oxo, C(O)OH, C(O)O—C₁-C₆ alkyl, CN, C₁-C₆ alkyl, C₁-C₆ alkoxyl, amino, mono-C₁-C₆ alkylamino, di-C₁-C₆ alkylamino, C₃-C₈ cycloalkyl, C₆-C₁o aryl, 4 to 12-membered heterocycloalkyl, and 5- or 6-membered heteroaryl.

37. The method of claim 34, wherein X is C(O) and W¹ is —O—.

38. The method of claim 34, wherein X is —S(O)₂— and W¹ is —CR⁵⁰R⁵¹— or a single bond.

39. The method of claim 34, wherein each of R⁵, R⁵¹,and R⁵² independently is H, unsubstituted C₁-C₆ alkyl, or C₁-C₆ alkyl substituted with one or more substituents selected from halo, OH, CN, and amino.

40. The method of claim 36, wherein each of Ar¹ and Ar² independently is selected from phenyl, 1-naphthyl, 2-naphthyl, 2-furanyl, 2-thiazolyl, 2-pyridyl, 3-pyridyl, 4-pyridyl, 2-quinolinyl, 3-quinolinyl, 4-quinolinyl, 2-chlorophenyl, 3-chlorophenyl, 4-chlorophenyl, 2-fluorophenyl, 3-fluorophenyl, 4-fluorophenyl, 2-trifluoromethylphenyl, 3-trifluoromethylphenyl, 4-trifluoromethylphenyl, 2-cyanophenyl, 3-cyanophenyl, 4-cyanophenyl, 2-carboxymethylphenyl, 3-carboxymethylphenyl, 4-carboxymethylphenyl, 2-methoxyphenyl, 3-methoxyphenyl, 4-methoxyphenyl, 2,3-dichlorophenyl, 2,4-dichlorophenyl, 2,5-dichlorophenyl, 3,4-dichlorophenyl, 3,5-dichlorophenyl, 2,3-difluorophenyl, 2,4-difluorophenyl, 2,5-difluorophenyl, 3,4-difluorophenyl, 3,5-difluorophenyl, 2,3-dimethoxyphenyl, 2,4-dimethoxyphenyl, 2,5-dimethoxyphenyl, 3,4-dimethoxyphenyl, 3,5-dimethoxyphenyl, 2-chloro-6-fluorophenyl, 3-chloro-4-fluorophenyl, 2-chloro-4-fluorophenyl, 4-chloro-3-fluorophenyl, 3-chloro-2-fluorophenyl, 2-chloro-5-fluorophenyl, 4-chloro-2-fluorophenyl, and 5-chloro-2-fluorophenyl.

41. The method of claim 34, wherein the ASPH inhibitor is selected from:

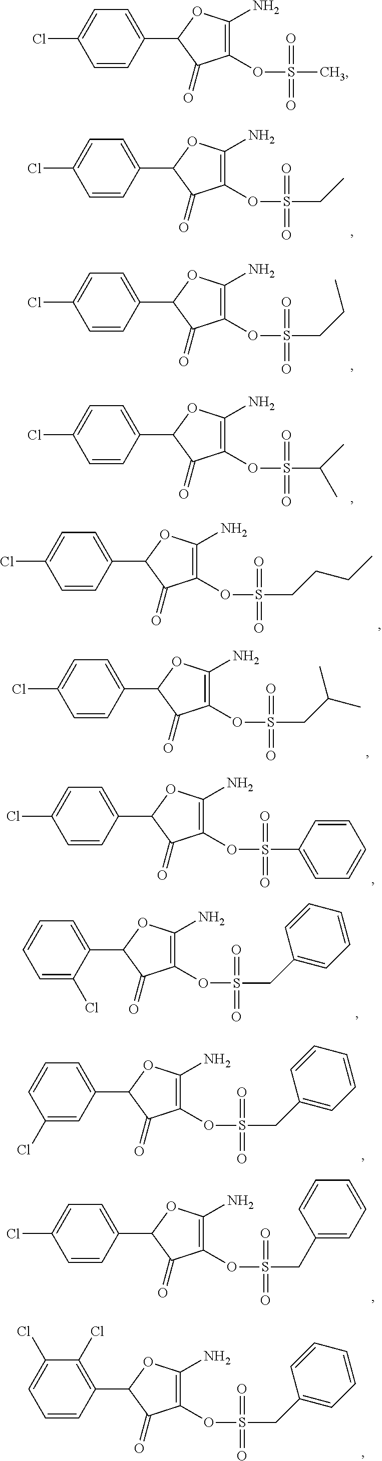

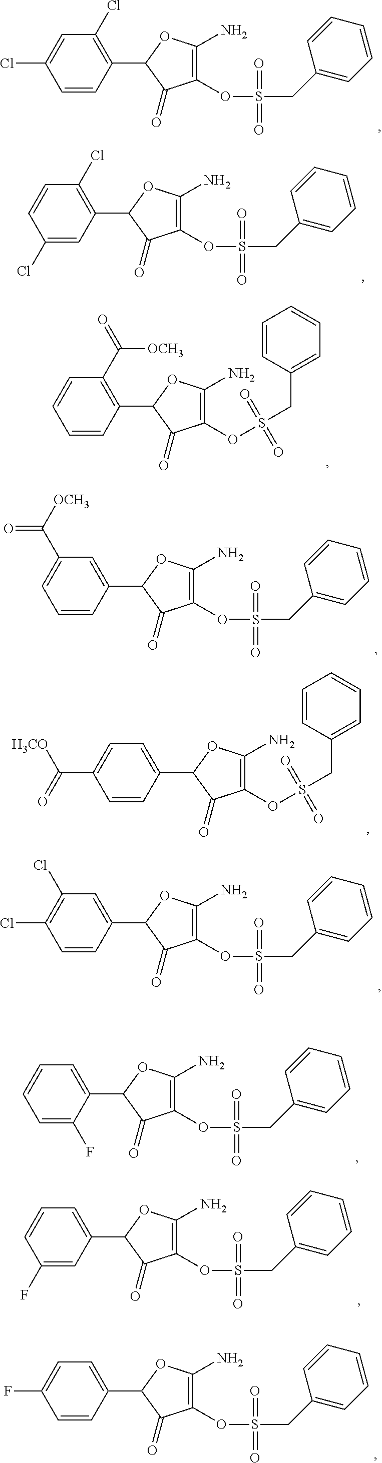

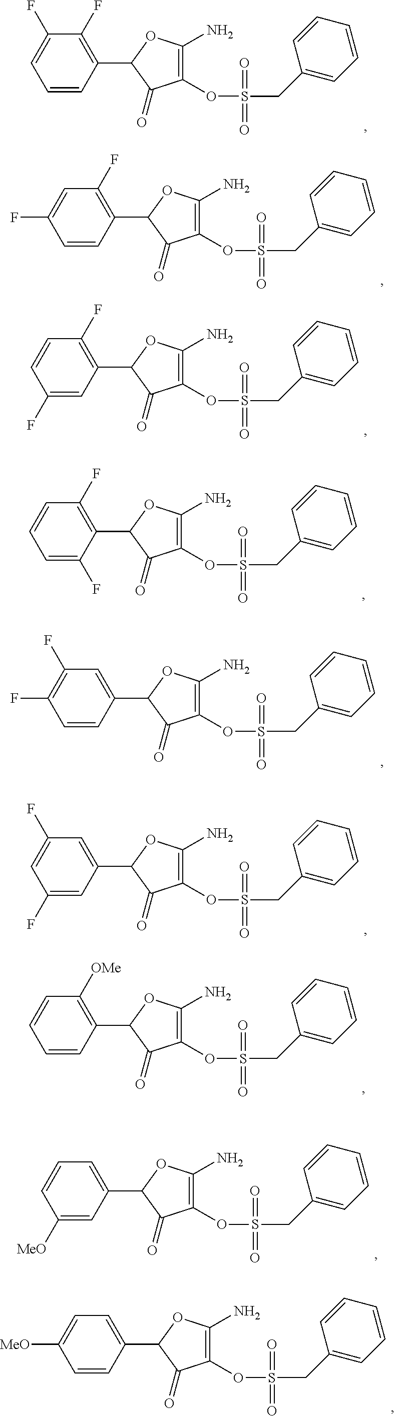

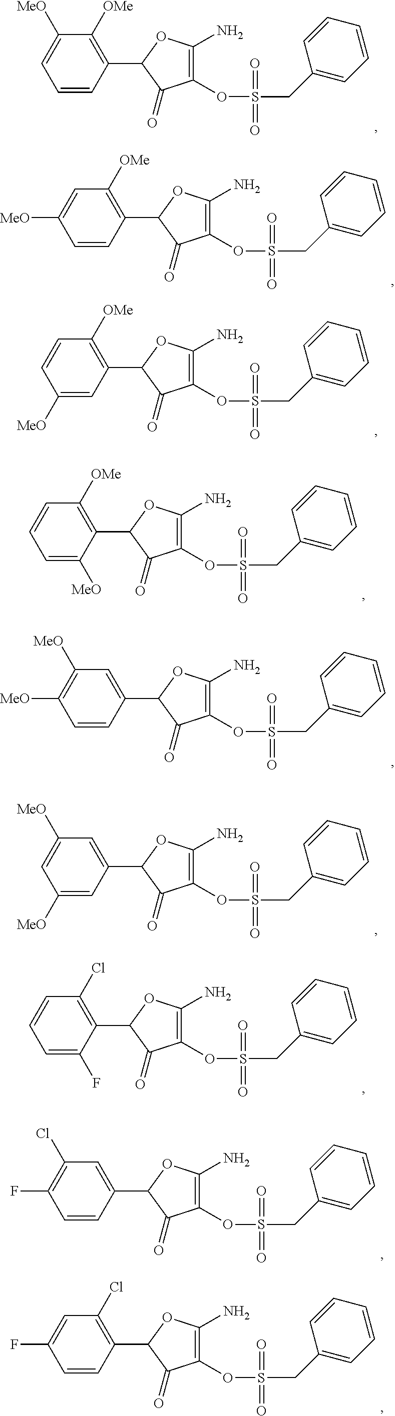

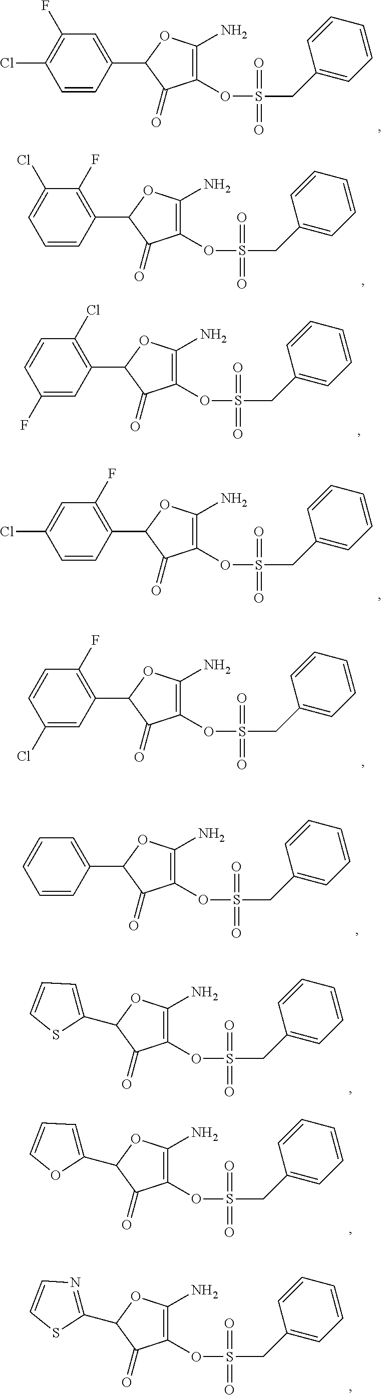

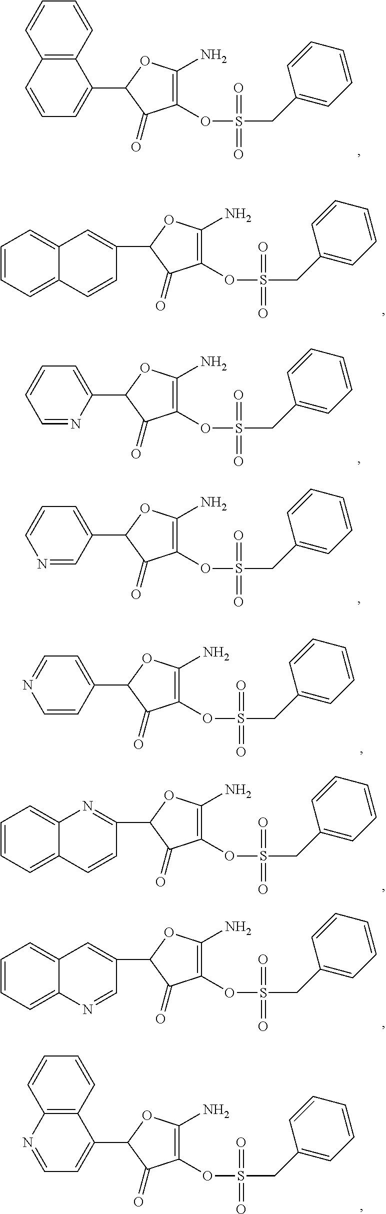

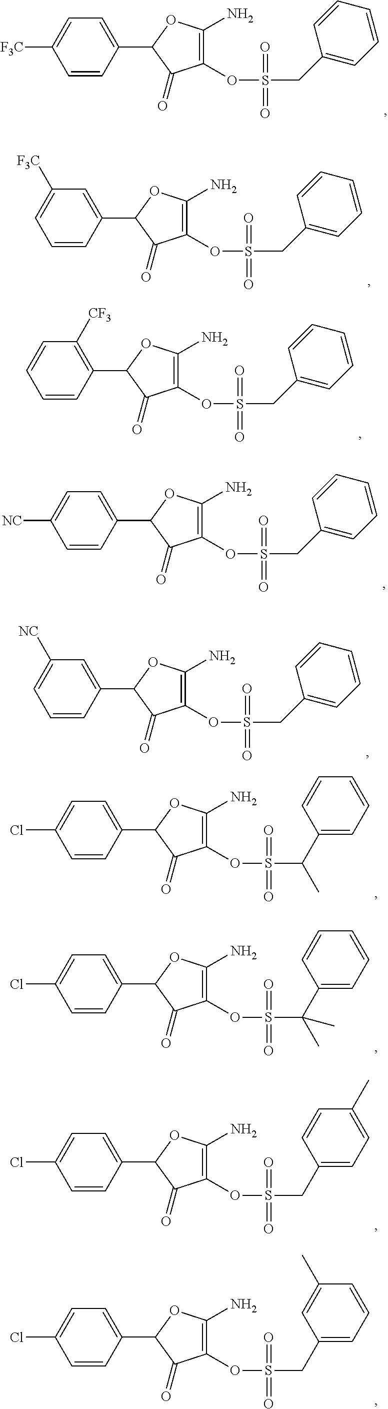

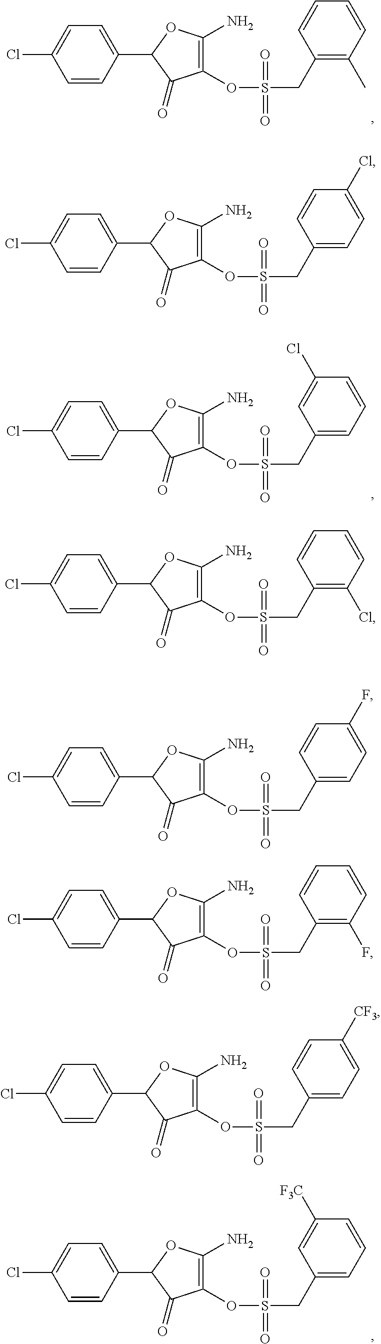

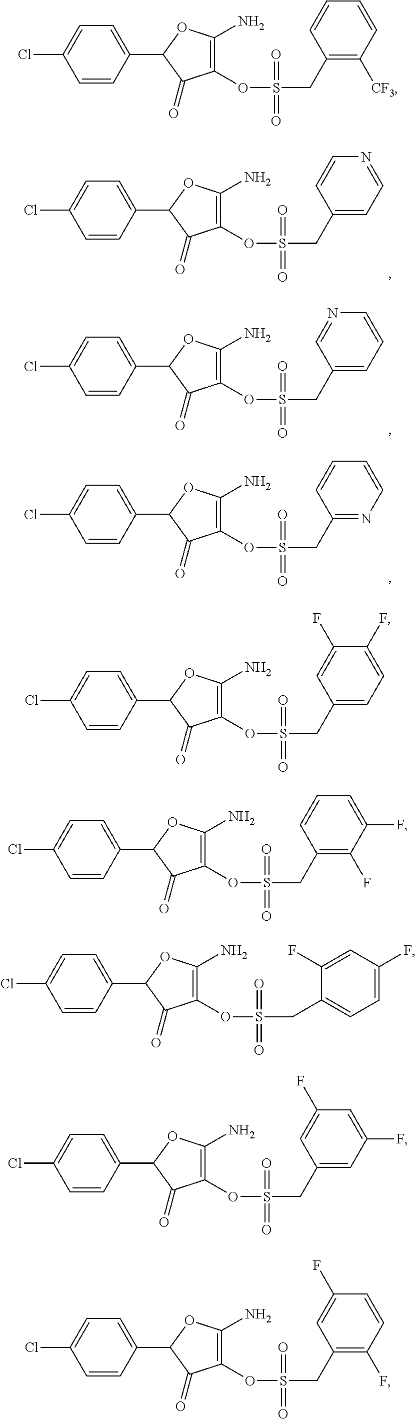

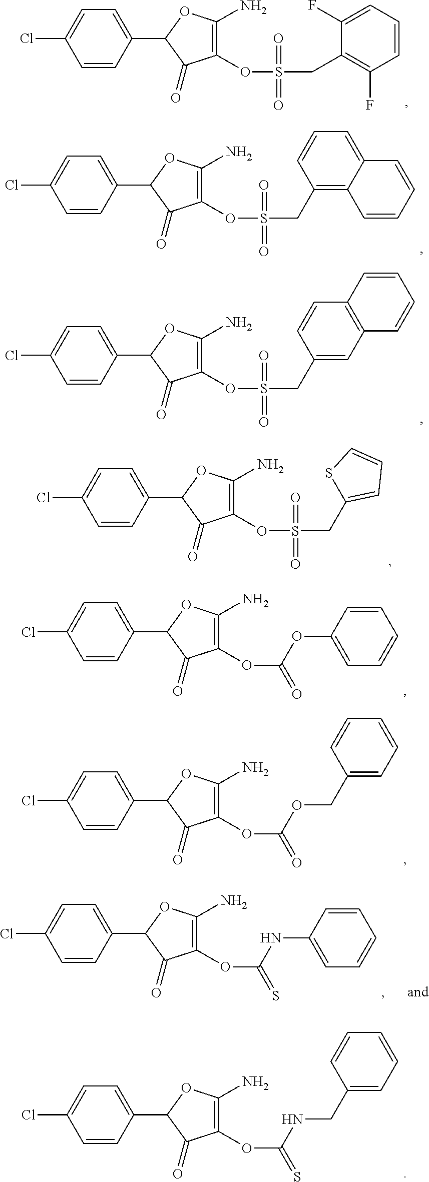

42. The method of claim 23, wherein the ASPH inhibitor is administered intravenously, orally, subcutaneously, intranasally, intraspinally, intrathecally, intramuscularly, intrabronchially, intrarectally, intraocularly, intravaginally, or by surgical implantation.
